SEQUENCE LISTING (17 sequences)

SEQ ID NO: 1 - DNA, 4506 bp, synthetic construct
  source 1..4506: mol_type = other DNA
agaggaaataagagagaaaagaagagtaagaagaaatataagagccaccatggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 4506 nt, synthetic construct
  source 1..4506: mol_type = other RNA
agaggaaataagagagaaaagaagagtaagaagaaatataagagccaccatggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 4203 bp, synthetic construct
  source 1..4203: mol_type = other DNA
atggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatga

SEQ ID NO: 4 - RNA, 4203 nt, synthetic construct
  source 1..4203: mol_type = other RNA
atggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatga

SEQ ID NO: 5 - protein, 1400 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 6 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 16 aa, synthetic construct
KRPAATKKAGQAKKKK

SEQ ID NO: 8 - protein, 7 aa, synthetic construct
PKKKRKV

SEQ ID NO: 9 - DNA, 49 bp, synthetic construct
  source 1..49: mol_type = other DNA
agaggaaataagagagaaaagaagagtaagaagaaatataagagccacc

SEQ ID NO: 10 - RNA, 49 nt, synthetic construct
  source 1..49: mol_type = other RNA
agaggaaataagagagaaaagaagagtaagaagaaatataagagccacc

SEQ ID NO: 11 - DNA, 114 bp, synthetic construct
  source 1..114: mol_type = other DNA
gcggccgcttaattaagctgccttctgcggggcttgccttctggccatgcccttcttctctcccttgcacctgtacctcttggtctttgaataaagcctgagtaggaagtctag

SEQ ID NO: 12 - RNA, 114 nt, synthetic construct
  source 1..114: mol_type = other RNA
gcggccgcttaattaagctgccttctgcggggcttgccttctggccatgcccttcttctctcccttgcacctgtacctcttggtctttgaataaagcctgagtaggaagtctag

SEQ ID NO: 13 - RNA, 140 nt, synthetic construct
  source 1..140: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - RNA, 4504 nt, synthetic construct
  source 1..4504: mol_type = other RNA
aggaaataagagagaaaagaagagtaagaagaaatataagagccaccatggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 47 nt, synthetic construct
  source 1..47: mol_type = other RNA
aggaaataagagagaaaagaagagtaagaagaaatataagagccacc

SEQ ID NO: 16 - DNA, 4444 bp, synthetic construct
  source 1..4444: mol_type = other DNA
aggaaataagagagaaaagaagagtaagaagaaatataagagccaccatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 4444 nt, synthetic construct
  source 1..4444: mol_type = other RNA
aggaaataagagagaaaagaagagtaagaagaaatataagagccaccatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0145976 A1
(43) Pub. Date: May 8, 2025

(54) MESSENGER RNA ENCODING CAS9 FOR USE IN GENOME-EDITING SYSTEMS

(71) Applicant: CRISPR THERAPEUTICS AG, Zug (CH)

(72) Inventor: Christian Cobaugh, Cambridge, MA (US)

(21) Appl. No.: 19/008,854

(22) Filed: Jan. 3, 2025

Related U.S. Application Data
(63) Continuation of application No. 17/319,291, filed on May 13, 2021, now Pat. No. 12,188,060.
(60) Provisional application No. 63/025,755, filed on May 15, 2020.

(51) Int. Cl. C12N 9/22; A61K 9/51; A61K 31/7088; A61K 48/00; C12N 15/11; C12N 15/90

(52) U.S. Cl. CPC C12N 9/22; A61K 9/5123; A61K 31/7088; C12N 15/11; C12N 15/907; A61K 48/00; C12N 2310/20; C12N 2320/32; C12N 2800/80

(57) ABSTRACT

The present disclosure provides optimized mRNAs encoding a site-directed endonuclease for use in a CRISPR/Cas system. Also provided herein are delivery systems for use of the CRISPR/Cas system in methods of in vivo and ex vivo genome editing.

### CROSS REFERENCE TO RELATED APPLICATIONS

The present application is a continuation of U.S. patent application Ser. No. 17/319,291, filed May 13, 2021, allowed as U.S. Pat. No. 12,188,060, which claims the benefit of priority to U.S. Provisional Patent Application No. 63/025,755, filed May 15, 2020, the disclosure of which are incorporated herein by reference in their entireties.

### INCORPORATION BY REFERENCE OF MATERIAL SUBMITTED ELECTRONICALLY

The Sequence Listing, which is a part of the present disclosure, is submitted concurrently with the specification. The name of the text file containing the Sequence Listing is “CRISP_43578_303_SequenceListing.xml”, which was created on Jan. 3, 2025 and is 49,801 bytes in size. The subject matter of the Sequence Listing is incorporated herein in its entirety by reference.

### BACKGROUND

Recent advances in genome sequencing techniques and analysis methods have significantly accelerated the ability to identify and map genetic elements associated with a diverse range of biological functions and diseases. Precise genome targeting technologies are needed to enable reverse engineering of causal genetic variations by allowing selective perturbation of individual genetic elements, as well as to advance synthetic biology, biotechnological, and medical applications. In recent years, targeted genome editing technologies using engineered nucleases have progressed from being niche technologies to advanced methods used by many biological researchers. This adoption has been largely fueled by the emergence of a new class of site-specific endonucleases, including designer zinc fingers, transcription activator-like effectors (TALEs), homing meganucleases, and the development of the clustered, regularly interspaced, short palindromic repeat (CRISPR) technology.

The CRISPR/Cas9 system, which includes an RNA-guided nuclease (Cas9) and one or more guide RNAs (gRNAs), has become a powerful tool for manipulating/editing genomes. Upon delivery of Cas9 polypeptide and gRNA to the nuclease of a cell, the gRNA directs Cas9 to a target gene sequence and the Cas9/gRNA complex generates a site-specific DNA double-strand break (DSB). These DSBs are repaired by endogenous cellular mechanisms, including non-homologous end joining (NHEJ) and homology directed repair (HDR), which can, for example, introduce a mutation in the target gene through formation of insertions or deletions (indels) at the DSB or introduce an exogenous nucleotide sequence by insertion at the DSB.

Application of the CRISPR/Cas9 system depends upon effective delivery, as well as expression/activity, of system components to target cells, which can be challenging for large biomolecules such as Cas9. Various methods of introducing Cas9 to target cells have been explored, but each have drawbacks. For example, plasmid or viral vectors have been used for Cas9 delivery. However, such methods suffer the risk of vector integration into the genome. Recombinant Cas9 polypeptide complexed to gRNA (i.e., ribonucleoprotein or RNP complexes) has also been used for delivery. However, the stability of such complexes in cells or plasma is limited, which can be detrimental for certain genome editing applications. In addition, mRNA expressing Cas9 can induce innate immune responses, reducing Cas9 expression. Thus, there remains a need for compositions and methods that enable efficient delivery and expression/activity of CRISPR/Cas9 system components to target cells and tissues for broad application to methods of genome editing.

### SUMMARY

The present disclosure provides optimized messenger RNAs (mRNAs) encoding a site-directed endonuclease, such as an S. pyogenes Cas9 endonuclease (“SpCas9 mRNA”), which, when combined with one or gRNAs provide effective genome editing of target cells. In some aspects, the disclosure provides an mRNA comprising a 5′ untranslated region (UTR); an open reading frame (ORF) comprising a nucleotide sequence that encodes a site-directed endonuclease, wherein the nucleotide sequence is at least 85% identical to a nucleotide sequence of SEQ ID NO: 4; and a 3′ untranslated region (UTR). In some aspects, the mRNA comprises at least one chemically modified nucleoside. In some aspects, the chemically modified nucleoside is selected from pseudouridine, N1-methylpseudouridine, and 5-methoxyuridine. In some aspects, the chemically modified nucleoside is N1-methylpseudouridine. In some aspects, at least about 80% of the uridines are chemically modified. In some aspects, 100% of the uridines are chemically modified. In some aspects, the uridines are modified and/or replaced with N1-methylpseudouridine.

In some aspects, the disclosure provides an mRNA comprising: a 5′ UTR; an ORF comprising a nucleotide sequence that encodes a site-directed endonuclease, wherein the nucleotide sequence is at least 85% identical to the nucleotide sequence of SEQ ID NO: 4; and a 3′ UTR, wherein 100% of the uridines of the mRNA are modified and/or replaced with N1-methylpseudouridine.

In any of the foregoing or related aspects, the 5′ UTR comprises a nucleotide sequence of SEQ ID NO: 10 or SEQ ID NO: 15. In some aspects, the 3′ UTR comprises a nucleotide sequence of SEQ ID NO: 12.

In any of the foregoing or related aspects, the mRNA further comprises a poly-A tail. In some aspects, the poly-A tail is about 100 to about 1000, about 10 to about 500, about 10 to about 300, about 10 to about 200, about 50 to about 200, about 50 to about 150, about 100 to about 150, or about 120 to about 150 adenosine nucleotides.

In any of the foregoing or related aspects, the mRNA comprises the nucleotide sequence of SEQ ID NO: 2 or SEQ ID NO: 14.

In some aspects, the disclosure provides an mRNA comprising a nucleotide sequence of SEQ ID NO: 2 or SEQ ID NO: 14, wherein 100% of the uridines of the mRNA are modified and/or replaced with N1-methylpseudouridine.

In any of the foregoing or related aspects, the mRNA comprises a 5′ cap. In some aspects, the 5′ cap is a cap-0, a cap-1, or a cap-2 structure.

In some aspects, the disclosure provides a system for editing a target gene in a genomic DNA molecule in a cell, the system comprising (a) an mRNA described herein; and (b) at least one guide RNA (gRNA) directed to the target gene, wherein the mRNA is translated when the mRNA contacts the cell and provides a site-directed endonuclease that combines with the gRNA to induce a double-stranded DNA break (DSB) at a site in the target gene.

In some aspects, the disclosure provides a system for introducing a double-stranded DNA break (DSB) in a target gene in a cell, the system comprising (a) an mRNA described herein; and (b) at least one guide RNA (gRNA) directed to the target gene, wherein the mRNA is translated when the mRNA contacts the cell and provides a site-directed endonuclease that combines with the gRNA to induce a DSB at a site in the target gene.

In some aspects, the disclosure provides a system for correcting a mutation in a target gene in a cell, the system comprising: (a) an mRNA described herein; (b) at least one gRNA directed to the target gene; and (c) a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) or homology-directed DNA repair pathway inserts the donor polynucleotide into the DSB at a location proximal to the mutation, thereby correcting the mutation.

In any of the foregoing or related aspects, the mRNA and the gRNA are individually formulated in a lipid nanoparticle (LNP). In some aspects, the mRNA and the gRNA are co-formulated in an LNP. In some aspects, the donor polynucleotide is individually formulated in a LNP. In some aspects, the donor polynucleotide is encoded by an AAV.

In any of the foregoing or related aspects, the LNP comprises one or more lipid moieties selected from: an amino lipid, an ionizable lipid, a neutral lipid, a PEG-lipid, a helper lipid, a cholesterol or derivative thereof.

In some aspects, the disclosure provides a pharmaceutical composition comprising: an mRNA described herein or a system described herein, and a pharmaceutically acceptable carrier. In some aspects, the pharmaceutical composition further comprises at least one gRNA directed to a target gene. In some aspects, the pharmaceutical composition further comprises a donor polynucleotide.

In some aspects, the disclosure provides a method for editing a target gene in a genomic DNA molecule in a cell, the method comprising: contacting the cell with: (i) an mRNA described herein and at least one gRNA directed to the target gene; (ii) a system described herein; or (iii) a pharmaceutical composition described herein, wherein the mRNA is translated when the mRNA, the system, or the composition contacts the cell and provides a site-directed endonuclease that combines with the gRNA to induce a DSB at a site in the target gene.

In some aspects, the disclosure provides a method for inducing a DSB in a target gene in a cell, the method comprising: contacting the cell with: (i) an mRNA described herein and at least one gRNA directed to the target gene; (ii) a system described herein; or (iii) a pharmaceutical composition described herein, wherein the mRNA is translated when the mRNA, the system, or the composition contacts the cell and provides a site-directed endonuclease that combines with the gRNA to induce a DSB at a site in the target gene.

In some aspects, the disclosure provides a method of treating a patient with a disease by editing a target gene in a genomic DNA molecule in a cell, the method comprising: isolating a cell from the patient, and contacting the cell with: (i) an mRNA described herein and at least one gRNA directed to the target gene; (ii) a system described herein; or (iii) a pharmaceutical composition described herein, wherein the mRNA is translated when the mRNA, system, or composition contacts the cell and provides a site-directed endonuclease that combines with the gRNA to induce a DSB at a site in the target gene.

In some aspects, the disclosure provides a method of treating a patient with a disease by inducing a DSB in a target gene in a cell, the method comprising: isolating a cell from the patient, and contacting the cell with: (i) an mRNA described herein and at least one gRNA directed to the target gene; (ii) a system described herein; or (iii) a pharmaceutical composition described herein, wherein the mRNA is translated when the mRNA, system, or composition contacts the cell and provides a site-directed endonuclease that combines with the gRNA to induce a DSB at a site in the target gene.

In some aspects, the disclosure provides a method of treating a patient with a disease by editing a target gene in a genomic DNA molecule in a cell, the method comprising: administering to the patient an effective amount of (i) an mRNA described herein and at least one gRNA directed to the target gene; (ii) a system described herein; or (iii) a pharmaceutical composition described herein, wherein the mRNA is translated when the mRNA, system, or composition contacts the cell and provides a site-directed endonuclease that combines with the gRNA to induce a DSB at a site in the target gene.

In some aspects, the disclosure provides a method of treating a patient with a disease by inducing a DSB in a target gene in a cell, the method comprising: administering to the patient an effective amount of (i) an mRNA described herein and at least one gRNA directed to the target gene; (ii) a system described herein; or (iii) a pharmaceutical composition described herein, wherein the mRNA is translated when the mRNA, system, or composition contacts the cell and provides a site-directed endonuclease that combines with the gRNA to induce a DSB at a site in the target gene.

In some aspects, the disclosure provides a method for correcting a mutation in a target gene in a cell, the method comprising: contacting the cell with an mRNA described herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) or homology-directed DNA repair pathway inserts the donor polynucleotide into the DSB at a location proximal to the mutation, thereby correcting the mutation.

In some aspects, the disclosure provides a method of treating a patient with a disease by correcting a mutation in a target gene in a cell, the method comprising: isolating a cell from the patient; and contacting the cell with an mRNA described herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) or homology-directed DNA repair pathway inserts the donor polynucleotide into the DSB at a location proximal to the mutation, thereby correcting the mutation.

In some aspects, the disclosure provides a method of treating a patient with a disease by correcting a mutation in a target gene in a cell, the method comprising: administering to the patient an effective amount of an mRNA described herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) or homology-directed DNA repair pathway inserts the donor polynucleotide into the DSB at a location proximal to the mutation, thereby correcting the mutation.

In some aspects, the disclosure provides a kit for inducing a DSB in a target gene in a cell, the kit comprising: a container comprising an mRNA described herein, a system described herein, or a pharmaceutical composition described herein, and a package insert comprising instructions for use.

### BRIEF DESCRIPTION OF THE DRAWINGS

FIG. 1 provides a graph showing frequency of INDELs in liver tissue of mice administered lipid nanoparticles (LNPs) containing gRNA targeting the mouse transferrin gene locus (mTF_T2 sgRNA) and sequence-optimized mRNA encoding SpCas9 (RNA-009 mRNA). RNA-009 mRNA contained either unmodified uridine (RNA-009u), N1-methylpseudouridine (RNA-009n), or 5-methoxyuridine (RNA-009m). Comparison was made to control mice administered PBS only.

FIGS. 2A-2B provide graphs quantifying levels of IL-6 (FIG. 2A) and MCP-1 (FIG. 2B) in serum isolated from the mice of FIG. 1.

FIG. 3 provides a graph showing frequency of INDELs in liver tissue of mice administered LNPs containing mTF_T2 sgRNA and SpCas9 mRNA that was either RNA-009n, RNA-012n, or RNA-013n. RNA-012n and RNA-013n encoded a sequence optimized, uridine-depleted mRNA sequence and were modified throughout with N1-methylpseudouridine. Comparison was made to control mice administered PBS only.

FIGS. 4A-4B provide graphs quantifying levels of IL-6 (FIG. 4A) and MCP-1 (FIG. 4B) in serum isolated from the mice of FIG. 3.

FIGS. 5A-5C provides a graph showing frequency of INDELs in liver tissue of mice administered with various dose levels of LNPs containing RNA-009n mRNA and sgRNA targeting the albumin (FIG. 5A), C3 (FIG. 5B), or transferrin (FIG. 5C) loci.

FIG. 6 provides a graph quantifying SpCas9 protein in liver tissue of mice administered LNP containing RNA-009n mRNA. Quantification is shown as pg of SpCas9 per mg of tissue (pg/mg) for 2 and 6 hours following LNP administration.

FIG. 7 provides a graph showing frequency of INDELs in liver tissue of non-human primates (NHPs) administered LNPs containing sgRNA targeting NHP albumin gene target (hT5) and SpCas9 mRNA that was RNA-012 with unmodified uracil (RNA-012u), RNA-012n, or RNA-009n.

### DETAILED DESCRIPTION

### Overview

The present disclosure provides optimized mRNAs encoding an S. pyogenes Cas9 endonuclease (“SpCas9 mRNA”), and which optionally include chemically modified nucleotides, that provide effective genome editing of a target cell population when administered with one or more gRNAs. As described herein, approaches were discovered that resulted in an optimized SpCas9 mRNA with improved translation and/or SpCas9 polypeptide activity. Without being bound by theory, it is believed that such optimized mRNAs increase translation in target cells and tissues and reduces activation of RNA-responsive innate immune response pathways that can trigger an adaptive immune response against the CRISPR/Cas9 system. In some embodiments, the mRNA is codon optimized for effective translation in host cells (e.g., human cells). In some embodiments, the SpCas9 mRNA is chemically modified, for example, to minimize uridine-rich sequences that trigger innate immune response pathways. As described herein, a combination of approaches provided an optimized SpCas9 mRNA, which optionally include chemically modified nucleotides, that was highly effective for use in methods of genome editing when administered in vivo.

In some aspects, the disclosure provides CRISPR/Cas9 systems comprising the optimized SpCas9 mRNA described herein for use in editing a target gene in a cell, e.g., following in vivo or in vitro administration. In some aspects, the CRISPR/Cas9 system comprises an optimized SpCas9 mRNA described herein and one or more gRNAs for introducing a DSB in a target gene in a cell, for example, to introduce a mutation or correct a mutation in a target gene. In some aspects, the CRISPR/Cas9 system further comprises a donor polynucleotide, for example, to introduce a sequence-specific gene-edit by a NHEJ or HDR repair pathway.

In further aspects, the disclosure also provides methods of delivery of CRISPR/Cas9 system components described herein. For example, in some embodiments, the disclosure provides lipid nanoparticle (LNP) formulations for separate or co-formulation of SpCas9 mRNA and one or more gRNAs. Indeed, it was discovered that the LNP formulations described herein comprising an optimized SpCas9 mRNA of the disclosure were highly effective for introducing gene edits when evaluated with gRNAs targeting different gene loci and following in vivo administration in both mouse and non-human primate animal models. Moreover, the level of editing efficiency could be readily controlled by titrating the dose of the optimized SpCas9 mRNA administered (i.e., dose responsive efficacy).

Thus, provided herein, are compositions and methods for delivery of CRISPR/Cas9 system components (e.g., SpCas9 mRNA and one or more gRNAs) for use in effective genome editing of target tissues and cells while reducing undesirable immune cell activation.

### Messenger RNAs Encoding a Site-Directed Endonuclease

In some aspects, the disclosure provides an mRNA encoding a site-directed endonuclease, such as a SpCas9 polypeptide, for use in methods of genome editing using a CRISPR/Cas system. In some embodiments, the mRNA comprises a 5′ UTR, an open reading frame (ORF) comprising a nucleotide sequence encoding a site-directed endonuclease, such as a SpCas9 polypeptide, and a 3′ UTR.

In some embodiments, an mRNA of the disclosure comprises at least one modification. In some embodiments, the at least one modification provides (i) improved mRNA stability, for example, in serum or in cells, (ii) improved mRNA translation efficiency, and/or (iii) reduced activation of innate immune signaling pathways compared to an equivalent unmodified mRNA. In some embodiments, the at least one modification improves the level and/or duration of expression of the encoded site-directed endonuclease, such as SpCas9 polypeptide, in a target tissue or cell following systemic administration of the mRNA (e.g., as compared to an equivalent unmodified mRNA). In some embodiments, the at least one modification reduces activation of innate immune cell responses following systemic administration of the mRNA (e.g., as compared to an equivalent unmodified mRNA).

In some embodiments, the at least one modification is selected from: (i) sequence optimization of the mRNA, (ii) chemical modification of at least one nucleotide of the mRNA, or (iii) a combination of (i) and (ii).

### I. Sequence Optimization

In some embodiments, an mRNA of the disclosure comprises a sequence-optimized nucleotide sequence. In some embodiments, the mRNA comprises a nucleotide sequence that is sequence optimized for expression in a target cell. In some embodiments, the target cell is a mammalian cell. In some embodiments, the target cell is a human cell, a murine cell, or a non-human primate (NHP) cell.

A sequence-optimized nucleotide sequence, e.g., a codon-optimized mRNA sequence encoding a site-directed endonuclease, such as a SpCas9 polypeptide, typically is a sequence comprising at least one synonymous nucleobase substitution with respect to a reference sequence (e.g., a non-optimized mRNA sequence encoding a site-directed endonuclease, such as a SpCas9 polypeptide). A sequence-optimized nucleotide sequence can be partially or completely different in sequence from the reference sequence. For example, a reference sequence encoding polyserine uniformly encoded by TCT codons can be sequence-optimized by having 100% of its nucleobases substituted (for each codon, T in position 1 replaced by A, C in position 2 replaced by G, and T in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Some sequence optimization (also sometimes referred to as codon optimization) methods are known in the art and can be useful to achieve one or more desired results. These results can include, e.g., matching codon frequencies in certain tissue targets and/or host organisms to ensure proper folding; uridine depletion; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide.

In some embodiments, an mRNA of the disclosure comprises a nucleotide sequence that is sequence-optimized relative to a reference sequence using a method of sequence optimization. Methods of sequence optimization are known in the art, and include known sequence optimization tools, algorithms and services. Non-limiting examples include services from GeneArt (Life Technologies), DNA2.0 (Menlo Park CA), Geneious®, GeneGPS® (Atum, Newark, CA), and/or proprietary methods. In some embodiments, an mRNA of the disclosure comprises a nucleotide sequence that is sequence-optimized relative to a reference sequence using a method of sequence optimization (e.g., GeneGPS®, e.g., Geneious®). In some embodiments, the method of sequence optimization comprises any one codon optimization algorithm described in U.S. Pat. Nos. 7,561,972; 7,561,973; 8,126,653; and 8,401,798, each of which is incorporated herein by reference. In some embodiments, the nucleotide sequence is (i) sequence-optimized based on codon usage bias in a host cell (e.g., mammalian cell, e.g., human cell, murine cell, non-human primate cell) relative to a reference sequence, (ii) uridine-depleted relative to a reference sequence, or (iii) a combination of (i) and (ii), using a method of sequence optimization (e.g., GeneGPS®, e.g., Geneious®).

In some embodiments, the reference sequence comprises the nucleotide sequence of SEQ ID NO: 17. In some embodiments, the sequence-optimized nucleotide sequence comprises one or more nucleobase substitutions relative to the reference sequence. In some embodiments, the sequence-optimized nucleotide sequence is less than about 95%, about 94%, about 93%, about 92%, about 91%, about 90%, about 89%, about 88%, about 87%, about 86%, about 85%, about 84%, about 83%, about 82%, about 81%, or about 80% identical to the reference sequence. In some embodiments, the sequence-optimized nucleotide sequence is at least about 80%, at least about 81%, at least about 82%, at least about 83%, at least about 84%, at least about 85%, at least about 86%, at least about 87%, at least about 88%, at least about 89, at least about 90%, at least about 91%, at least about 92%, at least about 93%, at least about 94%, at least about 95%, at least about 96%, at least about 97%, at least about 98%, or at least about 99% identical to the reference sequence. In some embodiments, the polypeptide encoded by the sequence-optimized nucleotide sequence is 100% identical to the polypeptide encoded by the reference sequence. In some embodiments, the polypeptide encoded by the sequence-optimized nucleotide sequence is set forth in SEQ ID NO: 5.

In some embodiments, the sequence-optimized nucleotide sequence is uridine depleted, e.g., compared to the reference sequence, e.g., compared to the nucleotide sequence of SEQ ID NO: 17. In some embodiments, the uracil content of the sequence-optimized nucleotide sequence is decreased (e.g., by about 1.1-fold, 1.2-fold, 1.3-fold, 1.4-fold, or about 1.5-fold) compared to the reference sequence, e.g., the nucleotide sequence of SEQ ID NO: 17.

In some embodiments, the sequence-optimized nucleotide sequence is not uridine depleted, e.g., compared to the reference sequence, e.g., compared to the nucleotide sequence of SEQ ID NO: 17. In some embodiments, the uracil content of the sequence-optimized nucleotide sequence is substantially equivalent (e.g., about 95% to about 105% similar) or increased (e.g., by about 1.1-fold, about 1.2-fold, about 1.3-fold, about 1.4-fold, or about 1.5-fold) compared to the reference sequence, e.g., the nucleotide sequence of SEQ ID NO: 17.

In some embodiments, the disclosure provides an mRNA comprising a sequence-optimized nucleotide sequence, wherein the mRNA has one or more improved properties (e.g., compared to an mRNA comprising the reference sequence, e.g., compared to an mRNA comprising the nucleotide sequence of SEQ ID NO: 17). In some embodiments, the one or more improved properties relates to expression efficacy after administration in vivo. In some embodiments, the one or more improved properties include, but are not limited to, increased cutting efficiency and/or activity, improving mRNA stability, increasing translation efficacy in the target tissue or target cell, reducing the number of truncated proteins expressed, improving folding or prevent misfolding of the expressed proteins, reducing toxicity of the expressed products, reducing cell death caused by the expressed products, increasing and/or decreasing protein aggregation, or a combination thereof.

In some embodiments, the sequence-optimized nucleotide sequence is codon optimized for expression in human subjects, having structural and/or chemical features that avoid or reduce one or more of the problems known in the art, for example, features that are useful for optimizing formulation and delivery of mRNA-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

II. Modified mRNAs

In some embodiments, the disclosure provides mRNAs with chemistries suitable for delivery, tolerability, and stability within cells, e.g., following in vivo or in vitro administration. Accordingly, in some embodiments, mRNAs described herein are modified, e.g., comprise a modified sugar moiety, a modified internucleoside linkage, a modified nucleoside, a modified nucleotide and/or combinations thereof. In some embodiments, the modified mRNAs exhibit one or more of the following properties: are not immune stimulatory; are nuclease resistant; have improved cell uptake; have increased half-life; have increased translation efficiency; and/or are not toxic to cells or mammals, e.g., following contact with cells in vivo or ex vivo or in vitro.

Additionally, certain nucleotide and nucleoside modifications have been shown to reduce immune stimulation, e.g., stimulation of innate immune pathways, by exogenous mRNA (see, e.g., Kariko, K, et al (2005) IMMUNITY 23:165; Anderson, et al (2011) NUCLEIC ACIDS RES 39:9329; Warren et al (2010) CELL STEM CELL 7:618).

Accordingly, the disclosure provides mRNA comprising chemical modification of one or more nucleosides/nucleotides. In some embodiments, one or more uridines of the mRNA are chemically-modified or replaced with a chemically-modified nucleoside. In some embodiments, the chemically-modified nucleoside selected from: pseudouridine, N1-methylpseudouridine, and 5-methoxyuridine. In some embodiments, the chemically-modified nucleoside is any one described in WO/2017/181107, WO/2018/144775, or WO/2020/056304, each of which is incorporated by reference herein.

In some embodiments, about 100% of the uridines of the mRNA are chemically-modified. In some embodiments, about 95% of the uridines of the mRNA are chemically-modified. In some embodiments, about 90% of the uridines of the mRNA are chemically-modified. In some embodiments, about 85% of the uridines of the mRNA are chemically-modified. In some embodiments, about 80% of the uridines of the mRNA are chemically-modified.

In some embodiments, about 100% of the uridines of the mRNA are chemically-modified and/or replaced with N1-methylpseudouridine. In some embodiments, about 95% of the uridines of the mRNA are chemically-modified and/or replaced with N1-methylpseudouridine. In some embodiments, about 90% of the uridines of the mRNA are chemically-modified and/or replaced with N1-methylpseudouridine. In some embodiments, about 85% of the uridines of the mRNA are chemically-modified and/or replaced with N1-methylpseudouridine. In some embodiments, about 80% of the uridines of the mRNA are chemically-modified and/or replaced with N1-methylpseudouridine.

In some embodiments, the modified nucleobase is N1-methylpseudouridine, and the mRNA of the disclosure is fully modified with N1-methylpseudouridine. In some embodiments, N1-methylpseudouridine represents from 75-100% of the uracils in the mRNA. In some embodiments, N1-methylpseudouridine represents 100% of the uracils in the mRNA.

In some embodiments, an mRNA of the disclosure is modified in the coding region (e.g., an open reading frame encoding a site-directed endonuclease, such as a SpCas9 polypeptide). In some embodiments, the mRNA is modified in regions besides a coding region. For example, in some embodiments, a 5′ UTR and/or a 3′ UTR are provided, wherein either or both may independently contain one or more different nucleoside modifications. In such embodiments, nucleoside modifications may also be present in the coding region.

Additional modifications of the mRNA encompassed by the present disclosure are further described below.

### III. Messenger RNA Components

In some embodiments, the disclosure provides an mRNA comprising an open-reading frame (ORF), wherein the ORF comprises a nucleotide sequence that encodes a site-directed endonuclease, such as a Cas nuclease, wherein the Cas nuclease is a SpCas9 polypeptide. In some embodiments, the Cas nuclease comprises at least one domain that interacts with a guide RNA (gRNA). Additionally, the Cas nuclease is directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, an mRNA of the disclosure comprises a 5′ untranslated region (5′ UTR), a 3′ untranslated region (3′ UTR), and an ORF comprising a nucleotide sequence encoding a site-directed endonuclease, such as a SpCas9 polypeptide. In some embodiments, the mRNA further comprises a 5′ cap structure, a Kozak or Kozak-like sequence (also known as a Kozak consensus sequence), a polyA sequence (also known as a polyadenylation signal), a nucleotide sequence encoding a nuclear localization signal (NLS), a nucleotide sequence encoding a linker peptide, a nucleotide sequence encoding a tag peptide, or any combination thereof. In some embodiments, the consensus Kozak consensus sequence facilitates the initial binding of mRNA to ribosomes, thereby enhances its translation into a polypeptide product.

In some embodiments, an mRNA of the disclosure comprises any suitable number of base pairs, e.g., thousands (e.g., 4000, 5000, 6000, 7000, 8000, 9000, or 10,000) of base pairs. In some embodiments, the mRNA is about 4.2 kb, about 4.3 kb, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, about 5.5 kb, or more in length.

### A. 5′ and 3′ Untranslated Regions (UTRs)

In some embodiments, the 5′ UTR or 3′ UTR is derived from a human gene sequence. Non-limiting exemplary 5′ UTR and 3′ UTR include those derived from genes encoding a- and β-globin, albumin, HSD17B4, and eukaryotic elongation factor 1a. In addition, viral-derived 5′ UTR and 3′ UTRs can also be used and include orthopoxvirus and cytomegalovirus UTR sequences. In some embodiments, the 5′ UTR comprises the nucleotide sequence of SEQ ID NO: 10. In some embodiments, the 5′ UTR comprises the nucleotide sequence of SEQ ID NO: 15. In some embodiments, the 3′ UTR comprises the nucleotide sequence of SEQ ID NO: 12.

### B. 5′ cap

In some embodiments, an mRNA of the disclosure comprises a 5′ cap structure. A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G (5′) ppp (5′) G, commonly written as m⁷GpppG. This cap is a cap-0 where nucleotide N does not contain 2′OMe, or cap-1 where nucleotide N contains 2′OMe, or cap-2 where nucleotides N and N+1 contain 2′OMe. This cap may also be of the structure m2 7′3 “G (5′) N as incorporated by the anti-reverse-cap analog (ARCA), and may also include similar cap-0, cap-1, and cap-2, etc., structures.

In some embodiments, the 5′cap is a CleanCap® (TriLink Biotechnologies) capping structure. Non-limiting examples of CleanCap® capping structures include CleanCap® Reagent GG (m7G (5′) ppp (5′) (2′OMeG) pG, CleanCap® Reagent AU (m7G (5′) ppp (5′) (2′OMeA) pU, and CleanCap® Reagent AG (m7 (3′OMeG) (5′) ppp (5′) (2′OMeA) pG,

In some embodiments, the 5′ cap may regulate nuclear export; prevent degradation by exonucleases; promote translation; and promote 5′ proximal intron excision. Stabilizing elements for caps include phosphorothioate linkages, boranophosphate modifications, and methylene bridges. In addition, caps may also contain a non-nucleic acid entity that acts as the binding element for eukaryotic translation initiation factor 4E, eIF4E.

### C. Nuclear Localization Signal

In some embodiments, an mRNA of the disclosure further comprises a nucleotide sequence encoding a nuclear localization signal (NLS). In some embodiments, the nuclease is fused with more than one NLS. In some embodiments, one or more NLS is operably-linked to the N-terminus, C-terminus, or both, of the site-directed endonuclease, optionally via a peptide linker. In some embodiments, the NLS comprises a nucleoplasmin NLS and/or a SV40 NLS. In some embodiments, the nucleoplasmin NLS comprises the amino acid sequence of SEQ ID NO: 7. In some embodiments, the SV40 NLS comprises the amino acid sequence of SEQ ID NO: 8. In some embodiments, the mRNA comprises a nucleotide sequence encoding a nucleoplasmin NLS and a nucleotide sequence encoding an SV40 NLS.

### D. Poly-A Tail

In some embodiments, an mRNA of the disclosure comprises a poly(A) tail (i.e., polyA sequence, i.e., polyadenylation signal). In some embodiments, the polyA sequence comprises entirely or mostly of adenine nucleotides or analogs or derivatives thereof. In some embodiments, the poly A sequence is a tail located adjacent (e.g., towards the 3′ end) of a 3′ UTR of an mRNA. In some embodiments, the polyA sequence promotes or increases the nuclear export, translation, and/or stability of the mRNA.

In some embodiments, the poly(A) tail is about 40 to about 300 nucleotides in length. In some embodiments, the tail is about 40 to about 100 nucleotides in length. In some embodiments, the tail is about 100 to about 300 nucleotides in length. In some embodiments, the tail is about 100 to about 200 nucleotides in length. In some embodiments, the tail is about 50 to about 200 nucleotides in length. In some embodiments, the tail is about 50 to about 250 nucleotides in length. In some embodiments, the tail is about 100, 110, 120, 130, 140, 150, 160, 170, 180, 190, or 200 nucleotides in length. In some embodiments, the poly(A) tail comprises modifications to prevent exonuclease degradation, including phosphorotioate linkages and modifications to the nucleobase.

In some embodiments, the poly(A) tail comprises a 3′ “cap” comprising modified or non-natural nucleobases or other synthetic moieties.

IV. Exemplary mRNAs

In some embodiments, the disclosure provides an mRNA comprising (i) a 5′ untranslated region (UTR); (ii) an open reading frame (ORF) comprising a nucleotide sequence that encodes a site-directed endonuclease; and (iii) a 3′ untranslated region (UTR). In some embodiments, the site-directed endonuclease is a Cas nuclease. In some embodiments, the Cas nuclease is a Cas9 polypeptide. In some embodiments, the Cas 9 polypeptide is a Streptococcus pyogenes-derived Cas9 (SpCas9) polypeptide. In some embodiments, the ORF further comprises one or more nucleotide sequences encoding a nuclear localization signal, such as one described herein. In some embodiments, the ORF comprises a nucleotide sequence encoding a site-directed endonuclease, such as a SpCas9 polypeptide and at least one NLS that is a nucleoplasmin and/or SV40 NLS. In some embodiments, the ORF comprises a nucleotide sequence encoding an N-terminal and/or C-terminal NLS operably-linked to a site-directed endonuclease, such as a SpCas9 polypeptide. In some embodiments the ORF comprises a nucleotide sequence encoding an N-terminal SV40 NLS operably-linked to a site-directed endonuclease, such as a SpCas9 polypeptide, and a C-terminal nucleoplasmin NLS operably-linked to the site-directed endonuclease, such as the SpCas9 polypeptide. In some embodiments, the nucleoplasmin NLS comprises the amino acid sequence of SEQ ID NO: 7. In some embodiments, the SV40 NLS comprises the amino acid sequence of SEQ ID NO: 8. In some embodiments, the site-directed endonuclease comprises the amino acid sequence of SEQ ID NO: 6.

In some embodiments, the disclosure provides an mRNA comprising (i) a 5′ UTR; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR.

In some embodiments, the disclosure provides an mRNA comprising (i) a 5′ UTR; (ii) an open reading frame (ORF) comprising a nucleotide sequence that encodes a site-directed endonuclease, such as a SpCas9 polypeptide, wherein the nucleotide sequence is set forth by SEQ ID NO: 4; and (iii) a 3′ UTR.

In some embodiments, the 5′ UTR of any of the foregoing mRNA is a 5′ UTR described herein. In some embodiments, the 3′ UTR of any of the foregoing mRNA is a 3′ UTR described herein.

In some embodiments, the disclosure provides an mRNA comprising (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12.

In some embodiments, the disclosure provides an mRNA comprising (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12.

In some embodiments, the disclosure provides an mRNA comprising (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12.

In some embodiments, the disclosure provides an mRNA comprising (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12.

In some embodiments, any of the foregoing mRNA further comprises a poly-A tail, such as one described herein. In some embodiments, the poly-A tail comprises the nucleotide sequence of SEQ ID NO: 13.

In some embodiments, the disclosure provides an mRNA comprising a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 2. In some embodiments, the disclosure provides an mRNA comprising a nucleotide sequence that is 100% identical to the nucleotide sequence of SEQ ID NO: 2.

In some embodiments, the disclosure provides an mRNA comprising a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of ID NO: 14. In some embodiments, the disclosure provides an mRNA comprising a nucleotide sequence that is 100% identical to the nucleotide sequence of SEQ ID NO: 14.

In some embodiments, any of the foregoing mRNA comprise at least one chemically modified nucleoside. In some embodiments, the chemically modified nucleoside is selected from pseudouridine, N1-methylpseudouridine, and 5-methoxyuridine. In some embodiments, the chemically modified nucleoside is N1-methylpseudouridine. In some embodiments, at least about 80% or more (e.g., about 85%, 90%, 91%, 92%, 93%, 94%, 95%, 96%, 97%, 98%, 99%) of uridines in the mRNA are modified or replaced with N1-methylpseudouridine. In some embodiments, 100% of the uridines in the mRNA are modified or replaced with N1-methylpseudouridine.

In some embodiments, the disclosure provides an mRNA comprising (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N1-methylpseudouridine.

In some embodiments, the disclosure provides an mRNA comprising (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N1-methylpseudouridine.

In some embodiments, the disclosure provides an mRNA comprising (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N1-methylpseudouridine.

In some embodiments, the disclosure provides an mRNA comprising (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N1-methylpseudouridine.

In some embodiments, the disclosure provides an mRNA comprising a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 2, wherein 100% of the uridines of the mRNA are modified or replaced with N1-methylpseudouridine.

In some embodiments, the disclosure provides an mRNA comprising a nucleotide sequence that is 100% identical to the nucleotide sequence of SEQ ID NO: 2, wherein 100% of the uridines of the mRNA are modified or replaced with N1-methylpseudouridine.

In some embodiments, the disclosure provides an mRNA comprising a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of ID NO: 14, wherein 100% of the uridines of the mRNA are modified or replaced with N1-methylpseudouridine.

In some embodiments, the disclosure provides an mRNA comprising a nucleotide sequence that is 100% identical to the nucleotide sequence of SEQ ID NO: 14, wherein 100% of the uridines of the mRNA are modified or replaced with N1-methylpseudouridine.

In some embodiments, any of the foregoing mRNA further comprises a 5′ cap, such as one described herein. In some embodiments, the 5′ cap is a cap-0, a cap-1, or a cap-2 structure.

### Systems for Genome Editing

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g., Cas nuclease) in combination with a guide RNA (gRNA) into a cell, resulting in a DNA cleavage event (e.g., the formation a double-strand break (DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g. genes).

Site-directed polypeptides can introduce DSBs in nucleic acids, e.g., genomic DNA. The DSB can stimulate a cell's endogenous DNA-repair pathways. Non-homologous end joining (NHEJ) can repair a DSB without the need for a homologous template. This can result in small deletions or insertions (indels) in the target nucleic acid at the site of cleavage, and can lead to disruption or alteration of gene expression. Homology-dependent repair (HDR) can occur when a homologous repair template, or exogenous donor template, is available. The homologous donor template comprises sequences that are homologous to sequences flanking the target nucleic acid cleavage site. For the purposes of genome editing, the repair template can be supplied as an exogenous nucleic acid. An exogenous nucleic acid is termed a donor polynucleotide (or donor template, or donor, or donor sequence) herein. With donor polynucleotides, an additional nucleic acid sequence (such as a transgene) or modification (such as a single or multiple base change or a deletion) can be introduced at the cleavage site so that the additional or altered nucleic acid sequence also becomes incorporated into the target locus.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. Mutations contemplated include substitutions, additions, and deletions, or any combination thereof.

The processes of deleting genomic DNA and integrating non-native nucleic acid into genomic DNA are examples of genome editing.

### I. Guide RNAs

In some embodiments, the disclosure provides a system for genome editing comprising at least one guide RNA (gRNA) molecule, which interacts with a site-directed endonuclease, such as a Cas nuclease (e.g., a SpCas9 polypeptide) to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g. a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA: tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA provided by the disclosure comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the tracrRNA may comprise all or a portion of a wild-type tracrRNA sequence (e.g., a tracrRNA from S. pyogenes). In some embodiments, the tracrRNA may comprise a truncated or modified variant of the wild-type tracr RNA. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA comprises certain secondary structures, such as, e.g., one or more hairpins or stem-loop structures, or one or more bulge structures.

### A. Single Guide RNAs

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., SpCas9), guide the Cas nuclease to a target sequence and activate the Cas nuclease for cleavage the target nucleic acid (e.g., genomic DNA). Accordingly, in some embodiments, the gRNA comprises a crRNA and a tracrRNA that are operably linked. In some embodiments, the sgRNA comprises a crRNA covalently linked to a tracrRNA. In some embodiments, the crRNA and the tracrRNA is covalently linked via a linker. In some embodiments, the sgRNA comprises a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

### B. Spacer Sequences

In some embodiments, the gRNAs of the disclosure comprise a spacer sequence. A spacer sequence is a sequence that defines the target site of a target nucleic acid (e.g., genomic DNA). The spacer sequence hybridizes to a target sequence in a target nucleic acid of interest. The spacer interacts with the target nucleic acid in a sequence-specific manner via hybridization (i.e., base pairing). The nucleotide sequence of the spacer can vary depending on the sequence of the target nucleic acid of interest. The spacer sequence is also referred to as the DNA-targeting segment.

The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. The gRNA spacer sequence hybridizes to the complementary strand (i.e., the non-PAM strand of the target nucleic acid/target site). In some embodiments, the spacer is sufficiently complementary to the complementary strand of the target sequence (i.e., non-PAM strand), as to target a site-directed endonuclease, such as a Cas nuclease (e.g., a SpCas9 polypeptide) to the target nucleic acid/target site.

In some embodiments, the spacer is at least 80%, 85%, 90% or 95% complementary to the non-PAM strand of the target nucleic acid. In some embodiments, the spacer is 100% complementary to the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the target sequence and the PAM comprises the formula 5′ N19-21-N-R-G-3′, wherein N is any nucleotide, and wherein R is a nucleotide comprising the nucleobase adenine (A) or guanine (G), and wherein the three 3′ terminal nucleic acids, N-R-G represent the S. pyogenes PAM. In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

In some embodiments, the spacer can include at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

### II. Donor Polynucleotides

The disclosure provides donor polynucleotides that, upon insertion into a DSB, correct or induce a mutation in a target nucleic acid (e.g., a genomic DNA). In some embodiments, the donor polynucleotides provided by the disclosure are recognized and used by the HDR machinery of a cell to repair a double strand break (DSB) introduced into a target nucleic acid by a site-directed nuclease, wherein repair of the DSB results in the insertion of the donor polynucleotide into the target nucleic acid. Alternatively, a donor polynucleotide may have no regions of homology to the targeted location in the DNA and may be integrated by NHEJ-dependent end joining following cleavage at the target site.

A donor polynucleotide or template can be single-stranded and/or double-stranded DNA, and can be introduced into a cell in linear or circular form. In some embodiments, the donor polynucleotide can be a double-stranded oligonucleotide (dsODNs) or a single-stranded oligonucleotide (ssODN). If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter at the integration site, namely the promoter that drives expression of the endogenous gene into which the donor is inserted. However, in some embodiments, the donor template comprises an exogenous promoter and/or enhancer, for example a constitutive promoter, an inducible promoter, or tissue-specific promoter.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, the donor polynucleotides comprise a nucleotide sequence which corrects or induces a mutation in a genomic DNA (gDNA) molecule in a cell, wherein when the donor polynucleotide is introduced into the cell in combination with a site-directed nuclease, a HDR or NHEJ DNA repair pathway inserts the donor polynucleotide into a double-stranded DNA break (DSB) introduced into the gDNA by the Cas9 nuclease (e.g., SpCas9 polypeptide) at a location proximal to the mutation, thereby correcting the mutation.

In some embodiments, the donor polynucleotide comprises a nucleotide sequence which corrects or induces a mutation, wherein the nucleotide sequence that corrects or induces a mutation comprises a single or multiple nucleotide(s). In some embodiments, the nucleotide sequence which corrects or induces a mutation comprises one or more codon(s). In some embodiments, the nucleotide sequence which corrects or induces a mutation comprises an exonic and/or intronic sequence.

In some embodiments, repair of the target nucleic acid molecule with the donor polynucleotide results in an insertion, deletion, or substitution of one or more nucleotides of the target nucleic acid molecule. In some embodiments, the insertion, deletion, or substitution of one or more nucleotides results in one or more amino acid changes in a protein expressed from a gene comprising the target sequence. In some embodiments, the insertion, deletion, or substitution of one or more nucleotides results in one or more nucleotide changes in an RNA expressed from the target gene. In some embodiments, the insertion, deletion, or substitution of one or more nucleotides alters the expression level of the target gene. In some embodiments, the insertion, deletion, or substitution of one or more nucleotides results in increased or decreased expression of the target gene. In some embodiments, the insertion, deletion, or substitution of one or more nucleotides results in gene knockdown. In some embodiments, the insertion, deletion, or substitution of one or more nucleotides results in gene knockout. In some embodiments, the repair of the target nucleic acid molecule with the donor polynucleotide results in replacement of an exon sequence, an intron sequence, a transcriptional control sequence, a translational control sequence, a sequence comprising a splicing signal, or a non-coding sequence of the target gene.

The donor polynucleotide is of a suitable length to correct or induce a mutation in a gDNA. In some embodiments, the donor polynucleotide comprises 10, 15, 20, 25, 50, 75, 100, 150, 200, 250, 300, 400, 500, or more nucleotides in length. In some embodiments, the donor polynucleotide is about 10-100, about 10-300, about 20-80, about 30-70, or about 40-60 nucleotides in length. In some embodiments, the donor polynucleotide is about 10-100 nucleotides in length. In some embodiments, the donor polynucleotide is about 20-80 nucleotides in length. In some embodiments, the donor polynucleotide is about 30-70 nucleotides in length. In some embodiments, the donor polynucleotide is about 40-60 nucleotides in length. In some embodiments, the donor polynucleotide is about 10-200 nucleotides in length. In some embodiments, the donor polynucleotide is about 10-500 nucleotides in length.

In some embodiments, for example those described herein wherein a donor polynucleotide is incorporated into the cleaved target site by a NHEJ DNA repair pathway, the donor polynucleotide has no homology arms. In some embodiments, for example those described herein wherein a donor polynucleotide is incorporated into the cleaved target site by an HDR DNA repair pathway, the donor polynucleotide has a left homology arm, a right homology arm, or both. In some embodiments, the left homology arm, the right homology arm, or both are of sufficient length (e.g., 100-1000s nucleotides in length) to facilitate insertion of the exogenous nucleotide sequence by an HDR DNA repair pathway.

The donor polynucleotides provided by the disclosure are produced by suitable nucleic acid synthesis method or means known in the art, such as those described in more detail below. DNA synthesis is the natural or artificial creation of deoxyribonucleic acid (DNA) molecules. The term DNA synthesis refers to DNA replication, DNA biosynthesis (e.g., in vivo DNA amplification), enzymatic DNA synthesis (e.g., polymerase chain reaction (PCR); in vitro DNA amplification) or chemical DNA synthesis.

In some embodiments, each strand of the donor polynucleotide is produced by oligonucleotide synthesis. Oligonucleotide synthesis is the chemical synthesis of relatively short fragments or strands of single-stranded nucleic acids with a defined chemical structure (sequence). Methods of oligonucleotide synthesis are known in the art (see e.g., Reese (2005) Organic & Biomolecular Chemistry 3 (21): 3851). The two strands can then be annealed together or duplexed to form a donor polynucleotide.

In some embodiments, donor polynucleotides are provided with chemistries suitable for delivery and stability within cells. Furthermore, in some embodiments, chemistries are provided that are useful for controlling the pharmacokinetics, biodistribution, bioavailability and/or efficacy of the donor polynucleotides described herein. Accordingly, in some embodiments, donor polynucleotides described herein may be modified, e.g., comprise a modified sugar moiety, a modified internucleoside linkage, a modified nucleoside, a modified nucleotide, and/or combinations thereof. In addition, the modified donor polynucleotides may exhibit one or more of the following properties: are not immune stimulatory; are nuclease resistant; have improved cell uptake compared to unmodified donor polynucleotides; and/or are not toxic to cells or mammals.

Nucleotide and nucleoside modifications have been shown to make a polynucleotide (e.g., a donor polynucleotide) into which they are incorporated more resistant to nuclease digestion than the native polynucleotide and these modified polynucleotides have been shown to survive intact for a longer time than unmodified polynucleotides. Specific examples of modified oligonucleotides include those comprising modified backbones (i.e. modified internucleoside linkage), for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see e.g., De Mesmaeker et al., Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the polynucleotide is replaced with a polyamide backbone, the nucleotides being bound directly or indirectly to the aza nitrogen atoms of the polyamide backbone, see Nielsen et al., Science 1991, 254, 1497). Phosphorus-containing modified linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Cyclohexenyl nucleic acid oligonucleotide mimetics are described in Wang et al., J. Am. Chem. Soc, 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments, the donor polynucleotides of the disclosure are stabilized against nucleolytic degradation such as by the incorporation of a modification (e.g., a nucleotide modification). In some embodiments, donor polynucleotides of the disclosure include a phosphorothioate at least the first, second, and/or third internucleotide linkage at the 5′ and/or 3′ end of the nucleotide sequence. In some embodiments, donor polynucleotides of the disclosure include one or more 2′-modified nucleotides, e.g., 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In some embodiments, donor polynucleotides of the disclosure include a phosphorothioate and a 2′-modified nucleotide as described herein.

Any of the modified chemistries described herein can be combined with each other, and that one, two, three, four, five, or more different types of modifications can be included within the same molecule. In some embodiments, the donor polynucleotide comprises 1, 2, 3, 4, 5, 6, 7, 8, 9, 10 or modifications.

In some aspects, the insertion of a donor polynucleotide into a DSB is determined by a suitable method known in the art. For example, after the insertional event, the nucleotide sequence of PCR amplicons generated using PCR primer that flank the DSB site is analyzed for the presence of the nucleotide sequence comprising the donor polynucleotide. Next-generation sequencing (NGS) techniques are used to determine the extent of donor polynucleotide insertion into a DSB analyzing PCR amplicons for the presence or absence of the donor polynucleotide sequence. Further, since each donor polynucleotide is a linear, dsDNA molecule, which can insert in either of two orientations, NGS analysis can be used to determine the extent of insertion of the donor polynucleotide in either direction.

In some aspects, the insertion of the donor polynucleotide and its ability to correct a mutation is determined by nucleotide sequence analysis of mRNA transcribed from the gDNA into which the donor polynucleotide is inserted. An mRNA transcribed from gDNA containing an inserted donor polynucleotide is analyzed by a suitable method known in the art. For example, conversion of mRNA extracted from cells treated or contacted with a donor polynucleotide or system provided by the disclosure is enzymatically converted into cDNA, which is further by analyzed by NGS analysis to determine the extent of mRNA molecule comprising the corrected mutation.

In other aspects, the insertion of a donor polynucleotide and its ability to correct a mutation is determined by protein sequence analysis of a polypeptide translated from an mRNA transcribed from the gDNA into which the donor polynucleotide is inserted. In some embodiments, a donor polynucleotide corrects or induces a mutation by the incorporation of a codon into an exon that makes an amino acid change in a gene comprising a gDNA molecule, wherein translation of an mRNA from the gene containing the inserted donor polynucleotide generates a polypeptide comprising the amino acid change. The amino acid change in the polypeptide is determined by protein sequence analysis using techniques including, but not limited to, Sanger sequencing, mass spectrometry, functional assays that measure an enzymatic activity of the polypeptide, or immunoblotting using an antibody reactive to the amino acid change.

III. Target sites

In some embodiments, the Cas nucleases (e.g., SpCas9 polypeptide) described herein are directed to and cleave (e.g., introduce a DSB) in a target nucleic acid molecule (e.g., genomic DNA). In some embodiments, a Cas nuclease (e.g., SpCas9 polypeptide) is directed by a gRNA to a target site of a target nucleic acid molecule (e.g., gDNA), where the guide RNA hybridizes with the complementary strand of the target sequence and the Cas nuclease cleaves the target nucleic acid at the target site. In some embodiments, the complementary strand of the target sequence is fully or partially complementary to the targeting sequence (e.g., spacer sequence) of the guide RNA.

In some embodiments, the target sequence comprises 18-24 nucleotides in length. In some embodiments, the target sequence comprises 19-21 nucleotides in length. In some embodiments, the target sequence comprises 20 nucleotides in length.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In other embodiments, the cell is a eukaryotic cell. In some embodiments, the eukaryotic cell is a mammalian cell. In some embodiments, the eukaryotic cell is a rodent cell. In some embodiments, the eukaryotic cell is a non-human primate cell. In some embodiments, the eukaryotic cell is a human cell. In some embodiments, the target sequence is a viral sequence. In some embodiments, the target sequence is a synthesized sequence. In some embodiments, the target sequence is on a eukaryotic chromosome, such as a human chromosome.

In some embodiments, the target sequence is located in a coding sequence of a gene, an intron sequence of a gene, a transcriptional control sequence of a gene, a translational control sequence of a gene, or a non-coding sequence between genes. In some embodiments, the gene is a protein coding gene. In other embodiments, the gene is a non-coding RNA gene. In some embodiments, the target sequence comprises all or a portion of a disease-associated gene. In some embodiments, the target sequence is located in a human gene selected from: transferrin (TF), albumin (ALB), serpin family A member 1 (SERPINA1), glucose-6-phosphatase catalytic subunit (G6PC), proprotein convertase subtilisin/kexin type 9 (PCSK9), alanine glyoxylate aminotransferase (AGXT), rhodopsin (RHO), guanylate cyclase 2D (GUCY2D), usherin (USH2A), and recombination activating 1 (RAGI).

In some embodiments, the target sequence is located in a non-genic functional site in the genome that controls aspects of chromatin organization, such as a scaffold site or locus control region. In some embodiments, the target sequence is a genetic safe harbor site, i.e., a locus that facilitates safe genetic modification.

In some embodiments, the target sequence is adjacent to a protospacer adjacent motif (PAM), a short sequence recognized by a CRISPR/Cas9 complex. In the PAM sequence comprises NGG (wherein N is defined as any nucleotide). In some embodiments, the PAM sequence is NGG.

### IV. Vectors

In some embodiments, the donor polynucleotide is provided by a vector. In some embodiments, the vector may be a DNA vector. In some embodiments, the vector may be circular. In other embodiments, the vector may be linear. Non-limiting exemplary vectors include plasmids, phagemids, cosmids, artificial chromosomes, minichromosomes, transposons, viral vectors, and expression vectors.

In some embodiments, the vector is a recombinant adeno-associated virus (AAV) vector. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692. See Table 1.

TABLE 1

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

In some embodiments, the method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

AAV vector serotypes can be matched to target cell types. For example, the following exemplary cell types can be transduced by the indicated AAV serotypes among others. See Table 2.

TABLE 2

| Tissue/Cell Type | Serotype |
|---|---|
| Liver | AAV3, AAV5, AAV8, AAV9 |
| Skeletal muscle | AAV1, AAV7, AAV6, AAV8, AAV9 |
| Central nervous system | AAV5, AAV1, AAV4, AAV8, AAV9 |
| RPE | AAV5, AAV4, AAV2, AAV8, AAV9, |
| | AAVrh8R |
| Photoreceptor cells | AAV5, AAV8, AAV9, AAVrh8R |
| Lung | AAV9, AAV5 |
| Heart | AAV8 |
| Pancreas | AAV8 |
| Kidney | AAV2, AAV8 |

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

### Nucleic Acid Modifications

In some embodiments, a nucleic acid of the disclosure (e.g., gRNA and/or mRNA) comprises one or more modified nucleobases, nucleosides, nucleotides or internucleoside linkages. In some embodiments, modified nucleic acids disclosure (e.g., gRNA and/or mRNA) have useful properties, including enhanced stability, intracellular retention, enhanced translation, and/or the lack of a substantial induction of the innate immune response of a cell into which the nucleic acid is introduced, as compared to a reference unmodified nucleic acid. Therefore, use of modified nucleic acids (e.g., gRNA and/or mRNA) may enhance the efficiency of protein production (e.g., SpCas9 expression from an mRNA of the disclosure), intracellular retention of the nucleic acids, efficiency of a genome editing system comprising the nucleic acid, as well as possess reduced immunogenicity.

In some embodiments, a gRNA and/or mRNA of the disclosure comprises one or more (e.g., 1, 2, 3, 4, 5, 6, 7, 8, 9, 10, 20, 30, 40, 50, 60, 70, 80, 90, 100, or more) different modified nucleobases, nucleosides, nucleotides or internucleoside linkages. In some embodiments, the modified nucleic acid (e.g., gRNA, and/or mRNA) has reduced degradation in a cell into which the nucleic acid is introduced, relative to a corresponding unmodified nucleic acid.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m3U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m5U, i.e., having the nucleobase deoxythymine), 1-methyl-pseudouridine (mw), 5-methyl-2-thio-uridine (m⁵s²U), 1-methyl-4-thio-pseudouridine (m¹s⁴ψ), 4-thio-1-methyl-pseudouridine, 3-methyl-pseudouridine (m3w), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m3Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include «-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (O₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2.7}G), N2, N2,7-dimethyl-guanosine (m^{2.2.7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2.7}Gm), 2′-O-methyl-inosine (Im), 1,2′-O-dimethyl-inosine (m¹Im), 2′-O-ribosylguanosine (phosphate) (Gr (p)), 1-thio-guanosine, O6-methyl-guanosine, 2′-F-ara-guanosine, and 2′-F-guanosine.

In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In certain embodiments, a nucleic acid (e.g., mRNA and/or gRNA) of the disclosure is uniformly modified (i.e., fully modified, modified through-out the entire sequence) for a particular modification. For example, an mRNA can be uniformly modified with N1-methylpseudouridine (m¹ψ) or 5-methyl-cytidine (m⁵C), meaning that all uridines or all cytosine nucleosides in the mRNA sequence are replaced with N1-methylpseudouridine (m¹ψ) or 5-methyl-cytidine (m⁵C). Similarly, a nucleic acid (e.g., mRNA and/or gRNA) of the disclosure can be uniformly modified for any type of nucleoside residue present in the sequence by replacement with a modified residue such as those set forth above.

### Methods of Making Nucleic Acids of the Disclosure

The nucleic acids (e.g., mRNA and/or gRNA) of the disclosure are produced by any suitable means available in the art, including but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized.

In some embodiments, one or more nucleic acids (e.g., mRNA and/or gRNA) of the disclosure are synthesized by enzymatic methods (e.g., in vitro transcription, IVT). In some embodiments, one or more nucleic acids (e.g., mRNA and/or gRNA) of the disclosure are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors that are used to in vitro transcribe a nucleic acid described herein.

In some aspects, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1 (3), 165-187 (1990).

In some embodiments, one or more nucleic acids (e.g., mRNA and/or gRNA) of the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, one or more nucleic acid modifications, such as those described herein, are introduced during or after chemical synthesis and/or enzymatic generation of the nucleic acids, e.g., modifications that enhance stability, reduce the likelihood or degree of innate immune response, and/or enhance other attributes, as described in the art.

In some embodiments, non-natural modified nucleobases are introduced into a nucleic acid (e.g., mRNA and/or gRNA) of the disclosure, during synthesis or post-synthesis. In certain embodiments, modifications are on internucleoside linkages, purine or pyrimidine bases, or sugar. In particular embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

### Nanoparticle Compositions

In some aspects, the disclosure provides nanoparticle compositions (e.g., lipid nanoparticles, LNPs) comprising an mRNA, one or more gRNAs, a donor polynucleotide, a system, or components of a system described herein. The mRNA, one or more gRNAs, a donor polynucleotide, a system, or components of a system may be formulated, individually or combined together, in nanoparticles or other delivery vehicles, (e.g., polymeric nanoparticles) to facilitate cellular uptake and/or to protect them from degradation when delivered to a subject (e.g., a patient with a mutation).

In some embodiments, a nanoparticle composition comprises a lipid. Lipid nanoparticles include, but are not limited to, liposomes and micelles. Any of a number of lipids may be present, including cationic and/or ionizable lipids, anionic lipids, neutral lipids, amphipathic lipids, conjugated lipids (e.g., PEGylated lipids), and/or structural lipids. Such lipids can be used alone or in combination.

Nanoparticles are ultrafine particles typically ranging between 1 and 100 to 500 nanometers (nm) in size with a surrounding interfacial layer and often exhibiting a size-related or size-dependent property. Nanoparticle compositions are myriad and encompass lipid nanoparticles (LNPs), liposomes (e.g., lipid vesicles), and lipoplexes. For example, a nanoparticle composition can be a liposome having a lipid bilayer with a diameter of 500 nm or less. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

As used herein, “size” or “mean size” in the context of nanoparticle compositions refers to the mean diameter of a nanoparticle composition.

In one embodiment, the polynucleotide encoding a polypeptide of interest are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

In some embodiments, the pKa of the nanoparticle is about 5-8. In some embodiments, the pKa of the nanoparticle is about 5. In some embodiments, the pKa of the nanoparticle is about 6. In some embodiments, the pKa of the nanoparticle is about 7. In some embodiments, the pKa of the nanoparticle is about 8.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

In some embodiments, the nanoparticle composition comprises a site-directed endonuclease mRNA, such as a SpCas9 mRNA, and gRNAs targeting one or more target sequences. In some embodiments, the SpCas9 mRNA and gRNAs are each separately formulated for delivery, e.g., in lipid nanoparticles. In some embodiments, the SpCas9 mRNA and gRNAs are co-formulated for delivery, e.g., in a lipid nanoparticle. In some embodiments, the nanoparticle composition further comprises a donor polynucleotide, either formulated individually or co-formulated with one or more components of the CRISPR/Cas system.

In some aspects, the disclosure provides lipid-based nanoparticle (LNP) compositions comprising: (a) one or more nucleic acid molecules (e.g., mRNA, gRNA, and/or donor polynucleotide) described herein; and (b) one or more lipid moieties selected from the group consisting of amino lipids, helper lipids, structural lipids, phospholipids, ionizable lipids, PEG lipids, lipoid, and cholesterol or cholesterol derivatives. In some aspects, the disclosure provides lipid-based nanoparticle (LNP) compositions comprising: (a) one or more nucleic acid molecules (e.g., mRNA, gRNA, and/or donor polynucleotide) described herein; and (b) one or more lipid moieties selected from the group consisting of ionizable lipids, amino lipids, anionic lipids, neutral lipids, amphipathic lipids, helper lipids, structural lipids, PEG lipids, and lipoids, and optionally (c) targeting moieties.

### I. LNP Components

A. Ionizable lipids

In some embodiments, the LNP composition disclosed herein comprises one or more one or more ionizable lipids. As used herein, the term “ionizable lipid” has its ordinary meaning in the art and may refer to a lipid comprising one or more charged moieties. In some embodiments, an ionizable lipid may be positively charged or negatively charged. In principle, there are no specific limitations concerning the ionizable lipids of the LNP compositions disclosed herein. In some embodiments, the one or more ionizable lipids are selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3ü)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3u)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3ü)-cholest-5-en-3-yloxy]octyl}oxy)-N,N-dimethyl-3-[(9Z,12Z)-octadeca-9,12-dien-1-y loxy]propan-1-amine (Octyl-CLinDMA (2S)). In one embodiment, the ionizable lipid may be selected from, but not limited to, an ionizable lipid described in International Publication Nos. WO2013086354 and WO2013116126.

In some embodiments, the lipid nanoparticle may include one or more (e.g., 1, 2, 3, 4, 5, 6, 7, or 8) cationic and/or ionizable lipids. Such cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 2-({8-[(3)-cholest-5-en-3-yloxy]octyl}oxy)-N,N-dimethyl-3-[(9Z, 12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2R)), (2S)-2-({8-[(3)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2S)).N,N-dioleyl-N,N-dimethylammonium chloride (“DODAC”); N-(2,3-dioleyloxy) propyl-N,N-N-triethylammonium chloride (“DOTMA”); N,N-distearyl-N,N-dimethylammonium bromide (“DDAB”); N-(2,3-dioleoyloxy) propyl)-N,N,N-trimethylammonium chloride (“DOTAP”); 1,2-Dioleyloxy-3-trimethylaminopropane chloride salt (“DOTAP.CI”); 3-β-(N-(N′,N′-dimethylaminoethane)-carbamoyl) cholesterol (“DC-Chol”), N-(1-(2,3-dioleyloxy) propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy) propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN® (including DOTMA and DOPE, available from GIBCO/BRL), and LIPOFECTAMINE® (including DOSPA and DOPE, available from GIBCO/BRL). KL10, KL22, and KL25 are described, for example, in U.S. Pat. No. 8,691,750.

### B. Amino Lipids

In some embodiments, the LNP composition disclosed herein comprise one or more amino lipids. The terms “amino lipid” and “cationic lipid” are used interchangeably herein to include those lipids and salts thereof having one, two, three, or more fatty acid or fatty alkyl chains and a pH-titratable amino head group (e.g., an alkylamino or dialkylamino head group). In principle, there are no specific limitations concerning the amino lipids of the LNP compositions disclosed herein. The cationic lipid is typically protonated (i.e., positively charged) at a pH below the pKa of the cationic lipid and is substantially neutral at a pH above the pKa. The cationic lipids can also be termed titratable cationic lipids. In some embodiments, the one or more cationic lipids include: a protonatable tertiary amine (e.g., pH-titratable) head group; alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DOTMA, DLinDMA, DLenDMA, γ-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2-DMA, C12-200, cKK-E12, cKK-A12, cKK-012, DLin-MC2-DMA (also known as MC2), and DLin-MC3-DMA (also known as MC3).

### C. Anionic Lipids

Anionic lipids suitable for use in lipid nanoparticles include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

### D. Neutral Lipids

Neutral lipids (including both uncharged and zwitterionic lipids) suitable for use in lipid nanoparticles include, but are not limited to, diacylphosphatidylcholine, diacylphosphatidylethanolamine, ceramide, sphingomyelin, dihydrosphingomyelin, cephalin, sterols (e.g., cholesterol) and cerebrosides. In some embodiments, the lipid nanoparticle comprises cholesterol. Lipids having a variety of acyl chain groups of varying chain length and degree of saturation are available or may be isolated or synthesized by well-known techniques. Additionally, lipids having mixtures of saturated and unsaturated fatty acid chains and cyclic regions can be used. In some embodiments, the neutral lipids used in the disclosure are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

### E. Amphipathic Lipids

In some embodiments, amphipathic lipids are included in nanoparticles. Exemplary amphipathic lipids suitable for use in nanoparticles include, but are not limited to, sphingolipids, phospholipids, fatty acids, and amino lipids.

The lipid composition of the pharmaceutical composition disclosed herein can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural amphipathic lipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the LNP composition disclosed herein comprises one or more phospholipids. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16:0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any mixtures thereof.

Other phosphorus-lacking compounds, such as sphingolipids, glycosphingolipid families, diacylglycerols, and β-acyloxyacids, may also be used. Additionally, such amphipathic lipids can be readily mixed with other lipids, such as triglycerides and sterols.

### F. Helper Lipids

In some embodiments, the LNP composition disclosed herein comprise one or more helper lipids. The term “helper lipid” as used herein refers to lipids that enhance transfection (e.g., transfection of an LNP comprising an mRNA that encodes a site-directed endonuclease, such as a SpCas9 polypeptide). In principle, there are no specific limitations concerning the helper lipids of the LNP compositions disclosed herein. Without being bound to any particular theory, it is believed that the mechanism by which the helper lipid enhances transfection includes enhancing particle stability. In some embodiments, the helper lipid enhances membrane fusogenicity. Generally, the helper lipid of the LNP compositions disclosure herein can be any helper lipid known in the art. Non-limiting examples of helper lipids suitable for the compositions and methods include steroids, sterols, and alkyl resorcinols. Particularly helper lipids suitable for use in the present disclosure include, but are not limited to, saturated phosphatidylcholine (PC) such as distearoyl-PC (DSPC) and dipalymitoyl-PC (DPPC), dioleoylphosphatidylethanolamine (DOPE), 1,2-dilinoleoyl-sn-glycero-3-phosphocholine (DLPC), cholesterol, 5-heptadecylresorcinol, and cholesterol hemisuccinate. In some embodiments, the helper lipid of the LNP composition includes cholesterol.

### G. Structural Lipids

In some embodiments, the LNP composition disclosed herein comprises one or more structural lipids. As used herein, the term “structural lipid” refers to sterols and also to lipids containing sterol moieties. Without being bound to any particular theory, it is believed that the incorporation of structural lipids into the LNPs mitigates aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In some embodiments, the structural lipid is cholesterol. In certain embodiments, the structural lipid is an analog of cholesterol.

### H. PEG-Lipids

The lipid component of a lipid nanoparticle composition may include one or more molecules comprising polyethylene glycol, such as PEG or PEG-modified lipids. In some embodiments, the LNP composition disclosed herein comprise one or more polyethylene glycol (PEG) lipid. The term “PEG-lipid” refers to polyethylene glycol (PEG)-modified lipids. Such lipids are also referred to as PEGylated lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. For example, a PEG lipid can be PEG-c-DOMG, PEG-DMG, PEG-DLPE, PEG-DMPE, PEG-DPPC, or a PEG-DSPE lipid. In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the lipid moiety of the PEG-lipids includes those having lengths of from about C14 to about C22, preferably from about C14 to about C16. In some embodiments, a PEG moiety, for example a mPEG-NH2, has a size of about 1000, 2000, 5000, 10,000, 15,000 or 20,000 daltons. In some embodiment, the PEG-lipid is PEG2k-DMG. In some embodiments, the one or more PEG lipids of the LNP composition comprises PEG-DMPE. In some embodiments, the one or more PEG lipids of the LNP composition comprises PEG-DMG.

In some embodiments, an LNP composition comprise one or more nucleic acid molecules described herein. In some embodiments, the LNP composition comprises an mRNA described herein (e.g., an mRNA encoding a site-directed endonuclease, such as a SpCas9 polypeptide). In some embodiments, the LNP compositions comprise one or more gRNA molecules (e.g., one, two, three, or four unique gRNA molecules) described herein. In some embodiments, the LNP composition comprises a donor polynucleotide described herein. In some embodiments, the LNP composition comprises an mRNA described herein (e.g., an mRNA encoding a site-directed endonuclease, such as a SpCas9 polypeptide) and one or more gRNA molecules (e.g., one, two, three, or four unique gRNA molecules) described herein.

In some embodiments, the ratio between the lipid components and the nucleic acid molecules (e.g., mRNA, gRNA, and/or donor polynucleotide) of the LNP composition, e.g., the weight ratio, is sufficient for (i) formation of LNPs with desired characteristics, e.g., size, charge, and (ii) delivery of a sufficient dose of nucleic acid at a dose of the lipid component(s) that is tolerable for in vivo administration as readily ascertained by one of skill in the art.

### I. Targeting Moieties

In certain embodiments, it is desirable to target a nanoparticle, e.g., a lipid nanoparticle, using a targeting moiety that is specific to a cell type and/or tissue type. In some embodiments, a nanoparticle may be targeted to a particular cell, tissue, and/or organ using a targeting moiety. In particular embodiments, a nanoparticle comprises a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′) 2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42 (5): 439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237:99-108, 1995; DeFrees et al., Journal of the American Chemistry Society 118:6101-6104, 1996; Blume et al., Biochimica et Biophysica Acta 1149:180-184,1993; Klibanov et al., Journal of Liposome Research 2:321-334, 1992; U.S. Pat. No. 5,013,556; Zalipsky, Bioconjugate Chemistry 4:296-299, 1993; Zalipsky, FEBS Letters 353:71-74, 1994; Zalipsky, in Stealth Liposomes Chapter 9 (Lasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2:321-334, 1992; Kirpotin et al., FEBS Letters 388:115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte.

### J. Lipidoids

The lipid nanoparticles described herein may be lipidoid-based. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat. Biotechnol. 2008 26:561-569; Love et al., Proc Natl Acad Sci USA. 2010 107:1864-1869; Siegwart et al., Proc Natl Acad Sci USA. 2011 108:12996-3001)

According to the present invention, complexes, micelles, liposomes or particles (e.g. nanoparticles) can be prepared containing these lipidoids and therefore, result in an effective delivery of an mRNA or system as described herein, as determined by, for example, the expression and/or activity of the site-directed endonuclease encoded by the mRNA and/or the editing efficiency of the system, following the injection via localized and systemic routes of administration. Pharmaceutical compositions comprising lipidoid complexes can be administered by various means disclosed herein.

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see e.g., Akinc et al., Mol Ther. 2009 17:872-879), use of lipidoid oligonucleotides to deliver the formulation to other cells types including, but not limited to, endothelial cells, myeloid cells, and muscle cells may not be similarly size-limited.

In one aspect, effective delivery to myeloid cells, such as monocytes, lipidoid formulations may have a similar component molar ratio. Different ratios of lipidoids and other components including, but not limited to, a neutral lipid (e.g., diacylphosphatidylcholine), cholesterol, a PEGylated lipid (e.g., PEG-DMPE), and a fatty acid (e.g., an omega-3 fatty acid) may be used to optimize the formulation of the mRNA or system for delivery to different cell types including, but not limited to, hepatocytes, myeloid cells, muscle cells, etc. Exemplary lipidoids include, but are not limited to, DLin-DMA, DLin-K-DMA, DLin-KC2-DMA, 98N12-5, C12-200 (including variants and derivatives), DLin-MC3-DMA and analogs thereof. The use of lipidoid formulations for the localized delivery of nucleic acids to cells (such as, but not limited to, adipose cells and muscle cells) via either subcutaneous or intramuscular delivery, may also not require all of the formulation components which may be required for systemic delivery, and as such may comprise the lipidoid and the mRNA or system.

In a further embodiment, combinations of different lipidoids may be used to improve the efficacy of an mRNA or system described herein.

According to the present disclosure, an mRNA or system described herein may be formulated by mixing the mRNA or system, or individual components of the system, with the lipidoid at a set ratio prior to addition to cells. In vivo formulations may require the addition of extra ingredients to facilitate circulation throughout the body. After formation of the particle, a mRNA, system, or individual components of a system is added and allowed to integrate with the complex. The encapsulation efficiency is determined using a standard dye exclusion assays.

In vivo delivery of mRNA and/or systems may be affected by many parameters, including, but not limited to, the formulation composition, nature of particle PEGylation, degree of loading, oligonucleotide to lipid ratio, and biophysical parameters such as particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta [3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), MD1, DLin-DMA, DLin-K-DMA, DLin-KC2-DMA and DLin-MC3-DMA can be tested for in vivo activity. The lipidoid referred to herein as “98N12-5” is disclosed by Akinc et al., Mol Ther. 2009 17:872-879). The lipidoid referred to herein as “C12-200” is disclosed by Love et al., Proc Natl Acad Sci USA. 2010 107:1864-1869 and Liu and Huang, Molecular Therapy. 2010 669-670.

The ability of a lipidoid-formulated mRNA or system to alter a nucleotide sequence in a gDNA (e.g., correct or induce a mutation) in vitro or in vivo can be determined by any technique known in the art or described herein (e.g., next-generation DNA sequencing).

### K. Other Components

The nanoparticles disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

### II. Preparation of LNPs

The LNPs of the present disclosure, in which a nucleic acid described herein (e.g., mRNA, gRNA, and/or donor polynucleotide) is entrapped within the lipid portion of the particle and is protected from degradation, can be formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

In some embodiments, the LNPs of the present disclosure are produced via a continuous mixing method, e.g., a process that includes providing an aqueous solution a nucleic acid described herein (e.g., mRNA, gRNA, and/or donor polynucleotide) in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the nucleic acid molecule within the lipid vesicle. This process and the apparatus for carrying out this process are known in the art. More information in this regard can be found in, for example, U.S. Patent Publication No. 20040142025, the disclosure of which is herein incorporated by reference. The action of continuously introducing lipid and buffer solutions into a mixing environment, such as in a mixing chamber, causes a continuous dilution of the lipid solution with the buffer solution, thereby producing a lipid vesicle substantially instantaneously upon mixing. By mixing the aqueous solution comprising a nucleic acid molecule with the organic lipid solution, the organic lipid solution undergoes a continuous stepwise dilution in the presence of the buffer solution (e.g., aqueous solution) to produce a nucleic acid-lipid particle.

In some embodiments, the LNPs of the present disclosure are produced via a direct dilution process that includes forming a lipid vesicle (e.g., liposome) solution and immediately and directly introducing the lipid vesicle solution into a collection vessel containing a controlled amount of dilution buffer. In some embodiments, the collection vessel includes one or more elements configured to stir the contents of the collection vessel to facilitate dilution. In some embodiments, the amount of dilution buffer present in the collection vessel is substantially equal to the volume of lipid vesicle solution introduced thereto.

In some embodiments, the LNPs of the present disclosure are produced via an in-line dilution process in which a third reservoir containing dilution buffer is fluidly coupled to a second mixing region. In these embodiments, the lipid vesicle (e.g., liposome) solution formed in a first mixing region is immediately and directly mixed with dilution buffer in the second mixing region. These processes and the apparatuses for carrying out direct dilution and in-line dilution processes are known in the art. More information in this regard can be found in, for example, U.S. Patent Publication No. 20070042031, the disclosure of which is herein incorporated by reference.

### Pharmaceutical Compositions

The present disclosure includes pharmaceutical compositions comprising an mRNA, one or more gRNAs, and/or a donor polynucleotide described herein, in combination with one or more pharmaceutically acceptable excipient, carrier or diluent.

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, and range from a pH of about 3 to a pH of about 11, about pH 3 to about pH 7, depending on the formulation and route of administration. In alternative examples, the pH can be adjusted to a range from about pH 5.0 to about pH 8. In some examples, the compositions comprise a therapeutically effective amount of at least one compound as described herein, together with one or more pharmaceutically acceptable excipients.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Pharmaceutical compositions can be formulated into preparations in solid, semi-solid, liquid or gaseous forms, such as tablets, capsules, powders, granules, ointments, solutions, suppositories, injections, inhalants, gels, microspheres, and aerosols. As such, administration of a gRNA and/or mRNA and/or donor polynucleotide described herein can be achieved in various ways, including oral, buccal, rectal, parenteral, intraperitoneal, intradermal, transdermal, intratracheal, intraocular, etc., administration. The active agent can be systemic after administration or can be localized using regional administration, intramural administration, or use of an implant that acts to retain the active dose at the site of implantation. The active agent can be formulated for immediate activity or it can be formulated for sustained release.

In some cases, the components of the composition are individually pure, e.g., each of the components is at least about 75%, at least about 80%, at least about 90%, at least about 95%, at least about 98%, at least about 99%, or at least 99%, pure. In some cases, the individual components of a composition are pure before being added to the composition.

In some embodiments, the mRNA, one or more gRNA, and/or donor polynucleotides is encapsulated in a nanoparticle, e.g., a lipid nanoparticle. In some embodiments, the gRNA is encapsulated in a nanoparticle, e.g., a lipid nanoparticle. In some embodiments, the mRNA encoding a Cas nuclease (e.g. SpCas9) described herein is encapsulated in a nanoparticle, e.g., lipid nanoparticle. In particular embodiments, an mRNA encoding a Cas nuclease (e.g., SpCas9 polypeptide) described herein or lipid nanoparticle encapsulating an mRNA described herein is present in a pharmaceutical composition.

In particular embodiments, the mRNA encoding the site-directed endonuclease or nanoparticle encapsulating the mRNA encoding the site-directed endonuclease is present in a pharmaceutical composition. In various embodiments, the mRNA present in the pharmaceutical composition is encapsulated in a nanoparticle, e.g., a lipid nanoparticle. In some embodiments, the mRNA encoding the site-directed endonuclease and gRNA is encapsulated in a nanoparticle. In some embodiments, the mRNA encoding the site-directed endonuclease and gRNA is encapsulated in a nanoparticle at a mRNA: gRNA weight ratio of about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1 (wt/wt). In some embodiments, the mRNA: gRNA weight ratio is about 1:1.

In one embodiment, the lipid nanoparticles can comprise polynucleotides (e.g., donor polynucleotide) in a lipid: polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles can comprise the donor polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

Typically, an effective amount of a CRISPR/Cas system comprising an mRNA described herein, and/or one or more guide RNAs and/or donor polynucleotide can be provided. The amount of recombination can be measured by any convenient method, e.g. as described above, and known in the art. The calculation of the effective amount or effective dose of a CRISPR/Cas system comprising an mRNA described herein, and/or one or more guide RNAs and/or donor polynucleotide to be administered is within the skill of one of ordinary skill in the art, and can be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a CRISPR/Cas system comprising an mRNA described herein, and/or one or more guide RNA and/or donor polynucleotide can be obtained from a suitable commercial source. As a general proposition, the total pharmaceutically effective amount of a CRISPR/Cas system comprising an mRNA described herein, and/or one or more guide RNA and/or donor polynucleotide administered parenterally per dose will be in a range that can be measured by a dose response curve.

Therapies based on a CRISPR/Cas system comprising an mRNA described herein, and/or one or more guide RNA and/or donor polynucleotide to be used for therapeutic administration, must be sterile. Sterility is readily accomplished by filtration through sterile filtration membranes (e.g., 0.2 um membranes). Therapeutic compositions can be generally placed into a container having a sterile access port, for example, an intravenous solution bag or vial having a stopper pierceable by a hypodermic injection needle. The therapies based on a CRISPR/Cas system comprising an mRNA described herein, and/or one or more guide RNA and/or donor polynucleotide can be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution of compound, and the resulting mixture is lyophilized. The infusion solution can be prepared by reconstituting the lyophilized compound using bacteriostatic Water-for-Injection.

### Methods of Use

Provided herein are cellular, ex vivo and in vivo methods for using the CRISPR/Cas systems, the delivery systems, or pharmaceutical compositions provided herein, to create permanent changes in one or more target genes in the genome. Such methods use a Cas nuclease (e.g., SpCas9 polypeptide) encoded by an mRNA described herein, one or more gRNAs, and optionally a donor polynucleotide, to permanently delete (excise), insert, or replace (delete and insert) exons and/or introns in target genes in the genome.

In some aspects, the disclosure provides methods for inducing a double-stranded break (DSB) in a target gene in a cell. The method includes contacting the cell with: (i) the mRNA provided herein and at least one gRNA directed to the target gene; (ii) the system provided herein; or (iii) the pharmaceutical composition provided herein, wherein the mRNA is translated when the mRNA, the system, or the composition contacts the cell and provides a site-directed endonuclease that combines with the gRNA to induce a DSB at a site in the target gene.

In some aspects, the disclosure provides methods for correcting a mutation in a target gene in a cell. The method includes contacting the cell with the mRNA provided herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

In some aspects, the disclosure provides methods for treating, preventing, reducing the risk or likelihood of developing (e.g., reducing the susceptibility to), delaying the onset of, and/or ameliorating one or more symptoms associated with a health condition or a disease in a mammal (e.g., human) in need thereof, the method including administering to the mammal a therapeutically effective amount of an mRNA, a CRISPR/Cas system, a delivery system, or a pharmaceutical composition described herein.

In some embodiments, the cell is a eukaryotic cell. Non-limiting examples of eukaryotic cells include yeast cells, plant cells, insect cells, cells from an invertebrate animal, cells from a vertebrate animal, mammalian cells, rodent cells, mouse cells, rat cells, and human cells. In some embodiments, the eukaryotic cell may be a mammalian cell. In some embodiments, the eukaryotic cell may be a rodent cell. In some embodiments, the eukaryotic cell may be a non-human primate cell. In some embodiments, the eukaryotic cell may be a human cell. Similarly, the target sequence may be from any such cells or in any such cells.

The mRNA, system, or pharmaceutical composition described herein may be introduced into the cell via any methods known in the art, such as, e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, shear-driven cell permeation, fusion to a cell-penetrating peptide followed by cell contact, microinjection, and nanoparticle-mediated delivery. In some embodiments, the vector system may be introduced into the cell via viral infection.

In some aspects, the disclosure provides methods of treating a patient with a disease by inducing a DSB in a target gene in a cell. The method includes isolating a cell from the patient, and contacting the cell with: (i) the mRNA provided herein and at least one gRNA directed to the target gene; (ii) the system provided herein; or (iii) the pharmaceutical composition provided herein, wherein the mRNA is translated when the mRNA, system, or composition contacts the cell and provides a site-directed endonuclease that combines with the gRNA to induce a DSB at a site in the target gene.

In some aspects, the disclosure provides methods of treating a patient with a disease by inducing a DSB in a target gene in a cell. The method includes administering to the patient an effective amount of: (i) the mRNA provided herein and at least one gRNA directed to the target gene; (ii) the system provided herein; or (iii) the pharmaceutical composition provided herein, wherein the mRNA is translated when the mRNA, system, or composition contacts the cell and provides a site-directed endonuclease that combines with the gRNA to induce a DSB at a site in the target gene.

In some aspects, the disclosure provides methods of treating a patient with a disease by correcting a mutation in a target gene in a cell. The method includes isolating a cell from the patient; and contacting the cell with the mRNA provided herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

In some aspects, the disclosure provides methods of treating a patient with a disease by correcting a mutation in a target gene in a cell. The method includes administering to the patient an effective amount of the mRNA provided herein, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

As used herein, the terms “administration” and “administering,” refers to the delivery of an mRNA or CRISPR/Cas system described herein, e.g., via a delivery system described herein, or a pharmaceutical composition thereof, by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

In some aspects, the methods comprise administration of an mRNA, CRISPR/Cas system, delivery system, or pharmaceutical composition described herein, as an ex vivo cell-based therapy. For example, a patient specific iPS cell line is created. Then, the chromosomal DNA of these iPS cells is corrected using the materials and methods described herein. Next, the corrected iPSCs are differentiated. Finally, the progenitor cells are implanted into the patient. There are many advantages to this ex vivo approach. One advantage of an ex vivo cell therapy approach is the ability to conduct a comprehensive analysis of the therapeutic prior to administration. All nuclease-based therapeutics have some level of off-target effects. Performing gene correction ex vivo allows one to fully characterize the corrected cell population prior to implantation. Another advantage of ex vivo cell therapy relates to genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

In some aspects, the methods comprise administration of an mRNA, CRISPR/Cas system, delivery system, or pharmaceutical composition described herein, as an in vivo based therapy. In this method, the chromosomal DNA of the cells in the patient is corrected using the materials and methods described herein. An advantage of in vivo gene therapy is the ease of therapeutic production and administration.

In some embodiments, the methods comprise administering an mRNA, CRISPR/Cas system, delivery system, or pharmaceutical composition described herein to a patient in need thereof. In some embodiments, the method is used as a single therapy or in combination with other therapies known in the art.

In some embodiments, the patient may have a mutation (such as, e.g., insertion, deletion, substitution, chromosome translocation) in a disease-associated gene. In some embodiments, administration of the mRNA, CRISPR/Cas system, delivery system, or pharmaceutical composition may result in a mutation comprising an insertion, deletion, or substitution of one or more nucleotides of the disease-associated gene in the patient. Certain embodiments may include methods of repairing the patient's mutation in the disease-associated gene. In some embodiments, the mutation may result in one or more amino acid changes in a protein expressed from the disease-associated gene. In some embodiments, the mutation may result in one or more nucleotide changes in an RNA expressed from the disease-associated gene. In some embodiments, the mutation may alter the expression level of the disease-associated gene. In some embodiments, the mutation may result in increased or decreased expression of the gene. In some embodiments, the mutation may result in gene knockdown in the patient. In some embodiments, the administration of the mRNA, CRISPR/Cas system, delivery system, or pharmaceutical composition may result in the correction of the patient's mutation in the disease-associated gene. In some embodiments, the administration of the mRNA, CRISPR/Cas system, delivery system, or pharmaceutical composition may result in gene knockout in the patient. In some embodiments, the administration of the mRNA, CRISPR/Cas system, delivery system, or pharmaceutical composition may result in replacement of an exon sequence, an intron sequence, a transcriptional control sequence, a translational control sequence, or a non-coding sequence of the disease-associated gene.

In some embodiments, the administration of the mRNA, CRISPR/Cas system, delivery system, or pharmaceutical composition may result in integration of an exogenous sequence (e.g., a donor polynucleotide sequence) into the patient's genomic DNA. In some embodiments, the exogenous sequence may comprise a protein or RNA coding sequence operably linked to an exogenous promoter sequence such that, upon integration of the exogenous sequence into the patient's genomic DNA, the patient is capable of expressing the protein or RNA encoded by the integrated sequence. The exogenous sequence may provide a supplemental or replacement protein coding or non-coding sequence. For example, the administration of the mRNA, CRISPR/Cas system, delivery system, or pharmaceutical composition may result in the replacement of the mutant portion of the disease-associated gene in the patient. In some embodiments, the mutant portion may include an exon of the disease-associated gene. In other embodiments, the integration of the exogenous sequence may result in the expression of the integrated sequence from an endogenous promoter sequence present on the patient's genomic DNA. For example, the administration of the mRNA, CRISPR/Cas system, delivery system, or pharmaceutical composition may result in supply of a functional gene product of the disease-associated gene to rectify the patient's mutation. In yet other embodiments, the administration of the mRNA, CRISPR/Cas system, delivery system, or pharmaceutical composition may result in integration of an exon sequence, an intron sequence, a transcriptional control sequence, a translational control sequence, or a non-coding sequence into the patient's genomic DNA.

Additional embodiments of the invention also encompass methods of treating the patient in a tissue-specific manner. Non-limiting examples of suitable tissues for treatment by the methods include the immune system, neuron, muscle, pancreas, blood, kidney, bone, lung, skin, liver, and breast tissues.

### Kits

The present disclosure provides kits for carrying out the methods described herein. A kit can include an mRNA described herein, one or more gRNA, and optionally a donor polynucleotide, sufficient to carry out the aspects of the methods described herein. Components of a kit can be in separate containers, or combined in a single container.

In some embodiments, a kit comprises an mRNA described herein and instructions for use with one or more gRNAs, and optionally a donor polynucleotide, for editing a target gene in a cell. In some embodiments, the kit is for inducing a DSB in a target gene in a cell. The kit can include a container comprising an mRNA provided herein, the system provided herein, or the pharmaceutical composition provided herein, and a package insert comprising instructions for use.

Any kit described above can further comprise one or more additional reagents, where such additional reagents are selected from a buffer, a buffer for introducing a nucleic acid or delivery system described herein into a cell, a wash buffer, a control reagent, a control vector, a control RNA polynucleotide, a reagent for in vitro production of the polypeptide (e.g., SpCas9 polypeptide) from an mRNA described herein, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the SpCas9 polypeptide encoded by an mRNA described herein, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a,” “an,” and “the” include plural references unless the context clearly dictates otherwise. For example, the term “a cell” includes one or more cells, comprising mixtures thereof. “A and/or B” is used herein to include all of the following alternatives: “A,” “B,” “A or B,” and “A and B.”

The term “about,” as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

It is understood that aspects and embodiments of the disclosure described herein include “comprising,” “consisting,” and “consisting essentially of” aspects and embodiments.

The terms “individual,” “subject,” “host,” and “patient,” are used interchangeably herein and refer to any mammalian subject, such as human (e.g., human subjects), non-human mammals and non-human primates, for whom diagnosis, treatment, or therapy is desired, particularly humans.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein. In some embodiments, a nucleic acid molecule of the disclosure is an mRNA described herein, such as an mRNA encoding a site-directed endonuclease, such as a SpCas9 polypeptide described herein. In some embodiments, a nucleic acid molecule of the disclosure is a gRNA described herein. In some embodiments, a nucleic acid molecule of the disclosure is a donor polynucleotide described herein.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, or mafft.cbrc.jp/alignment/software/. See, e.g., Altschul et al. (1990), J. Mol. Biol. 215:403-10. Sequence alignments standard in the art are used according to the disclosure to determine nucleotides in an mRNA described herein that “correspond to” nucleotides in another mRNA. The nucleotides of the first mRNA that correspond to nucleotides of the second mRNA appear at the same position in alignments of the sequences.

A DNA sequence that “encodes” a particular RNA is a DNA nucleic acid sequence that is transcribed into RNA. A DNA polynucleotide can encode an RNA (mRNA) that is translated into protein, or a DNA polynucleotide can encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector (e.g., an AAV). As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

The term “operably linked,” as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements are contiguous or non-contiguous.

As used herein, the term “genomic DNA (gDNA)” refers to the DNA of a genome of an organism including, but not limited to, the DNA of the genome of a bacterium, fungus, archea, plant or animal.

As used herein, the term “manipulating” or “editing” DNA encompasses binding, or cleaving (i.e., cutting) one or both strands of the DNA, or encompasses modifying the DNA or a polypeptide associated with the DNA. Manipulating or editing DNA can silence, activate, or modulate (either increase or decrease) the expression of an RNA or polypeptide encoded by the DNA.

As used herein, the terms “nuclease” and “endonuclease” are used interchangeably herein to mean an enzyme which possesses endonucleolytic catalytic activity for polynucleotide cleavage. The term includes site-specific endonucleases such as site-specific endonucleases of clustered, regularly interspaced, short palindromic repeat (CRISPR) systems such as, e.g., Cas polypeptides, e.g., a SpCas9 polypeptide.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (e.g., spacer or spacer sequence) of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The target DNA can be a double-stranded DNA. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” The target DNA can be within a target gene.

By “site-directed endonuclease,” it is meant a polypeptide (e.g., Cas9 polypeptide, SpCas9 polypeptide) that binds gRNA and is targeted to a specific DNA sequence. A site-directed endonuclease as described herein is targeted to a specific DNA sequence by the RNA molecule (e.g., gRNA) to which it is bound. The RNA molecule comprises a sequence that binds, hybridizes to, or is complementary to a target sequence within the target DNA, thus targeting the bound polypeptide (e.g., Cas9 polypeptide, SpCas9 polypeptide) to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain aspects, a complex comprising a guide RNA and a site-directed modifying polypeptide is used for targeted double-stranded DNA cleavage.

As used herein, the term “SpCas9 polypeptide” refers to a Cas9 polypeptide derived from S. pyogenes. As used herein, the term “SpCas9 mRNA” refers to an mRNA encoding a SpCas9 polypeptide.

As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (e.g., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

As used herein, the term “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment,” “treating,” and the like are used herein to generally mean obtaining a desired pharmacologic and/or physiologic effect. The effect is prophylactic in terms of completely or partially preventing a disease or symptom thereof and/or is therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which is predisposed to acquiring the disease or symptom but has not yet been diagnosed as having it; (b) inhibiting the disease or symptom, e.g., arresting its development; or (c) relieving the disease, e.g., causing regression of the disease. The therapeutic agent is administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

### EXAMPLES

The present disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

The practice of the present disclosure will employ, unless otherwise indicated, techniques of molecular biology, microbiology, cell biology, biochemistry, nucleic acid chemistry, and immunology, which are known to those skilled in the art. Such techniques are explained in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F.M. Ausubel et al., eds., 1987, including supplements through 2014); PCR: The Polymerase Chain Reaction, (Mullis et al., eds., 1994); Beaucage et al. eds., Current Protocols in Nucleic Acid Chemistry, John Wiley & Sons, Inc., New York, 2000, (including supplements through 2014), Gene Transfer and Expression in Mammalian Cells (Makrides, ed., Elsevier Sciences B.V., Amsterdam, 2003); and Current Protocols in Immunology (Horgan K and S. Shaw (1994), including supplements through 2014). As appropriate, procedures involving the use of commercially available kits and reagents are generally carried out in accordance with manufacturer defined protocols and/or parameters unless otherwise noted.

### Example 1: Preparation of Optimized mRNA Encoding a Site-Specific Endonuclease

Messenger RNA (mRNA) encoding a site-directed endonuclease is a valuable tool for use in gene-editing systems. However, administration of exogenous mRNA can activate host innate immune response pathways, which is detrimental to mRNA expression and can result in a potentially toxic inflammatory response. Uridine-rich motifs present in exogenous mRNA have been shown to induce innate immune activation, for example, by stimulating RIG-1 (retinoic acid-inducible gene I) pattern recognition receptor (see, e.g., Chiang, et al (2015) J. VIROL. 89:8011; Runge, et al (2014) PLoS PATHOG. 10: e1004081; Saito, et al (2008) NATURE 454:523). Thus, it was evaluated if mRNA with uridine depletion and/or chemical modification of uridine would provide efficient translation of an encoded Cas9 site-directed endonuclease while reducing undesirable immune activation.

Towards this end, a parent mRNA (i.e., reference mRNA) for generating SpCas9 (SEQ ID NO: 6) was prepared. The parent mRNA included a synthetic 5′ UTR, an open-reading frame (ORF) encoding an N-terminal nuclear localization signal derived from SV40 (SEQ ID NO: 8), SpCas9 (SEQ ID NO: 6), and a C-terminal NLS derived from nucleoplasmin (SEQ ID NO: 7), a 3′ UTR, and a poly-A-tail. The parent mRNA was cloned into an mRNA expression vector, which contained a T7 RNA polymerase promoter. A transcription template was generated by PCR.

Sequence modified versions of the parent mRNA were prepared. This included “RNA-009”, which was prepared as a codon-optimized version of the parent mRNA using an algorithm designed by Atum (Newark, CA). Additionally, a uridine-depleted version of RNA-009 was prepared (“RNA-013”) using an algorithm designed by Geneious (San Diego, CA). Furthermore, “RNA-012” was prepared as a codon-optimized and uridine-depleted version of the parent mRNA using the Geneious algorithm

The nucleotide sequences of the parent mRNA and RNA-009 (including ORF, 5′ UTR, and 3′ UTR) are identified in Table 3.

TABLE 3
Sequences of mRNA encoding SpCas9

| | | | |
|---|---|---|---|
| | DNA SEQ ID | RNA SEQ ID | Amino Acid |
| Sequence Name | NO: | NO: | SEQ ID NO: |

Parent mRNA

| | | | |
|---|---|---|---|
| Full-length mRNA | 16 | 17 | — |

RNA-009 (Sequence Optimtzed from Parent mRNA)

| | | | |
|---|---|---|---|
| Full-length mRNA | 1 | 2 | — |
| 5′ UTR | 9 | 10 | — |
| Coding Region | 3 | 4 | 5 |
| 3′ UTR | 11 | 12 | — |
| Poly-A Tail | 13 | 13 | — |

The base content of the parent mRNA, RNA-009, RNA-012, and RNA-013 was quantified and provided in Table 4. While the RNA-009 had increased uridine content compared to the parent mRNA, RNA-012 and RNA-013 had reduced uridine content relative to both.

TABLE 4
Percent Nucleotide Base Content of SpCas9 mRNA

| Nucleotide | Parent mRNA | RNA-009 | RNA-012 | RNA-013 |
|---|---|---|---|---|
| A | 30.8 | 30.7 | 28.0 | 28.0 |
| C | 26.8 | 25.8 | 29.2 | 29.2 |
| G | 27.1 | 25.6 | 30.1 | 30.2 |
| U | 15.2 | 17.9 | 12.7 | 12.6 |

Additionally, the parent mRNA and RNA-009, RNA-012, and RNA-013 were aligned and sequence similarity was determined. As shown in Table 5, RNA-009 shared 84.3% sequence similarity with the parent mRNA, while the codon-optimized and uridine depleted RNA-012 and RNA-013 shared 99.6% sequence similarity (corresponding to a difference in approximately 16 nucleotides).

TABLE 5
Percent Pairwise Sequence Identity of SpCas9
mRNA Coding Regions

| Parent mRNA | RNA-009 | RNA-012 | RNA-013 | |
|---|---|---|---|---|
| 100 | 84.3 | 92.4 | 92.0 | Parent mRNA |
| 84.3 | 100 | 86.0 | 85.8 | RNA-009 |
| 92.4 | 86.0 | 100 | 99.6 | RNA-012 |
| 92.0 | 85.8 | 99.6 | 100 | RNA-013 |

Preparation of mRNA transcripts from parent mRNA, RNA-009, RNA-012, and RNA-013 mRNA constructs was performed using T7 RNA polymerase in vitro transcription (IVT) with co-transcriptional capping using a m⁷GpppG Cap-1. For unmodified mRNA, transcription was performed using a mixture of adenosine triphosphate (ATP), cytidine triphosphate (CTP), guanosine triphosphate (GTP), and uridine triphosphate (UTP). For chemically modified mRNA, transcription was performed with a UTP analog in place of UTP. The mRNA prepared are depicted in Table 6. The mRNA transcripts were purified using a combination of chromatographic techniques to enrich for full-length product and remove reaction byproducts, sequentially.

TABLE 6
Base Modifications of SpCas9 mRNA Constructs

| mRNA | Uridine Base Modification |
|---|---|
| RNA-009u | Unmodified |
| RNA-009n | N1-methylpseudouridine |
| RNA-009p | Pseudouridine |
| RNA-009m | 5-methoxyuridine |
| RNA-012u | Unmodified |
| RNA-012n | N1-methylpseudouridine |
| RNA-013n | N1-methylpseudouridine |

### Example 2: Evaluation of Optimized mRNA for Editing the Transferrin Gene Locus in Mice

In vivo editing efficiency and tolerability of sequence optimized SpCas9 RNA-009 (SEQ ID NO: 2) was evaluated in C57BL/6 mice. The RNA-009 transcripts contained either unmodified uridine (RNA-009u) or modified uridine (RNA-009m or RNA-009n) and were prepared as described in Example 1.

Editing of a target gene sequence in the transferrin gene locus was evaluated. For in vivo administration, the SpCas9 mRNA and mTF_T2 sgRNA were co-formulated as lipid nanoparticles (“RNA-LNP”) at a 1:1 mRNA: gRNA weight ratio. The LNPs were diluted in PBS to deliver the desired dose in a volume of 10 mL per kg of body weight.

C57BL/6 mice were administered the co-formulated RNA-LNP by intravenous tail vein injection at a dose of 0.4 mpk (N=4 per group). The dosage is expressed in mg of encapsulated RNA (mg of SpCas9 mRNA+mg of mTF_T2 sgRNA) administered per kg of body weight (“mpk”). Negative control mice were administered an equivalent volume of PBS by intravenous tail vein injection.

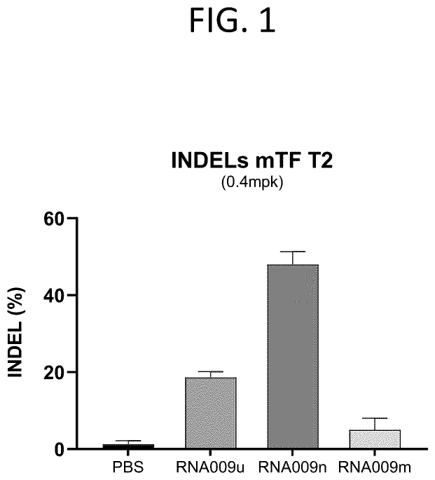

To evaluate editing efficiency, liver tissue was isolated from sacrificed mice at approximately 96 hours post-administration. The tissue was homogenized and genomic DNA was extracted using a Qiagen DNeasy Blood and Tissue Kit (Cat #69506) according to the manufacturer's protocol. The target sequence in the transferrin gene locus was amplified by PCR and sequenced. Indels were measured by TIDE analysis software as described by Brinkman et al., Nucleic Acids Res. 2014 Dec. 16; 42 (22): e168. As shown in FIG. 1, RNA-009 with N1-methylpseudouridine (RNA-009n) had improved editing efficiency compared to RNA-009 with unmodified uridine (RNA-009u) or RNA-009 with 5-methoxy uridine (RNA-009m).

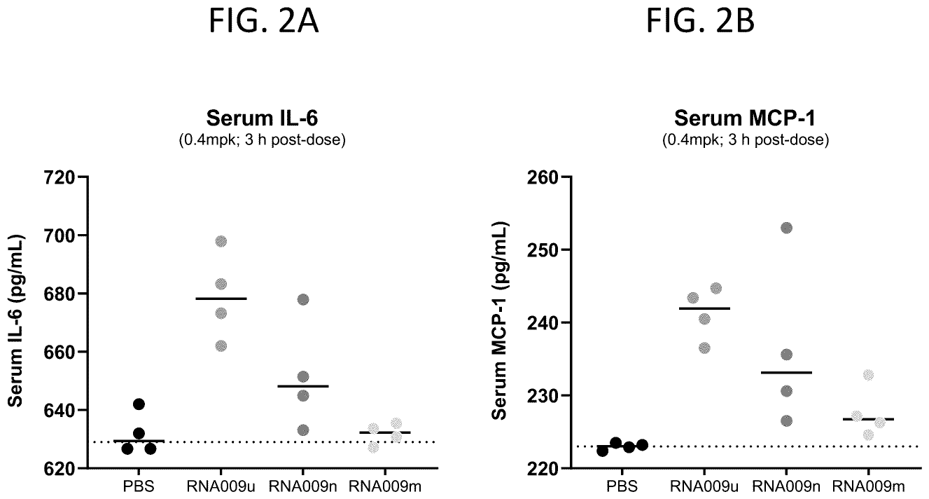

To evaluate tolerability, levels of IL-6 and MCP-1 inflammatory cytokines were measured in serum of mice in response to administration of co-formulated RNA-LNP. Briefly, mouse serum was isolated at 3 hours following administration of co-formulated RNA-LNP. MCP-1 and IL-6 were measured by MSD Mouse MCP-1 Kit and MSD Mouse IL-6 Kit (MESO SCALE DIAGNOSTICS, LLC (MSD)) according to the manufacturer's protocol. Both IL-6 (FIG. 2A) and MCP-1 (FIG. 2B) levels were elevated in mice that received RNA-009u when compared to control mice. However, cytokine levels were reduced for mice that received RNA-009 with uracil base modification (RNA-009n or RNA-009m).

Together, these data indicated RNA-009 incorporating an N1-methylpseudouridine modification provided highly efficient gene editing but did not induce aberrant innate immune activation.

### Example 3: Evaluation of Optimized mRNA for Editing the Albumin Locus in Mice

In vivo editing efficiency of sequence optimized SpCas9 RNA-009n was compared to SpCas9 parent mRNA in C57BL/6 mice. Parent mRNA and the RNA-009n transcripts were prepared as described in Example 1.

Editing efficiency was evaluated using gRNA targeting a sequence in the albumin gene locus of C57BL/6 mice (mAlbT1). The SpCas9 mRNA and mAlbT1 sgRNA were co-formulated as LNPs as described in Example 2.

C57BL/6 mice were administered co-formulated RNA-LNP containing RNA-009n or parent mRNA (N=4 per group). The mice were administered an intravenous injection by tail vein at a dose of 1.0 mpk. Liver tissue was isolated at approximately 96 hours following LNP administration. Editing efficiency was evaluated by TIDE analysis of liver tissue as described in Example 2. Editing efficiency of RNA-009n was evaluated for three independent mouse cohorts.

The frequency of INDEL formation at the albumin target sequence is shown in Table 7. The INDEL frequency exceeded 30% in each cohort treated with RNA-009n but was significantly lower (approximately 10%) for mice administered parent mRNA.

TABLE 7
In Vivo Editing Efficiency of Albumin Gene Locus in Mice

| mRNA | Frequency of INDELs (%) |
|---|---|
| Parent mRNA | 10.0 ± 0.6 |
| RNA-009n | 32.0 ± 3.6 |
| | 40.3 ± 6.6 |
| | 36.8 ± 1.2 |

### Example 4: Comparison of Optimized mRNA for Editing the Transferrin Gene Locus in Mice

In vivo editing efficiency and tolerability of RNA-009n was further compared to RNA-012 and RNA-013 with N1-methylpseudouridine modification (RNA-012n and RNA-013n respectively). The SpCas9 RNA-009n, RNA-012n, and RNA-013n transcripts were prepared as described in Example 1. SpCas9 mRNA and mTF_T2 sgRNA were co-formulated as LNPs as described in Example 2. Co-formulated RNA-LNP was administered by intravenous tail vein injection at a dose of 0.4 mpk. The RNA-LNP was diluted in PBS to enable administration in a volume of 10 mL per kg of body weight. Negative control mice received an intravenous injection of PBS alone.

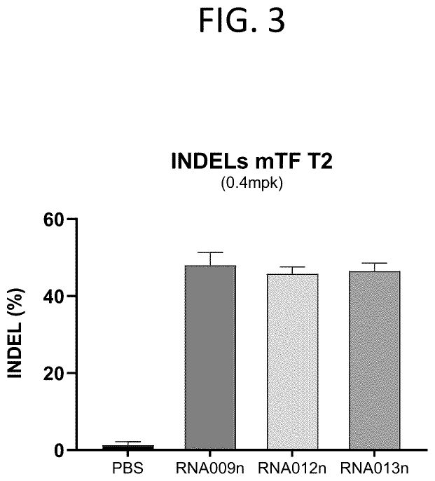

Editing efficiency was determined by measuring INDEL frequency at the transferrin target gene sequence in liver tissue as described in Example 2. Liver tissue was isolated at approximately 96 hours post-administration of co-formulated RNA-LNP. As shown in FIG. 3, the frequency of INDELs was similar for RNA-009n, RNA-012n, and RNA-013n.

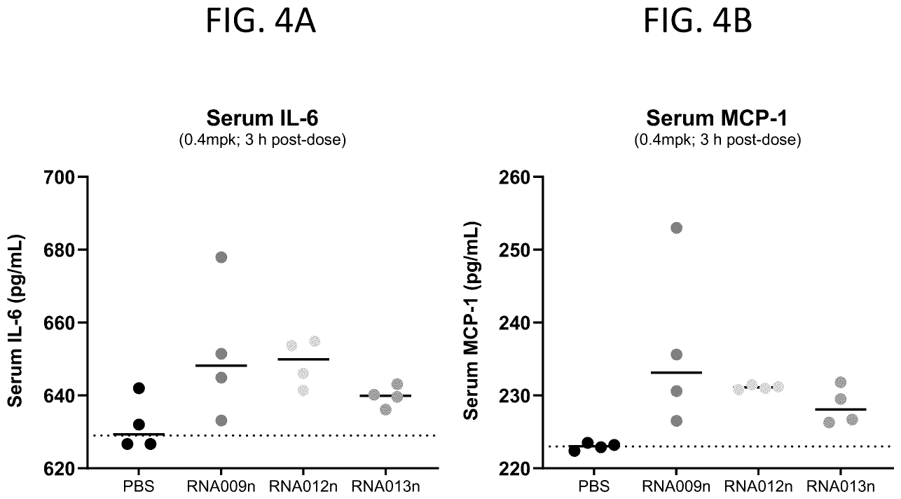

Additionally, tolerability was evaluated by measuring serum cytokine induction following administration of RNA-LNP as described in Example 2. Serum was collected at 3 hours post-administration of co-formulated RNA-LNP. As shown in FIGS. 4A-4B, both IL-6 and MCP-1 levels were comparable for each of the mRNAs evaluated.

These data indicated that the editing efficiency and tolerability of RNA-009n was not further improved with use of a uridine-depleted mRNA, such as that present in RNA-012n or RNA-013n.

### Example 5: Effect of mRNA Dose on Gene Editing in Mice

The effect of mRNA dose on editing efficiency of various gene targets was further evaluated for RNA-009n using sgRNA targeting the albumin, C3, or transferrin loci. The RNA-009n transcript was prepared, as described in Example 1, and co-formulated with the sgRNA as LNPs, as described in Example 2. Co-formulated mRNA-LNP was administered by intravenous tail vein injection at a dose of 1.0 mpk, 0.6 mpk, 0.4 mpk, 0.3 mpk, 0.25 mpk, or 0.2 mpk to C57BL/6 mice. Negative control mice received an intravenous injection of PBS alone.

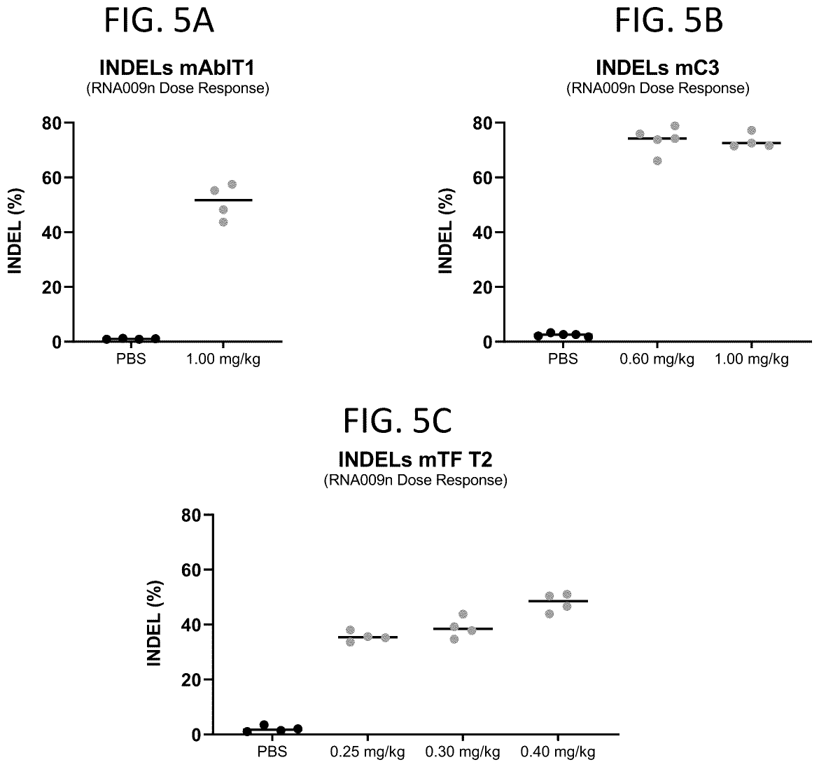

Editing efficiency was determined by measuring INDEL frequency at the albumin (FIG. 5A), C3 (FIG. 5B), and transferrin (FIG. 5C) loci in liver tissue, as described in Example 2. Liver tissue was isolated at approximately 96 hours post-administration of co-formulated RNA-LNP.

FIG. 5A shows editing efficiency for RNA-009n was high for the 1.0 mpk dose with the frequency of INDELs at the albumin target gene exceeding 40%. As shown in FIG. 5B, editing efficiency for RNA-009n was high for the 1.0 mpk dose and the 0.6 mpk dose, with the frequency of INDELs at the C3 target gene exceeding 70% at each dose. Furthermore, as shown in FIG. 5C, the frequency of INDELs at the transferrin target gene was above 35% for the 0.4 mpk, 0.3 mpk, and 0.25 mpk doses. Together, these data indicate efficient gene editing with RNA-009n.

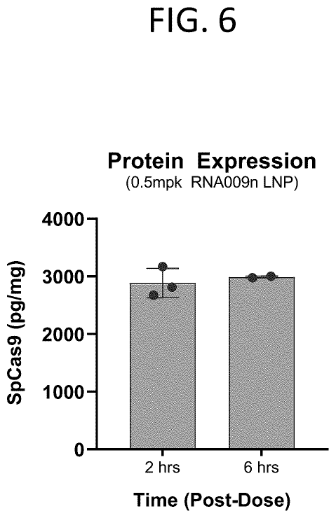

Furthermore, levels of Cas9 protein produced in liver tissue following administration of RNA-009n was evaluated. RNA-009n was formulated as an LNP preparation without gRNA. The RNA-LNP was administered by intravenous tail vein injection at a dose of 0.5 mg RNA-009n per kg of body weight to C57BL/6 mice. Liver tissue was isolated at 2 hours and 6 hours post administration. The liver tissue was homogenized and the quantity of SpCas9 present per mg of liver tissue was determined by electrochemiluminescence immunoassay. As shown in FIG. 6, SpCas9 levels exceeded 2000 pg/mg at each time point evaluated.

### Example 6: Evaluation of Optimized mRNA for Gene Editing in Non-human Primates

In vivo editing efficiency and tolerability of sequence optimized SpCas9 RNA-009 with N1-methylpseudouridine modification was further evaluated in non-human primate (NHPs). The SpCas9 RNA-009n transcripts were compared to unmodified RNA-012 (RNA-012u) or RNA-012 with N1-methylspeurouridine modification (RNA-012n). The RNA-009n, RNA-012u, and RNA-012n transcripts were prepared as described in Example 1.

The mRNA transcripts were evaluated for editing of a target gene sequence in the albumin gene locus. The SpCas9 mRNA and a sgRNA targeting the NHP albumin locus (hT5) were co-formulated as LNPs as described in Example 2. The NHPs were administered the co-formulated RNA-LNP by intravenous injection at a dose of 2 mg RNA (mRNA+gRNA) per kg of body weight (mg/kg).

To evaluate editing efficiency, liver tissue was isolated at 7 days post administration of RNA-LNP. Genomic DNA was extracted, the albumin target gene was amplified by PCR, and INDEL frequency was determined by TIDE analysis as described in Example 2.

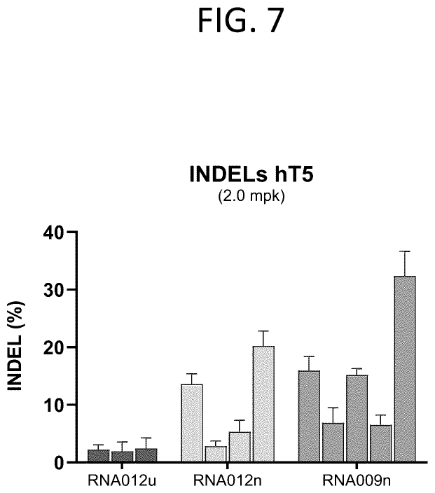

As shown in FIG. 7, administration of co-formulated RNA-LNP containing RNA-009n resulted in higher editing efficiency compared to RNA-LNP containing RNA-012u. The average INDEL frequency is shown in Table 8.

TABLE 8
In Vivo Editing Efficiency of Albumin Gene Locus in NHPs

| mRNA | # NHP per cohort | Average INDEL frequency (%) |
|---|---|---|
| RNA-012u | 3 | 2.2 ± 0.3 |
| RNA-012n | 4 | 10.5 ± 8.0 |
| RNA-009n | 5 | 15.4 ± 10.5 |

### SEQUENCE LISTING

| Name/ | | | |
|---|---|---|---|
| Description | Type | Sequence | SEQ ID NO |
| RNA-009 | DNA | AGAGGAAATAAGAGAGAAAAGAAGAGTAAGAAGAAATATAAGAGCCA | 1 |
| | | CCATGGCCCCTAAGAAGAAGAGAAAAGTCGGAATTCACGGAGTCCCC | |
| | | GCCGCCGACAAAAAGTACTCCATTGGCCTTGATATTGGAACCAACTC | |
| | | CGTGGGTTGGGCCGTGATCACTGACGAGTACAAGGTGCCGTCCAAGA | |
| | | AGTTCAAGGTGCTGGGGAACACTGACCGGCACTCAATTAAGAAGAAC | |
| | | CTGATTGGGGCGCTGCTGTTCGACTCCGGAGAAACCGCGGAGGCTAC | |
| | | CCGCCTGAAGCGGACTGCCCGGCGGAGATACACGCGCAGGAAGAACC | |
| | | GGATTTGCTACCTCCAAGAAATCTTCAGCAACGAAATGGCAAAGGTG | |
| | | GACGATTCCTTCTTCCATCGCCTGGAAGAGAGCTTCCTGGTGGAAGA | |
| | | GGACAAGAAGCACGAAAGACACCCGATTTTCGGCAACATCGTGGATG | |
| | | AGGTCGCATACCACGAAAAGTACCCCACCATCTATCATCTTCGGAAG | |
| | | AAGCTGGTCGACTCCACCGATAAGGCCGATCTGCGCCTGATCTACTT | |
| | | GGCGCTGGCTCACATGATTAAGTTCAGAGGACACTTTCTGATAGAGG | |
| | | GCGACCTCAATCCCGATAACTCCGACGTGGATAAGCTGTTCATCCAA | |
| | | CTGGTGCAGACGTACAACCAACTGTTTGAAGAGAATCCAATCAACGC | |
| | | CAGCGGGGTGGACGCCAAGGCCATCCTGTCCGCCCGGCTGTCAAAGT | |
| | | CCAGACGCCTGGAGAATCTCATCGCGCAACTCCCTGGCGAAAAAAAG | |
| | | AACGGACTCTTCGGGAATCTGATTGCTCTGTCCCTGGGGCTCACTCC | |
| | | GAACTTCAAGTCGAACTTCGACCTGGCGGAGGACGCTAAGCTGCAGC | |
| | | TGTCCAAGGACACCTACGATGACGATCTGGATAACCTTCTGGCCCAG | |
| | | ATCGGGGATCAATACGCCGATCTCTTCCTGGCCGCAAAGAACTTGTC | |
| | | GGATGCTATTCTGCTGAGCGACATTCTGCGGGTCAATACTGAAATCA | |
| | | CCAAGGCGCCCCTGTCGGCCAGCATGATCAAGCGCTACGACGAACAC | |
| | | CACCAAGACCTGACTCTGCTGAAGGCCCTCGTGCGCCAGCAGCTGCC | |
| | | TGAAAAGTACAAGGAGATTTTCTTCGACCAGTCCAAGAACGGATACG | |
| | | CCGGATACATTGACGGAGGGGCCAGCCAGGAGGAATTTTACAAATTC | |
| | | ATCAAGCCCATTCTCGAGAAAATGGACGGAACCGAAGAGTTGCTCGT | |
| | | GAAGCTGAACAGAGAGGATCTCCTCCGGAAGCAGCGGACCTTCGACA | |
| | | ACGGTTCCATCCCGCACCAAATCCACCTGGGCGAATTGCACGCCATC | |
| | | CTCCGGCGGCAGGAAGATTTCTACCCATTCTTGAAGGACAATCGCGA | |
| | | AAAGATCGAAAAGATCTTGACTTTCCGCATCCCGTACTACGTGGGCC | |
| | | CTCTGGCCCGCGGCAACTCCCGCTTCGCTTGGATGACACGGAAGTCC | |
| | | GAGGAAACCATTACGCCCTGGAACTTCGAGGAAGTGGTGGACAAGGG | |
| | | GGCGTCCGCCCAGAGCTTCATCGAACGCATGACCAATTTCGACAAGA | |
| | | ACCTCCCGAACGAAAAAGTGCTGCCAAAGCACTCGCTCCTCTACGAA | |
| | | TACTTCACCGTGTACAACGAGCTGACTAAGGTCAAATACGTGACTGA | |
| | | GGGAATGCGGAAGCCGGCCTTCCTGTCGGGAGAGCAGAAGAAGGCCA | |
| | | TAGTGGACTTGCTTTTCAAGACTAACCGGAAGGTCACTGTGAAGCAA | |
| | | CTCAAGGAGGACTACTTCAAGAAGATCGAGTGTTTCGACTCGGTGGA | |
| | | GATCTCGGGTGTCGAGGACCGCTTCAACGCCTCCCTGGGAACTTACC | |
| | | ACGATCTGCTGAAGATCATCAAGGACAAGGACTTCCTCGATAACGAA | |
| | | GAAAATGAGGACATCCTCGAGGATATCGTGCTGACCCTGACCTTGTT | |
| | | CGAGGATAGGGAGATGATCGAGGAGCGGCTCAAGACCTACGCCCACC | |
| | | TGTTTGACGACAAAGTGATGAAGCAACTGAAACGGCGGAGGTATACC | |
| | | GGCTGGGGTCGGCTGTCCCGCAAGCTGATCAACGGGATCAGGGACAA | |
| | | GCAGTCCGGAAAGACCATCCTCGACTTCCTTAAGTCCGACGGATTCG | |
| | | CGAACCGCAACTTCATGCAACTTATCCACGACGACTCGCTGACATTC | |
| | | AAGGAAGATATCCAGAAGGCCCAGGTGTCCGGACAGGGGGACTCGCT | |
| | | TCATGAGCACATCGCTAACCTGGCCGGATCCCCCGCCATAAAAAAGG | |
| | | GCATTCTGCAGACCGTCAAAGTGGTGGATGAGCTGGTCAAGGTCATG | |
| | | GGCCGGCATAAGCCGGAAAACATCGTCATCGAGATGGCCCGCGAGAA | |
| | | CCAGACTACGCAGAAGGGCCAGAAGAACTCCCGGGAGCGGATGAAGC | |
| | | GGATTGAAGAGGGCATCAAGGAGCTCGGCAGCCAGATTCTGAAGGAA | |
| | | CATCCCGTGGAAAACACCCAGCTGCAAAACGAAAAGCTCTATTTGTA | |
| | | CTATCTGCAAAACGGACGCGATATGTACGTGGATCAGGAGCTGGACA | |
| | | TTAACAGACTGAGCGACTATGACGTGGATCACATTGTGCCTCAAAGC | |
| | | TTCCTCAAGGACGACTCAATTGACAACAAGGTCCTGACCAGAAGCGA | |
| | | CAAGAACAGAGGAAAGTCGGATAATGTGCCGTCCGAAGAAGTGGTCA | |
| | | AGAAGATGAAGAATTACTGGAGACAGCTCCTGAATGCGAAGCTCATT | |
| | | ACCCAGCGGAAGTTCGATAACCTGACCAAGGCCGAAAGGGGTGGACT | |
| | | GTCCGAACTCGACAAAGCTGGCTTCATCAAGCGCCAACTGGTCGAAA | |
| | | CCAGGCAGATCACCAAGCACGTCGCCCAGATTCTGGACAGCCGCATG | |
| | | AACACTAAGTACGACGAGAACGATAAGCTGATCCGCGAAGTGAAGGT | |
| | | CATCACCCTGAAGTCCAAGCTCGTGTCCGACTTTCGGAAGGATTTCC | |
| | | AGTTTTACAAGGTCCGCGAGATCAACAACTACCATCACGCCCACGAC | |
| | | GCGTACCTTAACGCAGTCGTGGGAACGGCTCTTATCAAGAAGTACCC | |
| | | AAAGCTGGAGTCGGAATTTGTGTACGGAGACTACAAAGTGTACGACG | |
| | | TGCGCAAGATGATCGCCAAATCTGAGCAAGAGATCGGGAAGGCAACC | |
| | | GCCAAATACTTCTTCTACTCAAACATTATGAATTTTTTCAAAACTGA | |
| | | GATTACCCTGGCTAACGGAGAAATTCGGAAGCGCCCCCTGATTGAAA | |
| | | CCAACGGAGAAACTGGAGAAATTGTGTGGGACAAGGGACGGGACTTC | |
| | | GCCACCGTCCGCAAGGTCCTCTCAATGCCCCAAGTCAACATCGTGAA | |
| | | AAAGACCGAAGTGCAAACCGGCGGCTTCTCAAAGGAGTCCATCCTGC | |
| | | CTAAGCGCAACAGCGACAAGCTGATTGCCAGGAAGAAGGACTGGGAC | |
| | | CCGAAGAAGTACGGAGGATTTGATTCCCCTACCGTGGCCTACTCCGT | |
| | | GCTCGTGGTGGCCAAAGTGGAAAAGGGGAAATCCAAGAAGCTGAAGT | |
| | | CGGTGAAGGAGCTTTTGGGTATCACCATCATGGAACGCTCCTCGTTC | |
| | | GAAAAGAACCCAATCGATTTCCTGGAAGCTAAGGGTTATAAGGAAGT | |
| | | GAAAAAGGACCTGATTATCAAGCTGCCCAAGTACTCACTGTTCGAGC | |
| | | TGGAAAACGGTCGGAAAAGGATGCTGGCCAGCGCCGGAGAACTCCAG | |
| | | AAGGGAAACGAACTGGCACTGCCGTCCAAATACGTCAACTTCCTCTA | |
| | | CCTTGCATCCCATTACGAAAAACTCAAGGGATCGCCGGAGGACAACG | |
| | | AGCAGAAGCAGCTTTTCGTGGAGCAACACAAGCATTACTTGGACGAG | |
| | | ATCATCGAGCAGATTTCCGAGTTCTCAAAGCGCGTGATCCTGGCCGA | |
| | | CGCAAATCTGGACAAGGTCCTGTCCGCGTACAATAAGCATCGGGACA | |
| | | AGCCTATCCGCGAACAGGCCGAGAACATCATCCATCTGTTCACTCTG | |
| | | ACAAACCTGGGCGCACCCGCCGCGTTCAAGTACTTTGACACCACCAT | |
| | | CGATAGGAAGCGATACACCTCAACTAAGGAAGTGTTGGACGCGACCC | |
| | | TTATCCATCAGTCGATCACCGGGCTGTACGAAACACGGATCGACCTC | |
| | | AGCCAGTTGGGAGGCGACAAGCGCCCTGCGGCTACCAAGAAGGCCGG | |
| | | ACAGGCCAAGAAGAAGAAATGAGCGGCCGCTTAATTAAGCTGCCTTC | |
| | | TGCGGGGCTTGCCTTCTGGCCATGCCCTTCTTCTCTCCCTTGCACCT | |
| | | GTACCTCTTGGTCTTTGAATAAAGCCTGAGTAGGAAGTCTAGAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAA | |
| RNA-009 | RNA | AGAGGAAAUAAGAGAGAAAAGAAGAGUAAGAAGAAAUAUAAGAGCCA | 2 |
| Full-length | | CCAUGGCCCCUAAGAAGAAGAGAAAAGUCGGAAUUCACGGAGUCCCC | |
| mRNA | | GCCGCCGACAAAAAGUACUCCAUUGGCCUUGAUAUUGGAACCAACUC | |
| 5′ UTR IN | | CGUGGGUUGGGCCGUGAUCACUGACGAGUACAAGGUGCCGUCCAAGA | |
| BOLD | | AGUUCAAGGUGCUGGGGAACACUGACCGGCACUCAAUUAAGAAGAAC | |
| 3′ UTR IN | | CUGAUUGGGGCGCUGCUGUUCGACUCCGGAGAAACCGCGGAGGCUAC | |
| BOLD ITALICS | | CCGCCUGAAGCGGACUGCCCGGCGGAGAUACACGCGCAGGAAGAACC | |
| POLY-A TAIL | | GGAUUUGCUACCUCCAAGAAAUCUUCAGCAACGAAAUGGCAAAGGUG | |
| IN UNDERLINE | | GACGAUUCCUUCUUCCAUCGCCUGGAAGAGAGCUUCCUGGUGGAAGA | |
| | | GGACAAGAAGCACGAAAGACACCCGAUUUUCGGCAACAUCGUGGAUG | |
| | | AGGUCGCAUACCACGAAAAGUACCCCACCAUCUAUCAUCUUCGGAAG | |
| | | AAGCUGGUCGACUCCACCGAUAAGGCCGAUCUGCGCCUGAUCUACUU | |
| | | GGCGCUGGCUCACAUGAUUAAGUUCAGAGGACACUUUCUGAUAGAGG | |
| | | GCGACCUCAAUCCCGAUAACUCCGACGUGGAUAAGCUGUUCAUCCAA | |
| | | CUGGUGCAGACGUACAACCAACUGUUUGAAGAGAAUCCAAUCAACGC | |
| | | CAGCGGGGUGGACGCCAAGGCCAUCCUGUCCGCCCGGCUGUCAAAGU | |
| | | CCAGACGCCUGGAGAAUCUCAUCGCGCAACUCCCUGGCGAAAAAAAG | |
| | | AACGGACUCUUCGGGAAUCUGAUUGCUCUGUCCCUGGGGCUCACUCC | |
| | | GAACUUCAAGUCGAACUUCGACCUGGCGGAGGACGCUAAGCUGCAGC | |
| | | UGUCCAAGGACACCUACGAUGACGAUCUGGAUAACCUUCUGGCCCAG | |
| | | AUCGGGGAUCAAUACGCCGAUCUCUUCCUGGCCGCAAAGAACUUGUC | |
| | | GGAUGCUAUUCUGCUGAGCGACAUUCUGCGGGUCAAUACUGAAAUCA | |
| | | CCAAGGCGCCCCUGUCGGCCAGCAUGAUCAAGCGCUACGACGAACAC | |
| | | CACCAAGACCUGACUCUGCUGAAGGCCCUCGUGCGCCAGCAGCUGCC | |
| | | UGAAAAGUACAAGGAGAUUUUCUUCGACCAGUCCAAGAACGGAUACG | |
| | | CCGGAUACAUUGACGGAGGGGCCAGCCAGGAGGAAUUUUACAAAUUC | |
| | | AUCAAGCCCAUUCUCGAGAAAAUGGACGGAACCGAAGAGUUGCUCGU | |
| | | GAAGCUGAACAGAGAGGAUCUCCUCCGGAAGCAGCGGACCUUCGACA | |
| | | ACGGUUCCAUCCCGCACCAAAUCCACCUGGGCGAAUUGCACGCCAUC | |
| | | CUCCGGCGGCAGGAAGAUUUCUACCCAUUCUUGAAGGACAAUCGCGA | |
| | | AAAGAUCGAAAAGAUCUUGACUUUCCGCAUCCCGUACUACGUGGGCC | |
| | | CUCUGGCCCGCGGCAACUCCCGCUUCGCUUGGAUGACACGGAAGUCC | |
| | | GAGGAAACCAUUACGCCCUGGAACUUCGAGGAAGUGGUGGACAAGGG | |
| | | GGCGUCCGCCCAGAGCUUCAUCGAACGCAUGACCAAUUUCGACAAGA | |
| | | ACCUCCCGAACGAAAAAGUGCUGCCAAAGCACUCGCUCCUCUACGAA | |
| | | UACUUCACCGUGUACAACGAGCUGACUAAGGUCAAAUACGUGACUGA | |
| | | GGGAAUGCGGAAGCCGGCCUUCCUGUCGGGAGAGCAGAAGAAGGCCA | |
| | | UAGUGGACUUGCUUUUCAAGACUAACCGGAAGGUCACUGUGAAGCAA | |
| | | CUCAAGGAGGACUACUUCAAGAAGAUCGAGUGUUUCGACUCGGUGGA | |
| | | GAUCUCGGGUGUCGAGGACCGCUUCAACGCCUCCCUGGGAACUUACC | |
| | | ACGAUCUGCUGAAGAUCAUCAAGGACAAGGACUUCCUCGAUAACGAA | |
| | | GAAAAUGAGGACAUCCUCGAGGAUAUCGUGCUGACCCUGACCUUGUU | |
| | | CGAGGAUAGGGAGAUGAUCGAGGAGCGGCUCAAGACCUACGCCCACC | |
| | | UGUUUGACGACAAAGUGAUGAAGCAACUGAAACGGCGGAGGUAUACC | |
| | | GGCUGGGGUCGGCUGUCCCGCAAGCUGAUCAACGGGAUCAGGGACAA | |
| | | GCAGUCCGGAAAGACCAUCCUCGACUUCCUUAAGUCCGACGGAUUCG | |
| | | CGAACCGCAACUUCAUGCAACUUAUCCACGACGACUCGCUGACAUUC | |
| | | AAGGAAGAUAUCCAGAAGGCCCAGGUGUCCGGACAGGGGGACUCGCU | |
| | | UCAUGAGCACAUCGCUAACCUGGCCGGAUCCCCCGCCAUAAAAAAGG | |
| | | GCAUUCUGCAGACCGUCAAAGUGGUGGAUGAGCUGGUCAAGGUCAUG | |
| | | GGCCGGCAUAAGCCGGAAAACAUCGUCAUCGAGAUGGCCCGCGAGAA | |
| | | CCAGACUACGCAGAAGGGCCAGAAGAACUCCCGGGAGCGGAUGAAGC | |
| | | GGAUUGAAGAGGGCAUCAAGGAGCUCGGCAGCCAGAUUCUGAAGGAA | |
| | | CAUCCCGUGGAAAACACCCAGCUGCAAAACGAAAAGCUCUAUUUGUA | |
| | | CUAUCUGCAAAACGGACGCGAUAUGUACGUGGAUCAGGAGCUGGACA | |
| | | UUAACAGACUGAGCGACUAUGACGUGGAUCACAUUGUGCCUCAAAGC | |
| | | UUCCUCAAGGACGACUCAAUUGACAACAAGGUCCUGACCAGAAGCGA | |
| | | CAAGAACAGAGGAAAGUCGGAUAAUGUGCCGUCCGAAGAAGUGGUCA | |
| | | AGAAGAUGAAGAAUUACUGGAGACAGCUCCUGAAUGCGAAGCUCAUU | |
| | | ACCCAGCGGAAGUUCGAUAACCUGACCAAGGCCGAAAGGGGUGGACU | |
| | | GUCCGAACUCGACAAAGCUGGCUUCAUCAAGCGCCAACUGGUCGAAA | |
| | | CCAGGCAGAUCACCAAGCACGUCGCCCAGAUUCUGGACAGCCGCAUG | |
| | | AACACUAAGUACGACGAGAACGAUAAGCUGAUCCGCGAAGUGAAGGU | |
| | | CAUCACCCUGAAGUCCAAGCUCGUGUCCGACUUUCGGAAGGAUUUCC | |
| | | AGUUUUACAAGGUCCGCGAGAUCAACAACUACCAUCACGCCCACGAC | |
| | | GCGUACCUUAACGCAGUCGUGGGAACGGCUCUUAUCAAGAAGUACCC | |
| | | AAAGCUGGAGUCGGAAUUUGUGUACGGAGACUACAAAGUGUACGACG | |
| | | UGCGCAAGAUGAUCGCCAAAUCUGAGCAAGAGAUCGGGAAGGCAACC | |
| | | GCCAAAUACUUCUUCUACUCAAACAUUAUGAAUUUUUUCAAAACUGA | |
| | | GAUUACCCUGGCUAACGGAGAAAUUCGGAAGCGCCCCCUGAUUGAAA | |
| | | CCAACGGAGAAACUGGAGAAAUUGUGUGGGACAAGGGACGGGACUUC | |
| | | GCCACCGUCCGCAAGGUCCUCUCAAUGCCCCAAGUCAACAUCGUGAA | |
| | | AAAGACCGAAGUGCAAACCGGCGGCUUCUCAAAGGAGUCCAUCCUGC | |
| | | CUAAGCGCAACAGCGACAAGCUGAUUGCCAGGAAGAAGGACUGGGAC | |
| | | CCGAAGAAGUACGGAGGAUUUGAUUCCCCUACCGUGGCCUACUCCGU | |
| | | GCUCGUGGUGGCCAAAGUGGAAAAGGGGAAAUCCAAGAAGCUGAAGU | |
| | | CGGUGAAGGAGCUUUUGGGUAUCACCAUCAUGGAACGCUCCUCGUUC | |
| | | GAAAAGAACCCAAUCGAUUUCCUGGAAGCUAAGGGUUAUAAGGAAGU | |
| | | GAAAAAGGACCUGAUUAUCAAGCUGCCCAAGUACUCACUGUUCGAGC | |
| | | UGGAAAACGGUCGGAAAAGGAUGCUGGCCAGCGCCGGAGAACUCCAG | |
| | | AAGGGAAACGAACUGGCACUGCCGUCCAAAUACGUCAACUUCCUCUA | |
| | | CCUUGCAUCCCAUUACGAAAAACUCAAGGGAUCGCCGGAGGACAACG | |
| | | AGCAGAAGCAGCUUUUCGUGGAGCAACACAAGCAUUACUUGGACGAG | |
| | | AUCAUCGAGCAGAUUUCCGAGUUCUCAAAGCGCGUGAUCCUGGCCGA | |
| | | CGCAAAUCUGGACAAGGUCCUGUCCGCGUACAAUAAGCAUCGGGACA | |
| | | AGCCUAUCCGCGAACAGGCCGAGAACAUCAUCCAUCUGUUCACUCUG | |
| | | ACAAACCUGGGCGCACCCGCCGCGUUCAAGUACUUUGACACCACCAU | |
| | | CGAUAGGAAGCGAUACACCUCAACUAAGGAAGUGUUGGACGCGACCC | |
| | | UUAUCCAUCAGUCGAUCACCGGGCUGUACGAAACACGGAUCGACCUC | |
| | | AGCCAGUUGGGAGGCGACAAGCGCCCUGCGGCUACCAAGAAGGCCGG | |
| | | ACAGGCCAAGAAGAAGAAAUGAGCGGCCGCUUAAUUAAGCUGCCUUC | |
| | | UGCGGGGCUUGCCUUCUGGCCAUGCCCUUCUUCUCUCCCUUGCACCU | |
| | | GUACCUCUUGGUCUUUGAAUAAAGCCUGAGUAGGAAGUCUAGAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAA | |
| RNA-009 | DNA | ATGGCCCCTAAGAAGAAGAGAAAAGTCGGAATTCACGGAGTCCCCGC | 3 |
| coding region | | CGCCGACAAAAAGTACTCCATTGGCCTTGATATTGGAACCAACTCCG | |
| | | TGGGTTGGGCCGTGATCACTGACGAGTACAAGGTGCCGTCCAAGAAG | |
| | | TTCAAGGTGCTGGGGAACACTGACCGGCACTCAATTAAGAAGAACCT | |
| | | GATTGGGGCGCTGCTGTTCGACTCCGGAGAAACCGCGGAGGCTACCC | |
| | | GCCTGAAGCGGACTGCCCGGCGGAGATACACGCGCAGGAAGAACCGG | |
| | | ATTTGCTACCTCCAAGAAATCTTCAGCAACGAAATGGCAAAGGTGGA | |
| | | CGATTCCTTCTTCCATCGCCTGGAAGAGAGCTTCCTGGTGGAAGAGG | |
| | | ACAAGAAGCACGAAAGACACCCGATTTTCGGCAACATCGTGGATGAG | |
| | | GTCGCATACCACGAAAAGTACCCCACCATCTATCATCTTCGGAAGAA | |
| | | GCTGGTCGACTCCACCGATAAGGCCGATCTGCGCCTGATCTACTTGG | |
| | | CGCTGGCTCACATGATTAAGTTCAGAGGACACTTTCTGATAGAGGGC | |
| | | GACCTCAATCCCGATAACTCCGACGTGGATAAGCTGTTCATCCAACT | |
| | | GGTGCAGACGTACAACCAACTGTTTGAAGAGAATCCAATCAACGCCA | |
| | | GCGGGGTGGACGCCAAGGCCATCCTGTCCGCCCGGCTGTCAAAGTCC | |
| | | AGACGCCTGGAGAATCTCATCGCGCAACTCCCTGGCGAAAAAAAGAA | |
| | | CGGACTCTTCGGGAATCTGATTGCTCTGTCCCTGGGGCTCACTCCGA | |
| | | ACTTCAAGTCGAACTTCGACCTGGCGGAGGACGCTAAGCTGCAGCTG | |
| | | TCCAAGGACACCTACGATGACGATCTGGATAACCTTCTGGCCCAGAT | |
| | | CGGGGATCAATACGCCGATCTCTTCCTGGCCGCAAAGAACTTGTCGG | |
| | | ATGCTATTCTGCTGAGCGACATTCTGCGGGTCAATACTGAAATCACC | |
| | | AAGGCGCCCCTGTCGGCCAGCATGATCAAGCGCTACGACGAACACCA | |
| | | CCAAGACCTGACTCTGCTGAAGGCCCTCGTGCGCCAGCAGCTGCCTG | |
| | | AAAAGTACAAGGAGATTTTCTTCGACCAGTCCAAGAACGGATACGCC | |
| | | GGATACATTGACGGAGGGGCCAGCCAGGAGGAATTTTACAAATTCAT | |
| | | CAAGCCCATTCTCGAGAAAATGGACGGAACCGAAGAGTTGCTCGTGA | |
| | | AGCTGAACAGAGAGGATCTCCTCCGGAAGCAGCGGACCTTCGACAAC | |
| | | GGTTCCATCCCGCACCAAATCCACCTGGGCGAATTGCACGCCATCCT | |
| | | CCGGCGGCAGGAAGATTTCTACCCATTCTTGAAGGACAATCGCGAAA | |
| | | AGATCGAAAAGATCTTGACTTTCCGCATCCCGTACTACGTGGGCCCT | |
| | | CTGGCCCGCGGCAACTCCCGCTTCGCTTGGATGACACGGAAGTCCGA | |
| | | GGAAACCATTACGCCCTGGAACTTCGAGGAAGTGGTGGACAAGGGGG | |
| | | CGTCCGCCCAGAGCTTCATCGAACGCATGACCAATTTCGACAAGAAC | |
| | | CTCCCGAACGAAAAAGTGCTGCCAAAGCACTCGCTCCTCTACGAATA | |
| | | CTTCACCGTGTACAACGAGCTGACTAAGGTCAAATACGTGACTGAGG | |
| | | GAATGCGGAAGCCGGCCTTCCTGTCGGGAGAGCAGAAGAAGGCCATA | |
| | | GTGGACTTGCTTTTCAAGACTAACCGGAAGGTCACTGTGAAGCAACT | |
| | | CAAGGAGGACTACTTCAAGAAGATCGAGTGTTTCGACTCGGTGGAGA | |
| | | TCTCGGGTGTCGAGGACCGCTTCAACGCCTCCCTGGGAACTTACCAC | |
| | | GATCTGCTGAAGATCATCAAGGACAAGGACTTCCTCGATAACGAAGA | |
| | | AAATGAGGACATCCTCGAGGATATCGTGCTGACCCTGACCTTGTTCG | |
| | | AGGATAGGGAGATGATCGAGGAGCGGCTCAAGACCTACGCCCACCTG | |
| | | TTTGACGACAAAGTGATGAAGCAACTGAAACGGCGGAGGTATACCGG | |
| | | CTGGGGTCGGCTGTCCCGCAAGCTGATCAACGGGATCAGGGACAAGC | |
| | | AGTCCGGAAAGACCATCCTCGACTTCCTTAAGTCCGACGGATTCGCG | |
| | | AACCGCAACTTCATGCAACTTATCCACGACGACTCGCTGACATTCAA | |
| | | GGAAGATATCCAGAAGGCCCAGGTGTCCGGACAGGGGGACTCGCTTC | |
| | | ATGAGCACATCGCTAACCTGGCCGGATCCCCCGCCATAAAAAAGGGC | |
| | | ATTCTGCAGACCGTCAAAGTGGTGGATGAGCTGGTCAAGGTCATGGG | |
| | | CCGGCATAAGCCGGAAAACATCGTCATCGAGATGGCCCGCGAGAACC | |
| | | AGACTACGCAGAAGGGCCAGAAGAACTCCCGGGAGCGGATGAAGCGG | |
| | | ATTGAAGAGGGCATCAAGGAGCTCGGCAGCCAGATTCTGAAGGAACA | |
| | | TCCCGTGGAAAACACCCAGCTGCAAAACGAAAAGCTCTATTTGTACT | |
| | | ATCTGCAAAACGGACGCGATATGTACGTGGATCAGGAGCTGGACATT | |
| | | AACAGACTGAGCGACTATGACGTGGATCACATTGTGCCTCAAAGCTT | |
| | | CCTCAAGGACGACTCAATTGACAACAAGGTCCTGACCAGAAGCGACA | |
| | | AGAACAGAGGAAAGTCGGATAATGTGCCGTCCGAAGAAGTGGTCAAG | |
| | | AAGATGAAGAATTACTGGAGACAGCTCCTGAATGCGAAGCTCATTAC | |
| | | CCAGCGGAAGTTCGATAACCTGACCAAGGCCGAAAGGGGTGGACTGT | |
| | | CCGAACTCGACAAAGCTGGCTTCATCAAGCGCCAACTGGTCGAAACC | |
| | | AGGCAGATCACCAAGCACGTCGCCCAGATTCTGGACAGCCGCATGAA | |
| | | CACTAAGTACGACGAGAACGATAAGCTGATCCGCGAAGTGAAGGTCA | |
| | | TCACCCTGAAGTCCAAGCTCGTGTCCGACTTTCGGAAGGATTTCCAG | |
| | | TTTTACAAGGTCCGCGAGATCAACAACTACCATCACGCCCACGACGC | |
| | | GTACCTTAACGCAGTCGTGGGAACGGCTCTTATCAAGAAGTACCCAA | |
| | | AGCTGGAGTCGGAATTTGTGTACGGAGACTACAAAGTGTACGACGTG | |
| | | CGCAAGATGATCGCCAAATCTGAGCAAGAGATCGGGAAGGCAACCGC | |
| | | CAAATACTTCTTCTACTCAAACATTATGAATTTTTTCAAAACTGAGA | |
| | | TTACCCTGGCTAACGGAGAAATTCGGAAGCGCCCCCTGATTGAAACC | |
| | | AACGGAGAAACTGGAGAAATTGTGTGGGACAAGGGACGGGACTTCGC | |
| | | CACCGTCCGCAAGGTCCTCTCAATGCCCCAAGTCAACATCGTGAAAA | |
| | | AGACCGAAGTGCAAACCGGCGGCTTCTCAAAGGAGTCCATCCTGCCT | |
| | | AAGCGCAACAGCGACAAGCTGATTGCCAGGAAGAAGGACTGGGACCC | |
| | | GAAGAAGTACGGAGGATTTGATTCCCCTACCGTGGCCTACTCCGTGC | |
| | | TCGTGGTGGCCAAAGTGGAAAAGGGGAAATCCAAGAAGCTGAAGTCG | |
| | | GTGAAGGAGCTTTTGGGTATCACCATCATGGAACGCTCCTCGTTCGA | |
| | | AAAGAACCCAATCGATTTCCTGGAAGCTAAGGGTTATAAGGAAGTGA | |
| | | AAAAGGACCTGATTATCAAGCTGCCCAAGTACTCACTGTTCGAGCTG | |
| | | GAAAACGGTCGGAAAAGGATGCTGGCCAGCGCCGGAGAACTCCAGAA | |
| | | GGGAAACGAACTGGCACTGCCGTCCAAATACGTCAACTTCCTCTACC | |
| | | TTGCATCCCATTACGAAAAACTCAAGGGATCGCCGGAGGACAACGAG | |
| | | CAGAAGCAGCTTTTCGTGGAGCAACACAAGCATTACTTGGACGAGAT | |
| | | CATCGAGCAGATTTCCGAGTTCTCAAAGCGCGTGATCCTGGCCGACG | |
| | | CAAATCTGGACAAGGTCCTGTCCGCGTACAATAAGCATCGGGACAAG | |
| | | CCTATCCGCGAACAGGCCGAGAACATCATCCATCTGTTCACTCTGAC | |
| | | AAACCTGGGCGCACCCGCCGCGTTCAAGTACTTTGACACCACCATCG | |
| | | ATAGGAAGCGATACACCTCAACTAAGGAAGTGTTGGACGCGACCCTT | |
| | | ATCCATCAGTCGATCACCGGGCTGTACGAAACACGGATCGACCTCAG | |
| | | CCAGTTGGGAGGCGACAAGCGCCCTGCGGCTACCAAGAAGGCCGGAC | |
| | | AGGCCAAGAAGAAGAAATGA | |
| RNA-009 | RNA | AUGGCCCCUAAGAAGAAGAGAAAAGUCGGAAUUCACGGAGUCCCCGC | 4 |
| coding region | | CGCCGACAAAAAGUACUCCAUUGGCCUUGAUAUUGGAACCAACUCCG | |
| | | UGGGUUGGGCCGUGAUCACUGACGAGUACAAGGUGCCGUCCAAGAAG | |
| | | UUCAAGGUGCUGGGGAACACUGACCGGCACUCAAUUAAGAAGAACCU | |
| | | GAUUGGGGCGCUGCUGUUCGACUCCGGAGAAACCGCGGAGGCUACCC | |
| | | GCCUGAAGCGGACUGCCCGGCGGAGAUACACGCGCAGGAAGAACCGG | |
| | | AUUUGCUACCUCCAAGAAAUCUUCAGCAACGAAAUGGCAAAGGUGGA | |
| | | CGAUUCCUUCUUCCAUCGCCUGGAAGAGAGCUUCCUGGUGGAAGAGG | |
| | | ACAAGAAGCACGAAAGACACCCGAUUUUCGGCAACAUCGUGGAUGAG | |
| | | GUCGCAUACCACGAAAAGUACCCCACCAUCUAUCAUCUUCGGAAGAA | |
| | | GCUGGUCGACUCCACCGAUAAGGCCGAUCUGCGCCUGAUCUACUUGG | |
| | | CGCUGGCUCACAUGAUUAAGUUCAGAGGACACUUUCUGAUAGAGGGC | |
| | | GACCUCAAUCCCGAUAACUCCGACGUGGAUAAGCUGUUCAUCCAACU | |
| | | GGUGCAGACGUACAACCAACUGUUUGAAGAGAAUCCAAUCAACGCCA | |
| | | GCGGGGUGGACGCCAAGGCCAUCCUGUCCGCCCGGCUGUCAAAGUCC | |
| | | AGACGCCUGGAGAAUCUCAUCGCGCAACUCCCUGGCGAAAAAAAGAA | |
| | | CGGACUCUUCGGGAAUCUGAUUGCUCUGUCCCUGGGGCUCACUCCGA | |
| | | ACUUCAAGUCGAACUUCGACCUGGCGGAGGACGCUAAGCUGCAGCUG | |
| | | UCCAAGGACACCUACGAUGACGAUCUGGAUAACCUUCUGGCCCAGAU | |
| | | CGGGGAUCAAUACGCCGAUCUCUUCCUGGCCGCAAAGAACUUGUCGG | |
| | | AUGCUAUUCUGCUGAGCGACAUUCUGCGGGUCAAUACUGAAAUCACC | |
| | | AAGGCGCCCCUGUCGGCCAGCAUGAUCAAGCGCUACGACGAACACCA | |
| | | CCAAGACCUGACUCUGCUGAAGGCCCUCGUGCGCCAGCAGCUGCCUG | |
| | | AAAAGUACAAGGAGAUUUUCUUCGACCAGUCCAAGAACGGAUACGCC | |
| | | GGAUACAUUGACGGAGGGGCCAGCCAGGAGGAAUUUUACAAAUUCAU | |
| | | CAAGCCCAUUCUCGAGAAAAUGGACGGAACCGAAGAGUUGCUCGUGA | |
| | | AGCUGAACAGAGAGGAUCUCCUCCGGAAGCAGCGGACCUUCGACAAC | |
| | | GGUUCCAUCCCGCACCAAAUCCACCUGGGCGAAUUGCACGCCAUCCU | |
| | | CCGGCGGCAGGAAGAUUUCUACCCAUUCUUGAAGGACAAUCGCGAAA | |
| | | AGAUCGAAAAGAUCUUGACUUUCCGCAUCCCGUACUACGUGGGCCCU | |
| | | CUGGCCCGCGGCAACUCCCGCUUCGCUUGGAUGACACGGAAGUCCGA | |
| | | GGAAACCAUUACGCCCUGGAACUUCGAGGAAGUGGUGGACAAGGGGG | |
| | | CGUCCGCCCAGAGCUUCAUCGAACGCAUGACCAAUUUCGACAAGAAC | |
| | | CUCCCGAACGAAAAAGUGCUGCCAAAGCACUCGCUCCUCUACGAAUA | |
| | | CUUCACCGUGUACAACGAGCUGACUAAGGUCAAAUACGUGACUGAGG | |
| | | GAAUGCGGAAGCCGGCCUUCCUGUCGGGAGAGCAGAAGAAGGCCAUA | |
| | | GUGGACUUGCUUUUCAAGACUAACCGGAAGGUCACUGUGAAGCAACU | |
| | | CAAGGAGGACUACUUCAAGAAGAUCGAGUGUUUCGACUCGGUGGAGA | |
| | | UCUCGGGUGUCGAGGACCGCUUCAACGCCUCCCUGGGAACUUACCAC | |
| | | GAUCUGCUGAAGAUCAUCAAGGACAAGGACUUCCUCGAUAACGAAGA | |
| | | AAAUGAGGACAUCCUCGAGGAUAUCGUGCUGACCCUGACCUUGUUCG | |
| | | AGGAUAGGGAGAUGAUCGAGGAGCGGCUCAAGACCUACGCCCACCUG | |
| | | UUUGACGACAAAGUGAUGAAGCAACUGAAACGGCGGAGGUAUACCGG | |
| | | CUGGGGUCGGCUGUCCCGCAAGCUGAUCAACGGGAUCAGGGACAAGC | |
| | | AGUCCGGAAAGACCAUCCUCGACUUCCUUAAGUCCGACGGAUUCGCG | |
| | | AACCGCAACUUCAUGCAACUUAUCCACGACGACUCGCUGACAUUCAA | |
| | | GGAAGAUAUCCAGAAGGCCCAGGUGUCCGGACAGGGGGACUCGCUUC | |
| | | AUGAGCACAUCGCUAACCUGGCCGGAUCCCCCGCCAUAAAAAAGGGC | |
| | | AUUCUGCAGACCGUCAAAGUGGUGGAUGAGCUGGUCAAGGUCAUGGG | |
| | | CCGGCAUAAGCCGGAAAACAUCGUCAUCGAGAUGGCCCGCGAGAACC | |
| | | AGACUACGCAGAAGGGCCAGAAGAACUCCCGGGAGCGGAUGAAGCGG | |
| | | AUUGAAGAGGGCAUCAAGGAGCUCGGCAGCCAGAUUCUGAAGGAACA | |
| | | UCCCGUGGAAAACACCCAGCUGCAAAACGAAAAGCUCUAUUUGUACU | |
| | | AUCUGCAAAACGGACGCGAUAUGUACGUGGAUCAGGAGCUGGACAUU | |
| | | AACAGACUGAGCGACUAUGACGUGGAUCACAUUGUGCCUCAAAGCUU | |
| | | CCUCAAGGACGACUCAAUUGACAACAAGGUCCUGACCAGAAGCGACA | |
| | | AGAACAGAGGAAAGUCGGAUAAUGUGCCGUCCGAAGAAGUGGUCAAG | |
| | | AAGAUGAAGAAUUACUGGAGACAGCUCCUGAAUGCGAAGCUCAUUAC | |
| | | CCAGCGGAAGUUCGAUAACCUGACCAAGGCCGAAAGGGGUGGACUGU | |
| | | CCGAACUCGACAAAGCUGGCUUCAUCAAGCGCCAACUGGUCGAAACC | |
| | | AGGCAGAUCACCAAGCACGUCGCCCAGAUUCUGGACAGCCGCAUGAA | |
| | | CACUAAGUACGACGAGAACGAUAAGCUGAUCCGCGAAGUGAAGGUCA | |
| | | UCACCCUGAAGUCCAAGCUCGUGUCCGACUUUCGGAAGGAUUUCCAG | |
| | | UUUUACAAGGUCCGCGAGAUCAACAACUACCAUCACGCCCACGACGC | |
| | | GUACCUUAACGCAGUCGUGGGAACGGCUCUUAUCAAGAAGUACCCAA | |
| | | AGCUGGAGUCGGAAUUUGUGUACGGAGACUACAAAGUGUACGACGUG | |
| | | CGCAAGAUGAUCGCCAAAUCUGAGCAAGAGAUCGGGAAGGCAACCGC | |
| | | CAAAUACUUCUUCUACUCAAACAUUAUGAAUUUUUUCAAAACUGAGA | |
| | | UUACCCUGGCUAACGGAGAAAUUCGGAAGCGCCCCCUGAUUGAAACC | |
| | | AACGGAGAAACUGGAGAAAUUGUGUGGGACAAGGGACGGGACUUCGC | |
| | | CACCGUCCGCAAGGUCCUCUCAAUGCCCCAAGUCAACAUCGUGAAAA | |
| | | AGACCGAAGUGCAAACCGGCGGCUUCUCAAAGGAGUCCAUCCUGCCU | |
| | | AAGCGCAACAGCGACAAGCUGAUUGCCAGGAAGAAGGACUGGGACCC | |
| | | GAAGAAGUACGGAGGAUUUGAUUCCCCUACCGUGGCCUACUCCGUGC | |
| | | UCGUGGUGGCCAAAGUGGAAAAGGGGAAAUCCAAGAAGCUGAAGUCG | |
| | | GUGAAGGAGCUUUUGGGUAUCACCAUCAUGGAACGCUCCUCGUUCGA | |
| | | AAAGAACCCAAUCGAUUUCCUGGAAGCUAAGGGUUAUAAGGAAGUGA | |
| | | AAAAGGACCUGAUUAUCAAGCUGCCCAAGUACUCACUGUUCGAGCUG | |
| | | GAAAACGGUCGGAAAAGGAUGCUGGCCAGCGCCGGAGAACUCCAGAA | |
| | | GGGAAACGAACUGGCACUGCCGUCCAAAUACGUCAACUUCCUCUACC | |
| | | UUGCAUCCCAUUACGAAAAACUCAAGGGAUCGCCGGAGGACAACGAG | |
| | | CAGAAGCAGCUUUUCGUGGAGCAACACAAGCAUUACUUGGACGAGAU | |
| | | CAUCGAGCAGAUUUCCGAGUUCUCAAAGCGCGUGAUCCUGGCCGACG | |
| | | CAAAUCUGGACAAGGUCCUGUCCGCGUACAAUAAGCAUCGGGACAAG | |
| | | CCUAUCCGCGAACAGGCCGAGAACAUCAUCCAUCUGUUCACUCUGAC | |
| | | AAACCUGGGCGCACCCGCCGCGUUCAAGUACUUUGACACCACCAUCG | |
| | | AUAGGAAGCGAUACACCUCAACUAAGGAAGUGUUGGACGCGACCCUU | |
| | | AUCCAUCAGUCGAUCACCGGGCUGUACGAAACACGGAUCGACCUCAG | |
| | | CCAGUUGGGAGGCGACAAGCGCCCUGCGGCUACCAAGAAGGCCGGAC | |
| | | AGGCCAAGAAGAAGAAAUGA | |
| RNA-009 | amino | MAPKKKRKVGIHGVPAADKKYSIGLDIGTNSVGWAVITDEYKVPSKK | 5 |
| coding region | acid | FKVLGNTDRHSIKKNLIGALLFDSGETAEATRLKRTARRRYTRRKNR | |
| Nucleoplasmin | | ICYLQEIFSNEMAKVDDSFFHRLEESFLVEEDKKHERHPIFGNIVDE | |
| NLS | | VAYHEKYPTIYHLRKKLVDSTDKADLRLIYLALAHMIKFRGHFLIEG | |
| underlined | | DLNPDNSDVDKLFIQLVQTYNGLFEENPINASGVDAKAILSARLSKS | |
| SV40 NLS in | | RRLENLIAQLPGEKKNGLFGNLIALSLGLTPNFKSNFDLAEDAKLQL | |
| bold italics | | SKDTYDDDLDNLLAQIGDQYADLFLAAKNLSDAILLSDILRVNTEIT | |
| SpCas9 | | KAPLSASMTKRYDEHHGDLTLLKALVRGGLPEKYKEIFFDGSKNGYA | |
| polypeptide | | GYIDGGASQEEFYKFIKPILEKMDGTEELLVKLNREDLLRKGRTFDN | |
| in italics | | GSIPHQIHLGELHAILRRQEDFYPFLKDNREKIEKILTFRIPYYVGP | |
| | | LARGNSRFAWMTRKSEETITPWNFEEVVDKGASAQSFIERMTNFDKN | |
| | | LPNEKVLPKHSLLYEYFTVYNELTKVKYVTEGMRKPAFLSGEQKKAI | |
| | | VDLLFKTNRKVTVKQLKEDYFKKIECFDSVEISGVEDRFNASLGTYH | |
| | | DLLKIIKDKDFLDNEENEDILEDIVLTLTLFEDREMIEERLKTYAHL | |
| | | FDDKVMKGLKRRRYTGWGRLSRKLINGIRDKGSGKTILDFLKSDGFA | |
| | | NRNFMGLIHDDSLTFKEDIQKAQVSGQGDSLHEHIANTAGSPAIKKG | |
| | | ILQTVKVVDELVKVMGRHKPENIVIEMARENGTTGKGQKNSRERMKR | |
| | | IEEGIKELGSGILKEHPVENTQLGNEKLYLYYLGNGRDMYVDGELDI | |
| | | NRLSDYDVDHIVPQSFLKDDSIDNKVLTRSDKNRGKSDNVPSEEVVK | |
| | | KMKNYWRGLLNAKLITGRKFDNLTKAERGGLSELDKAGFIKRGLVET | |
| | | RQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDV | |
| | | RKMIAKSEQEIGKATAKYFFYSNIMNFFKTEITLANGEIRKRPLIET | |
| | | NGETGEIVWDKGRDFATVRKVLSMPGVNIVKKTEVGTGGFSKESILP | |
| | | KRNSDKLIARKKDWDPKKYGGFDSPTVAYSVLVVAKVEKGKSKKLKS | |
| | | VKELLGITIMERSSFEKNPIDFLEAKGYKEVKKDLIIKLPKYSLFEL | |
| | | ENGRKRMLASAGELQKGNELALPSKYVNFLYLASHYEKLKGSPEDNE | |
| | | QKQLFVEQHKHYLDEIIEQISEFSKRVILADANLDKVLSAYNKHRDK | |
| | | PIREQAENIIHLFTLTNLGAPAAFKYFDTTIDRKRYTSTKEVLDATL | |
| | | IHGSITGLYETRIDLSGLGGDKRPAATKKAGQAKKKK | |
| SpCas9 | Amino | DKKYSIGLDIGTNSVGWAVITDEYKVPSKKFKVLGNTDRHSIKKNLI | 6 |
| polypeptide | acid | GALLFDSGETAEATRLKRTARRRYTRRKNRICYLQEIFSNEMAKVDD | |
| | | SFFHRLEESFLVEEDKKHERHPIFGNIVDEVAYHEKYPTIYHLRKKL | |
| | | VDSTDKADLRLIYLALAHMIKFRGHFLIEGDLNPDNSDVDKLFIQLV | |
| | | QTYNQLFEENPINASGVDAKAILSARLSKSRRLENLIAQLPGEKKNG | |
| | | LFGNLIALSLGLTPNFKSNFDLAEDAKLQLSKDTYDDDLDNLLAQIG | |
| | | DQYADLFLAAKNLSDAILLSDILRVNTEITKAPLSASMIKRYDEHHQ | |
| | | DLTLLKALVRQQLPEKYKEIFFDQSKNGYAGYIDGGASQEEFYKFIK | |
| | | PILEKMDGTEELLVKLNREDLLRKQRTFDNGSIPHQIHLGELHAILR | |
| | | RQEDFYPFLKDNREKIEKILTFRIPYYVGPLARGNSRFAWMTRKSEE | |
| | | TITPWNFEEVVDKGASAQSFIERMTNFDKNLPNEKVLPKHSLLYEYF | |
| | | TVYNELTKVKYVTEGMRKPAFLSGEQKKAIVDLLFKTNRKVTVKQLK | |
| | | EDYFKKIECEDSVEISGVEDRFNASLGTYHDLLKIIKDKDFLDNEEN | |
| | | EDILEDIVLTLTLFEDREMIEERLKTYAHLFDDKVMKQLKRRRYTGW | |
| | | GRLSRKLINGIRDKQSGKTILDFLKSDGFANRNFMQLIHDDSLTFKE | |
| | | DIQKAQVSGQGDSLHEHIANLAGSPAIKKGILQTVKVVDELVKVMGR | |
| | | HKPENIVIEMARENQTTQKGQKNSRERMKRIEEGIKELGSQILKEHP | |
| | | VENTQLQNEKLYLYYLQNGRDMYVDQELDINRLSDYDVDHIVPQSFL | |
| | | KDDSIDNKVLTRSDKNRGKSDNVPSEEVVKKMKNYWRQLLNAKLITQ | |
| | | RKFDNLTKAERGGLSELDKAGFIKRQLVETRQITKHVAQILDSRMNT | |
| | | KYDENDKLTREVKVITLKSKLVSDFRKDFQFYKVREINNYHHAHDAY | |
| | | LNAVVGTALIKKYPKLESEFVYGDYKVYDVRKMIAKSEQEIGKATAK | |
| | | YFFYSNIMNFEKTEITLANGEIRKRPLIETNGETGEIVWDKGRDFAT | |
| | | VRKVLSMPQVNIVKKTEVQTGGFSKESILPKRNSDKLIARKKDWDPK | |
| | | KYGGFDSPTVAYSVLVVAKVEKGKSKKLKSVKELLGITIMERSSFEK | |
| | | NPIDFLEAKGYKEVKKDLIIKLPKYSLFELENGRKRMLASAGELQKG | |
| | | NELALPSKYVNFLYLASHYEKLKGSPEDNEQKQLFVEQHKHYLDEII | |
| | | EQISEFSKRVILADANLDKVLSAYNKHRDKPIREQAENIIHLFTLTN | |
| | | LGAPAAFKYFDTTIDRKRYTSTKEVLDATLIHQSITGLYETRIDLSQ | |
| | | LGGD | |
| nucleoplasmin | Amino | KRPAATKKAGQAKKKK | 7 |
| NLS | acid | | |
| SV40 NLS | Amino | PKKKRKV | 8 |
| | acid | | |
| RNA-009 | DNA | AGAGGAAATAAGAGAGAAAAGAAGAGTAAGAAGAAATATAAGAGCCA | 9 |
| 5′ UTR | | CC | |
| RNA-009 | RNA | AGAGGAAAUAAGAGAGAAAAGAAGAGUAAGAAGAAAUAUAAGAGCCA | 10 |
| 5′ UTR | | CC | |
| RNA-009 | DNA | GCGGCCGCTTAATTAAGCTGCCTTCTGCGGGGCTTGCCTTCTGGCCA | 11 |
| 3′ UTR | | TGCCCTTCTTCTCTCCCTTGCACCTGTACCTCTTGGTCTTTGAATAA | |
| | | AGCCTGAGTAGGAAGTCTAG | |
| RNA-009 | RNA | GCGGCCGCUUAAUUAAGCUGCCUUCUGCGGGGCUUGCCUUCUGGCCA | 12 |
| 3′ UTR | | UGCCCUUCUUCUCUCCCUUGCACCUGUACCUCUUGGUCUUUGAAUAA | |
| | | AGCCUGAGUAGGAAGUCUAG | |
| Poly-A tail | RNA | AAAAAAAAAAAAAAAAAAAAAAAAAAAAAAAAAAAAAAAAAAAAAAA | 13 |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAAAAAAA | |
| RNA-009.2 | RNA | AGGAAAUAAGAGAGAAAAGAAGAGUAAGAAGAAAUAUAAGAGCCACC | 14 |
| Full-length | | AUGGCCCCUAAGAAGAAGAGAAAAGUCGGAAUUCACGGAGUCCCCGC | |
| mRNA | | CGCCGACAAAAAGUACUCCAUUGGCCUUGAUAUUGGAACCAACUCCG | |
| 5′ UTR IN | | UGGGUUGGGCCGUGAUCACUGACGAGUACAAGGUGCCGUCCAAGAAG | |
| BOLD | | UUCAAGGUGCUGGGGAACACUGACCGGCACUCAAUUAAGAAGAACCU | |
| 3′ UTR IN | | GAUUGGGGCGCUGCUGUUCGACUCCGGAGAAACCGCGGAGGCUACCC | |
| BOLD ITALICS | | GCCUGAAGCGGACUGCCCGGCGGAGAUACACGCGCAGGAAGAACCGG | |
| POLY-A TAIL | | AUUUGCUACCUCCAAGAAAUCUUCAGCAACGAAAUGGCAAAGGUGGA | |
| IN UNDERLINE | | CGAUUCCUUCUUCCAUCGCCUGGAAGAGAGCUUCCUGGUGGAAGAGG | |
| | | ACAAGAAGCACGAAAGACACCCGAUUUUCGGCAACAUCGUGGAUGAG | |
| | | GUCGCAUACCACGAAAAGUACCCCACCAUCUAUCAUCUUCGGAAGAA | |
| | | GCUGGUCGACUCCACCGAUAAGGCCGAUCUGCGCCUGAUCUACUUGG | |
| | | CGCUGGCUCACAUGAUUAAGUUCAGAGGACACUUUCUGAUAGAGGGC | |
| | | GACCUCAAUCCCGAUAACUCCGACGUGGAUAAGCUGUUCAUCCAACU | |
| | | GGUGCAGACGUACAACCAACUGUUUGAAGAGAAUCCAAUCAACGCCA | |
| | | GCGGGGUGGACGCCAAGGCCAUCCUGUCCGCCCGGCUGUCAAAGUCC | |
| | | AGACGCCUGGAGAAUCUCAUCGCGCAACUCCCUGGCGAAAAAAAGAA | |
| | | CGGACUCUUCGGGAAUCUGAUUGCUCUGUCCCUGGGGCUCACUCCGA | |
| | | ACUUCAAGUCGAACUUCGACCUGGCGGAGGACGCUAAGCUGCAGCUG | |
| | | UCCAAGGACACCUACGAUGACGAUCUGGAUAACCUUCUGGCCCAGAU | |
| | | CGGGGAUCAAUACGCCGAUCUCUUCCUGGCCGCAAAGAACUUGUCGG | |
| | | AUGCUAUUCUGCUGAGCGACAUUCUGCGGGUCAAUACUGAAAUCACC | |
| | | AAGGCGCCCCUGUCGGCCAGCAUGAUCAAGCGCUACGACGAACACCA | |
| | | CCAAGACCUGACUCUGCUGAAGGCCCUCGUGCGCCAGCAGCUGCCUG | |
| | | AAAAGUACAAGGAGAUUUUCUUCGACCAGUCCAAGAACGGAUACGCC | |
| | | GGAUACAUUGACGGAGGGGCCAGCCAGGAGGAAUUUUACAAAUUCAU | |
| | | CAAGCCCAUUCUCGAGAAAAUGGACGGAACCGAAGAGUUGCUCGUGA | |
| | | AGCUGAACAGAGAGGAUCUCCUCCGGAAGCAGCGGACCUUCGACAAC | |
| | | GGUUCCAUCCCGCACCAAAUCCACCUGGGCGAAUUGCACGCCAUCCU | |
| | | CCGGCGGCAGGAAGAUUUCUACCCAUUCUUGAAGGACAAUCGCGAAA | |
| | | AGAUCGAAAAGAUCUUGACUUUCCGCAUCCCGUACUACGUGGGCCCU | |
| | | CUGGCCCGCGGCAACUCCCGCUUCGCUUGGAUGACACGGAAGUCCGA | |
| | | GGAAACCAUUACGCCCUGGAACUUCGAGGAAGUGGUGGACAAGGGGG | |
| | | CGUCCGCCCAGAGCUUCAUCGAACGCAUGACCAAUUUCGACAAGAAC | |
| | | CUCCCGAACGAAAAAGUGCUGCCAAAGCACUCGCUCCUCUACGAAUA | |
| | | CUUCACCGUGUACAACGAGCUGACUAAGGUCAAAUACGUGACUGAGG | |
| | | GAAUGCGGAAGCCGGCCUUCCUGUCGGGAGAGCAGAAGAAGGCCAUA | |
| | | GUGGACUUGCUUUUCAAGACUAACCGGAAGGUCACUGUGAAGCAACU | |
| | | CAAGGAGGACUACUUCAAGAAGAUCGAGUGUUUCGACUCGGUGGAGA | |
| | | UCUCGGGUGUCGAGGACCGCUUCAACGCCUCCCUGGGAACUUACCAC | |
| | | GAUCUGCUGAAGAUCAUCAAGGACAAGGACUUCCUCGAUAACGAAGA | |
| | | AAAUGAGGACAUCCUCGAGGAUAUCGUGCUGACCCUGACCUUGUUCG | |
| | | AGGAUAGGGAGAUGAUCGAGGAGCGGCUCAAGACCUACGCCCACCUG | |
| | | UUUGACGACAAAGUGAUGAAGCAACUGAAACGGCGGAGGUAUACCGG | |
| | | CUGGGGUCGGCUGUCCCGCAAGCUGAUCAACGGGAUCAGGGACAAGC | |
| | | AGUCCGGAAAGACCAUCCUCGACUUCCUUAAGUCCGACGGAUUCGCG | |
| | | AACCGCAACUUCAUGCAACUUAUCCACGACGACUCGCUGACAUUCAA | |
| | | GGAAGAUAUCCAGAAGGCCCAGGUGUCCGGACAGGGGGACUCGCUUC | |
| | | AUGAGCACAUCGCUAACCUGGCCGGAUCCCCCGCCAUAAAAAAGGGC | |
| | | AUUCUGCAGACCGUCAAAGUGGUGGAUGAGCUGGUCAAGGUCAUGGG | |
| | | CCGGCAUAAGCCGGAAAACAUCGUCAUCGAGAUGGCCCGCGAGAACC | |
| | | AGACUACGCAGAAGGGCCAGAAGAACUCCCGGGAGCGGAUGAAGCGG | |
| | | AUUGAAGAGGGCAUCAAGGAGCUCGGCAGCCAGAUUCUGAAGGAACA | |
| | | UCCCGUGGAAAACACCCAGCUGCAAAACGAAAAGCUCUAUUUGUACU | |
| | | AUCUGCAAAACGGACGCGAUAUGUACGUGGAUCAGGAGCUGGACAUU | |
| | | AACAGACUGAGCGACUAUGACGUGGAUCACAUUGUGCCUCAAAGCUU | |
| | | CCUCAAGGACGACUCAAUUGACAACAAGGUCCUGACCAGAAGCGACA | |
| | | AGAACAGAGGAAAGUCGGAUAAUGUGCCGUCCGAAGAAGUGGUCAAG | |
| | | AAGAUGAAGAAUUACUGGAGACAGCUCCUGAAUGCGAAGCUCAUUAC | |
| | | CCAGCGGAAGUUCGAUAACCUGACCAAGGCCGAAAGGGGUGGACUGU | |
| | | CCGAACUCGACAAAGCUGGCUUCAUCAAGCGCCAACUGGUCGAAACC | |
| | | AGGCAGAUCACCAAGCACGUCGCCCAGAUUCUGGACAGCCGCAUGAA | |
| | | CACUAAGUACGACGAGAACGAUAAGCUGAUCCGCGAAGUGAAGGUCA | |
| | | UCACCCUGAAGUCCAAGCUCGUGUCCGACUUUCGGAAGGAUUUCCAG | |
| | | UUUUACAAGGUCCGCGAGAUCAACAACUACCAUCACGCCCACGACGC | |
| | | GUACCUUAACGCAGUCGUGGGAACGGCUCUUAUCAAGAAGUACCCAA | |
| | | AGCUGGAGUCGGAAUUUGUGUACGGAGACUACAAAGUGUACGACGUG | |
| | | CGCAAGAUGAUCGCCAAAUCUGAGCAAGAGAUCGGGAAGGCAACCGC | |
| | | CAAAUACUUCUUCUACUCAAACAUUAUGAAUUUUUUCAAAACUGAGA | |
| | | UUACCCUGGCUAACGGAGAAAUUCGGAAGCGCCCCCUGAUUGAAACC | |
| | | AACGGAGAAACUGGAGAAAUUGUGUGGGACAAGGGACGGGACUUCGC | |
| | | CACCGUCCGCAAGGUCCUCUCAAUGCCCCAAGUCAACAUCGUGAAAA | |
| | | AGACCGAAGUGCAAACCGGCGGCUUCUCAAAGGAGUCCAUCCUGCCU | |
| | | AAGCGCAACAGCGACAAGCUGAUUGCCAGGAAGAAGGACUGGGACCC | |
| | | GAAGAAGUACGGAGGAUUUGAUUCCCCUACCGUGGCCUACUCCGUGC | |
| | | UCGUGGUGGCCAAAGUGGAAAAGGGGAAAUCCAAGAAGCUGAAGUCG | |
| | | GUGAAGGAGCUUUUGGGUAUCACCAUCAUGGAACGCUCCUCGUUCGA | |
| | | AAAGAACCCAAUCGAUUUCCUGGAAGCUAAGGGUUAUAAGGAAGUGA | |
| | | AAAAGGACCUGAUUAUCAAGCUGCCCAAGUACUCACUGUUCGAGCUG | |
| | | GAAAACGGUCGGAAAAGGAUGCUGGCCAGCGCCGGAGAACUCCAGAA | |
| | | GGGAAACGAACUGGCACUGCCGUCCAAAUACGUCAACUUCCUCUACC | |
| | | UUGCAUCCCAUUACGAAAAACUCAAGGGAUCGCCGGAGGACAACGAG | |
| | | CAGAAGCAGCUUUUCGUGGAGCAACACAAGCAUUACUUGGACGAGAU | |
| | | CAUCGAGCAGAUUUCCGAGUUCUCAAAGCGCGUGAUCCUGGCCGACG | |
| | | CAAAUCUGGACAAGGUCCUGUCCGCGUACAAUAAGCAUCGGGACAAG | |
| | | CCUAUCCGCGAACAGGCCGAGAACAUCAUCCAUCUGUUCACUCUGAC | |
| | | AAACCUGGGCGCACCCGCCGCGUUCAAGUACUUUGACACCACCAUCG | |
| | | AUAGGAAGCGAUACACCUCAACUAAGGAAGUGUUGGACGCGACCCUU | |
| | | AUCCAUCAGUCGAUCACCGGGCUGUACGAAACACGGAUCGACCUCAG | |
| | | CCAGUUGGGAGGCGACAAGCGCCCUGCGGCUACCAAGAAGGCCGGAC | |
| | | AGGCCAAGAAGAAGAAAUGAGCGGCCGCUUAAUUAAGCUGCCUUCUG | |
| | | CGGGGCUUGCCUUCUGGCCAUGCCCUUCUUCUCUCCCUUGCACCUGU | |
| | | ACCUCUUGGUCUUUGAAUAAAGCCUGAGUAGGAAGUCUAGAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAAAAAAAAAAAAAAA | |
| RNA-009.2 | RNA | AGGAAAUAAGAGAGAAAAGAAGAGUAAGAAGAAAUAUAAGAGCCACC | 15 |
| 5′ UTR | | | |
| Parent mRNA | DNA | AGGAAATAAGAGAGAAAAGAAGAGTAAGAAGAAATATAAGAGCCACC | 16 |
| | | ATGGCCCCAAAGAAGAAGCGGAAGGTCGGTATCCACGGAGTCCCAGC | |
| | | AGCCGACAAGAAGTACAGCATCGGCCTGGACATCGGCACCAACTCTG | |
| | | TGGGCTGGGCCGTGATCACCGACGAGTACAAGGTGCCCAGCAAGAAA | |
| | | TTCAAGGTGCTGGGCAACACCGACCGGCACAGCATCAAGAAGAACCT | |
| | | GATCGGAGCCCTGCTGTTCGACAGCGGCGAAACAGCCGAGGCCACCC | |
| | | GGCTGAAGAGAACCGCCAGAAGAAGATACACCAGACGGAAGAACCGG | |
| | | ATCTGCTATCTGCAAGAGATCTTCAGCAACGAGATGGCCAAGGTGGA | |
| | | CGACAGCTTCTTCCACAGACTGGAAGAGTCCTTCCTGGTGGAAGAGG | |
| | | ACAAGAAGCACGAGAGACACCCCATCTTCGGCAACATCGTGGACGAG | |
| | | GTGGCCTACCACGAGAAGTACCCCACCATCTACCACCTGAGAAAGAA | |
| | | ACTGGTGGACAGCACCGACAAGGCCGACCTGAGACTGATCTACCTGG | |
| | | CCCTGGCCCACATGATCAAGTTCAGAGGCCACTTCCTGATCGAGGGC | |
| | | GACCTGAACCCCGACAACAGCGACGTGGACAAGCTGTTCATCCAGCT | |
| | | GGTGCAGACCTACAACCAGCTGTTCGAGGAAAACCCCATCAACGCCA | |
| | | GCGGCGTGGACGCCAAGGCTATCCTGTCTGCCAGACTGAGCAAGAGC | |
| | | AGAAGGCTGGAAAATCTGATCGCCCAGCTGCCCGGCGAGAAGAAGAA | |
| | | CGGCCTGTTCGGCAACCTGATTGCCCTGAGCCTGGGCCTGACCCCCA | |
| | | ACTTCAAGAGCAACTTCGACCTGGCCGAGGATGCCAAACTGCAGCTG | |
| | | AGCAAGGACACCTACGACGACGACCTGGACAACCTGCTGGCCCAGAT | |
| | | CGGCGACCAGTACGCCGACCTGTTCCTGGCCGCCAAGAACCTGTCTG | |
| | | ACGCCATCCTGCTGAGCGACATCCTGAGAGTGAACACCGAGATCACC | |
| | | AAGGCCCCCCTGAGCGCCTCTATGATCAAGAGATACGACGAGCACCA | |
| | | CCAGGACCTGACCCTGCTGAAAGCTCTCGTGCGGCAGCAGCTGCCTG | |
| | | AGAAGTACAAAGAAATCTTCTTCGACCAGAGCAAGAACGGCTACGCC | |
| | | GGCTACATCGATGGCGGCGCTAGCCAGGAAGAGTTCTACAAGTTCAT | |
| | | CAAGCCCATCCTGGAAAAGATGGACGGCACCGAGGAACTGCTCGTGA | |
| | | AGCTGAACAGAGAGGACCTGCTGAGAAAGCAGAGAACCTTCGACAAC | |
| | | GGCAGCATCCCCCACCAGATCCACCTGGGAGAGCTGCACGCTATCCT | |
| | | GAGAAGGCAGGAAGATTTTTACCCATTCCTGAAGGACAACCGGGAAA | |
| | | AGATCGAGAAGATCCTGACCTTCAGGATCCCCTACTACGTGGGCCCC | |
| | | CTGGCCAGAGGCAACAGCAGATTCGCCTGGATGACCAGAAAGAGCGA | |
| | | GGAAACCATCACCCCCTGGAACTTCGAGGAAGTGGTGGACAAGGGCG | |
| | | CCAGCGCCCAGAGCTTCATCGAGAGAATGACAAACTTCGATAAGAAC | |
| | | CTGCCCAACGAGAAGGTGCTGCCCAAGCACAGCCTGCTGTACGAGTA | |
| | | CTTCACCGTGTACAACGAGCTGACCAAAGTGAAATACGTGACCGAGG | |
| | | GAATGAGAAAGCCCGCCTTCCTGAGCGGCGAGCAGAAAAAGGCCATC | |
| | | GTGGACCTGCTGTTCAAGACCAACAGAAAAGTGACCGTGAAGCAGCT | |
| | | GAAAGAGGACTACTTCAAGAAAATCGAGTGCTTCGACTCCGTGGAAA | |
| | | TCTCCGGCGTGGAAGATAGATTCAACGCCTCCCTGGGCACATACCAC | |
| | | GATCTGCTGAAAATTATCAAGGACAAGGACTTCCTGGATAACGAAGA | |
| | | GAACGAGGACATTCTGGAAGATATCGTGCTGACCCTGACACTGTTTG | |
| | | AGGACCGCGAGATGATCGAGGAAAGGCTGAAAACCTACGCTCACCTG | |
| | | TTCGACGACAAAGTGATGAAGCAGCTGAAGAGAAGGCGGTACACCGG | |
| | | CTGGGGCAGGCTGAGCAGAAAGCTGATCAACGGCATCAGAGACAAGC | |
| | | AGAGCGGCAAGACAATCCTGGATTTCCTGAAGTCCGACGGCTTCGCC | |
| | | AACCGGAACTTCATGCAGCTGATCCACGACGACAGCCTGACATTCAA | |
| | | AGAGGACATCCAGAAAGCCCAGGTGTCCGGCCAGGGCGACTCTCTGC | |
| | | ACGAGCATATCGCTAACCTGGCCGGCAGCCCCGCTATCAAGAAGGGC | |
| | | ATCCTGCAGACAGTGAAGGTGGTGGACGAGCTCGTGAAAGTGATGGG | |
| | | CAGACACAAGCCCGAGAACATCGTGATCGAGATGGCTAGAGAGAACC | |
| | | AGACCACCCAGAAGGGACAGAAGAACTCCCGCGAGAGGATGAAGAGA | |
| | | ATCGAAGAGGGCATCAAAGAGCTGGGCAGCCAGATCCTGAAAGAACA | |
| | | CCCCGTGGAAAACACCCAGCTGCAGAACGAGAAGCTGTACCTGTACT | |
| | | ACCTGCAGAATGGCCGGGATATGTACGTGGACCAGGAACTGGACATC | |
| | | AACAGACTGTCCGACTACGATGTGGACCATATCGTGCCTCAGAGCTT | |
| | | TCTGAAGGACGACTCCATCGATAACAAAGTGCTGACTCGGAGCGACA | |
| | | AGAACAGAGGCAAGAGCGACAACGTGCCCTCCGAAGAGGTCGTGAAG | |
| | | AAGATGAAGAACTACTGGCGACAGCTGCTGAACGCCAAGCTGATTAC | |
| | | CCAGAGGAAGTTCGATAACCTGACCAAGGCCGAGAGAGGCGGCCTGA | |
| | | GCGAGCTGGATAAGGCCGGCTTCATCAAGAGGCAGCTGGTGGAAACC | |
| | | AGACAGATCACAAAGCACGTGGCACAGATCCTGGACTCCCGGATGAA | |
| | | CACTAAGTACGACGAAAACGATAAGCTGATCCGGGAAGTGAAAGTGA | |
| | | TCACCCTGAAGTCCAAGCTGGTGTCCGATTTCCGGAAGGATTTCCAG | |
| | | TTTTACAAAGTGCGCGAGATCAACAACTACCACCACGCCCACGACGC | |
| | | CTACCTGAACGCCGTCGTGGGAACCGCCCTGATCAAAAAGTACCCTA | |
| | | AGCTGGAAAGCGAGTTCGTGTACGGCGACTACAAGGTGTACGACGTG | |
| | | CGGAAGATGATCGCCAAGAGCGAGCAGGAAATCGGCAAGGCTACCGC | |
| | | CAAGTACTTCTTCTACAGCAACATCATGAACTTTTTCAAGACCGAAA | |
| | | TCACCCTGGCCAACGGCGAGATCAGAAAGCGCCCTCTGATCGAGACA | |
| | | AACGGCGAAACCGGGGAGATCGTGTGGGATAAGGGCAGAGACTTCGC | |
| | | CACAGTGCGAAAGGTGCTGAGCATGCCCCAAGTGAATATCGTGAAAA | |
| | | AGACCGAGGTGCAGACAGGCGGCTTCAGCAAAGAGTCTATCCTGCCC | |
| | | AAGAGGAACAGCGACAAGCTGATCGCCAGAAAGAAGGACTGGGACCC | |
| | | CAAGAAGTACGGCGGCTTCGACAGCCCTACCGTGGCCTACTCTGTGC | |
| | | TGGTGGTGGCTAAGGTGGAAAAGGGCAAGTCCAAGAAACTGAAGAGT | |
| | | GTGAAAGAGCTGCTGGGGATCACCATCATGGAAAGAAGCAGCTTTGA | |
| | | GAAGAACCCTATCGACTTTCTGGAAGCCAAGGGCTACAAAGAAGTGA | |
| | | AAAAGGACCTGATCATCAAGCTGCCTAAGTACTCCCTGTTCGAGCTG | |
| | | GAAAACGGCAGAAAGAGAATGCTGGCCTCTGCCGGCGAACTGCAGAA | |
| | | GGGAAACGAGCTGGCCCTGCCTAGCAAATATGTGAACTTCCTGTACC | |
| | | TGGCCTCCCACTATGAGAAGCTGAAGGGCAGCCCTGAGGACAACGAA | |
| | | CAGAAACAGCTGTTTGTGGAACAGCATAAGCACTACCTGGACGAGAT | |
| | | CATCGAGCAGATCAGCGAGTTCTCCAAGAGAGTGATCCTGGCCGACG | |
| | | CCAATCTGGACAAGGTGCTGTCTGCCTACAACAAGCACAGGGACAAG | |
| | | CCTATCAGAGAGCAGGCCGAGAATATCATCCACCTGTTCACCCTGAC | |
| | | AAACCTGGGCGCTCCTGCCGCCTTCAAGTACTTTGACACCACCATCG | |
| | | ACCGGAAGAGGTACACCAGCACCAAAGAGGTGCTGGACGCCACCCTG | |
| | | ATCCACCAGAGCATCACCGGCCTGTACGAGACAAGAATCGACCTGTC | |
| | | TCAGCTGGGAGGCGACAAGAGACCTGCCGCCACTAAGAAGGCCGGAC | |
| | | AGGCCAAAAAGAAGAAGTGAGCGGCCGCTTAATTAAGCTGCCTTCTG | |
| | | CGGGGCTTGCCTTCTGGCCATGCCCTTCTTCTCTCCCTTGCACCTGT | |
| | | ACCTCTTGGTCTTTGAATAAAGCCTGAGTAGGAAGTCTAGAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAA | |
| Parent mRNA | RNA | AGGAAAUAAGAGAGAAAAGAAGAGUAAGAAGAAAUAUAAGAGCCACC | 17 |
| Full-length | | AUGGCCCCAAAGAAGAAGCGGAAGGUCGGUAUCCACGGAGUCCCAGC | |
| mRNA | | AGCCGACAAGAAGUACAGCAUCGGCCUGGACAUCGGCACCAACUCUG | |
| 5′ UTR IN | | UGGGCUGGGCCGUGAUCACCGACGAGUACAAGGUGCCCAGCAAGAAA | |
| BOLD | | UUCAAGGUGCUGGGCAACACCGACCGGCACAGCAUCAAGAAGAACCU | |
| 3′ UTR IN | | GAUCGGAGCCCUGCUGUUCGACAGCGGCGAAACAGCCGAGGCCACCC | |
| BOLD ITALICS | | GGCUGAAGAGAACCGCCAGAAGAAGAUACACCAGACGGAAGAACCGG | |
| POLY-A TAIL | | AUCUGCUAUCUGCAAGAGAUCUUCAGCAACGAGAUGGCCAAGGUGGA | |
| IN UNDERLINE | | CGACAGCUUCUUCCACAGACUGGAAGAGUCCUUCCUGGUGGAAGAGG | |
| | | ACAAGAAGCACGAGAGACACCCCAUCUUCGGCAACAUCGUGGACGAG | |
| | | GUGGCCUACCACGAGAAGUACCCCACCAUCUACCACCUGAGAAAGAA | |
| | | ACUGGUGGACAGCACCGACAAGGCCGACCUGAGACUGAUCUACCUGG | |
| | | CCCUGGCCCACAUGAUCAAGUUCAGAGGCCACUUCCUGAUCGAGGGC | |
| | | GACCUGAACCCCGACAACAGCGACGUGGACAAGCUGUUCAUCCAGCU | |
| | | GGUGCAGACCUACAACCAGCUGUUCGAGGAAAACCCCAUCAACGCCA | |
| | | GCGGCGUGGACGCCAAGGCUAUCCUGUCUGCCAGACUGAGCAAGAGC | |
| | | AGAAGGCUGGAAAAUCUGAUCGCCCAGCUGCCCGGCGAGAAGAAGAA | |
| | | CGGCCUGUUCGGCAACCUGAUUGCCCUGAGCCUGGGCCUGACCCCCA | |
| | | ACUUCAAGAGCAACUUCGACCUGGCCGAGGAUGCCAAACUGCAGCUG | |
| | | AGCAAGGACACCUACGACGACGACCUGGACAACCUGCUGGCCCAGAU | |
| | | CGGCGACCAGUACGCCGACCUGUUCCUGGCCGCCAAGAACCUGUCUG | |
| | | ACGCCAUCCUGCUGAGCGACAUCCUGAGAGUGAACACCGAGAUCACC | |
| | | AAGGCCCCCCUGAGCGCCUCUAUGAUCAAGAGAUACGACGAGCACCA | |
| | | CCAGGACCUGACCCUGCUGAAAGCUCUCGUGCGGCAGCAGCUGCCUG | |
| | | AGAAGUACAAAGAAAUCUUCUUCGACCAGAGCAAGAACGGCUACGCC | |
| | | GGCUACAUCGAUGGCGGCGCUAGCCAGGAAGAGUUCUACAAGUUCAU | |
| | | CAAGCCCAUCCUGGAAAAGAUGGACGGCACCGAGGAACUGCUCGUGA | |
| | | AGCUGAACAGAGAGGACCUGCUGAGAAAGCAGAGAACCUUCGACAAC | |
| | | GGCAGCAUCCCCCACCAGAUCCACCUGGGAGAGCUGCACGCUAUCCU | |
| | | GAGAAGGCAGGAAGAUUUUUACCCAUUCCUGAAGGACAACCGGGAAA | |
| | | AGAUCGAGAAGAUCCUGACCUUCAGGAUCCCCUACUACGUGGGCCCC | |
| | | CUGGCCAGAGGCAACAGCAGAUUCGCCUGGAUGACCAGAAAGAGCGA | |
| | | GGAAACCAUCACCCCCUGGAACUUCGAGGAAGUGGUGGACAAGGGCG | |
| | | CCAGCGCCCAGAGCUUCAUCGAGAGAAUGACAAACUUCGAUAAGAAC | |
| | | CUGCCCAACGAGAAGGUGCUGCCCAAGCACAGCCUGCUGUACGAGUA | |
| | | CUUCACCGUGUACAACGAGCUGACCAAAGUGAAAUACGUGACCGAGG | |
| | | GAAUGAGAAAGCCCGCCUUCCUGAGCGGCGAGCAGAAAAAGGCCAUC | |
| | | GUGGACCUGCUGUUCAAGACCAACAGAAAAGUGACCGUGAAGCAGCU | |
| | | GAAAGAGGACUACUUCAAGAAAAUCGAGUGCUUCGACUCCGUGGAAA | |
| | | UCUCCGGCGUGGAAGAUAGAUUCAACGCCUCCCUGGGCACAUACCAC | |
| | | GAUCUGCUGAAAAUUAUCAAGGACAAGGACUUCCUGGAUAACGAAGA | |
| | | GAACGAGGACAUUCUGGAAGAUAUCGUGCUGACCCUGACACUGUUUG | |
| | | AGGACCGCGAGAUGAUCGAGGAAAGGCUGAAAACCUACGCUCACCUG | |
| | | UUCGACGACAAAGUGAUGAAGCAGCUGAAGAGAAGGCGGUACACCGG | |
| | | CUGGGGCAGGCUGAGCAGAAAGCUGAUCAACGGCAUCAGAGACAAGC | |
| | | AGAGCGGCAAGACAAUCCUGGAUUUCCUGAAGUCCGACGGCUUCGCC | |
| | | AACCGGAACUUCAUGCAGCUGAUCCACGACGACAGCCUGACAUUCAA | |
| | | AGAGGACAUCCAGAAAGCCCAGGUGUCCGGCCAGGGCGACUCUCUGC | |
| | | ACGAGCAUAUCGCUAACCUGGCCGGCAGCCCCGCUAUCAAGAAGGGC | |
| | | AUCCUGCAGACAGUGAAGGUGGUGGACGAGCUCGUGAAAGUGAUGGG | |
| | | CAGACACAAGCCCGAGAACAUCGUGAUCGAGAUGGCUAGAGAGAACC | |
| | | AGACCACCCAGAAGGGACAGAAGAACUCCCGCGAGAGGAUGAAGAGA | |
| | | AUCGAAGAGGGCAUCAAAGAGCUGGGCAGCCAGAUCCUGAAAGAACA | |
| | | CCCCGUGGAAAACACCCAGCUGCAGAACGAGAAGCUGUACCUGUACU | |
| | | ACCUGCAGAAUGGCCGGGAUAUGUACGUGGACCAGGAACUGGACAUC | |
| | | AACAGACUGUCCGACUACGAUGUGGACCAUAUCGUGCCUCAGAGCUU | |
| | | UCUGAAGGACGACUCCAUCGAUAACAAAGUGCUGACUCGGAGCGACA | |
| | | AGAACAGAGGCAAGAGCGACAACGUGCCCUCCGAAGAGGUCGUGAAG | |
| | | AAGAUGAAGAACUACUGGCGACAGCUGCUGAACGCCAAGCUGAUUAC | |
| | | CCAGAGGAAGUUCGAUAACCUGACCAAGGCCGAGAGAGGCGGCCUGA | |
| | | GCGAGCUGGAUAAGGCCGGCUUCAUCAAGAGGCAGCUGGUGGAAACC | |
| | | AGACAGAUCACAAAGCACGUGGCACAGAUCCUGGACUCCCGGAUGAA | |
| | | CACUAAGUACGACGAAAACGAUAAGCUGAUCCGGGAAGUGAAAGUGA | |
| | | UCACCCUGAAGUCCAAGCUGGUGUCCGAUUUCCGGAAGGAUUUCCAG | |
| | | UUUUACAAAGUGCGCGAGAUCAACAACUACCACCACGCCCACGACGC | |
| | | CUACCUGAACGCCGUCGUGGGAACCGCCCUGAUCAAAAAGUACCCUA | |
| | | AGCUGGAAAGCGAGUUCGUGUACGGCGACUACAAGGUGUACGACGUG | |
| | | CGGAAGAUGAUCGCCAAGAGCGAGCAGGAAAUCGGCAAGGCUACCGC | |
| | | CAAGUACUUCUUCUACAGCAACAUCAUGAACUUUUUCAAGACCGAAA | |
| | | UCACCCUGGCCAACGGCGAGAUCAGAAAGCGCCCUCUGAUCGAGACA | |
| | | AACGGCGAAACCGGGGAGAUCGUGUGGGAUAAGGGCAGAGACUUCGC | |
| | | CACAGUGCGAAAGGUGCUGAGCAUGCCCCAAGUGAAUAUCGUGAAAA | |
| | | AGACCGAGGUGCAGACAGGCGGCUUCAGCAAAGAGUCUAUCCUGCCC | |
| | | AAGAGGAACAGCGACAAGCUGAUCGCCAGAAAGAAGGACUGGGACCC | |
| | | CAAGAAGUACGGCGGCUUCGACAGCCCUACCGUGGCCUACUCUGUGC | |
| | | UGGUGGUGGCUAAGGUGGAAAAGGGCAAGUCCAAGAAACUGAAGAGU | |
| | | GUGAAAGAGCUGCUGGGGAUCACCAUCAUGGAAAGAAGCAGCUUUGA | |
| | | GAAGAACCCUAUCGACUUUCUGGAAGCCAAGGGCUACAAAGAAGUGA | |
| | | AAAAGGACCUGAUCAUCAAGCUGCCUAAGUACUCCCUGUUCGAGCUG | |
| | | GAAAACGGCAGAAAGAGAAUGCUGGCCUCUGCCGGCGAACUGCAGAA | |
| | | GGGAAACGAGCUGGCCCUGCCUAGCAAAUAUGUGAACUUCCUGUACC | |
| | | UGGCCUCCCACUAUGAGAAGCUGAAGGGCAGCCCUGAGGACAACGAA | |
| | | CAGAAACAGCUGUUUGUGGAACAGCAUAAGCACUACCUGGACGAGAU | |
| | | CAUCGAGCAGAUCAGCGAGUUCUCCAAGAGAGUGAUCCUGGCCGACG | |
| | | CCAAUCUGGACAAGGUGCUGUCUGCCUACAACAAGCACAGGGACAAG | |
| | | CCUAUCAGAGAGCAGGCCGAGAAUAUCAUCCACCUGUUCACCCUGAC | |
| | | AAACCUGGGCGCUCCUGCCGCCUUCAAGUACUUUGACACCACCAUCG | |
| | | ACCGGAAGAGGUACACCAGCACCAAAGAGGUGCUGGACGCCACCCUG | |
| | | AUCCACCAGAGCAUCACCGGCCUGUACGAGACAAGAAUCGACCUGUC | |
| | | UCAGCUGGGAGGCGACAAGAGACCUGCCGCCACUAAGAAGGCCGGAC | |
| | | AGGCCAAAAAGAAGAAGUGAGCGGCCGCUUAAUUAAGCUGCCUUCUG | |
| | | CGGGGCUUGCCUUCUGGCCAUGCCCUUCUUCUCUCCCUUGCACCUGU | |
| | | ACCUCUUGGUCUUUGAAUAAAGCCUGAGUAGGAAGUCUAGAAAAAAA | |
| | | AAAAAAAAAAAAAAAAAAAAAAAAAAAAAAAAAAAAAAAAAAAAAAA | |
| | | AAAAAAAAAAAAAAAAAAAAAAAAAA | |

What is claimed:

1. An mRNA comprising:
a 5′ untranslated region (UTR);
an open reading frame (ORF) comprising a nucleotide sequence that encodes a site-directed endonuclease, wherein the nucleotide sequence is at least 85% identical to a nucleotide sequence of SEQ ID NO: 4; and
a 3′ untranslated region (UTR).

2. The mRNA of claim 1, wherein the mRNA comprises at least one chemically modified nucleoside.

3. The mRNA of claim 2, wherein the chemically modified nucleoside is selected from pseudouridine, N1-methylpseudouridine, and 5-methoxyuridine.

4. The mRNA of claim 2 or 3, wherein the chemically modified nucleoside is N1-methylpseudouridine.

5. The mRNA of any one of claims 1-4, wherein at least about 80% of the uridines are chemically modified.

6. The mRNA of any one of claims 1-5, wherein 100% of the uridines are chemically modified.

7. The mRNA of claim 5 or 6, wherein the uridines are modified and/or replaced with N1-methylpseudouridine.

8. An mRNA comprising:
a 5′ UTR;
an ORF comprising a nucleotide sequence that encodes a site-directed endonuclease, wherein the nucleotide sequence is at least 85% identical to the nucleotide sequence of SEQ ID NO: 4; and
a 3′ UTR,
wherein 100% of the uridines of the mRNA are modified and/or replaced with N1-methylpseudouridine.

9. The mRNA of any one of claims 1-8, wherein the 5′ UTR comprises a nucleotide sequence of SEQ ID NO: 10 or SEQ ID NO: 15.

10. The mRNA of any one of claims 1-9, wherein the 3′ UTR comprises a nucleotide sequence of SEQ ID NO: 12.

11. The mRNA of any one of claims 1-10, wherein the mRNA further comprises a poly-A tail.

12. The mRNA of claim 11, wherein the poly-A tail is about 100 to about 1000, about 10 to about 500, about 10 to about 300, about 10 to about 200, about 50 to about 200, about 50 to about 150, about 100 to about 150, or about 120 to about 150 adenosine nucleotides.

13. The mRNA of any one of claims 1-12, wherein the mRNA comprises the nucleotide sequence of SEQ ID NO: 2 or SEQ ID NO: 14.

14. An mRNA comprising a nucleotide sequence of SEQ ID NO: 2 or SEQ ID NO: 14, wherein 100% of the uridines of the mRNA are modified and/or replaced with N1-methylpseudouridine.

15. The mRNA of any one of claims 1-14, wherein the mRNA comprises a 5′ cap.

16. The mRNA of any one of claims 1-15, wherein the 5′ cap is a cap-0, a cap-1, or a cap-2 structure.

17. A system for introducing a double-stranded DNA break (DSB) in a target gene in a cell, the system comprising:
(a) the mRNA of any one of claims 1-16; and
(b) at least one guide RNA (gRNA) directed to the target gene,
wherein the mRNA is translated when the mRNA contacts the cell and provides a site-directed endonuclease that combines with the gRNA to induce a DSB at a site in the target gene.

18. The system of claim 17, wherein the mRNA and the gRNA are individually formulated in a lipid nanoparticle (LNP).

19. The system of claim 17, wherein the mRNA and the gRNA are co-formulated in a lipid nanoparticle (LNP).

20. The system of any one of claims 17-19, wherein the LNP comprises one or more lipid moieties selected from: an amino lipid, an ionizable lipid, a neutral lipid, a PEG-lipid, a helper lipid, a cholesterol or derivative thereof.

21. A system for correcting a mutation in a target gene in a cell, the system comprising:
(a) the mRNA of any one of claims 1-16;
(b) at least one guide RNA (gRNA) directed to the target gene; and
(c) a donor polynucleotide,
wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

22. A pharmaceutical composition comprising:
the mRNA of any one of claims 1-16 or the system of any one of claims 17-20, and
a pharmaceutically acceptable carrier.

23. The pharmaceutical composition of claim 22, further comprising at least one gRNA directed to the target gene.

24. The pharmaceutical composition of claim 22 or 23, further comprising a donor polynucleotide.

25. A method for inducing a DSB in a target gene in a cell, the method comprising:
contacting the cell with:
(i) the mRNA of any one of claims 1-16 and at least one gRNA directed to the target gene;
(ii) the system of any one of claims 17-21; or
(iii) the pharmaceutical composition of claim 23 or 24,
wherein the mRNA is translated when the mRNA, the system, or the composition contacts the cell and provides a site-directed endonuclease that combines with the gRNA to induce a DSB at a site in the target gene.

26. A method of treating a patient with a disease by inducing a DSB in a target gene in a cell, the method comprising:
isolating a cell from the patient, and
contacting the cell with:
(i) the mRNA of any one of claims 1-16 and at least one gRNA directed to the target gene;
(ii) the system of any one of claims 17-21; or
(iii) the pharmaceutical composition of claim 23 or 24,
wherein the mRNA is translated when the mRNA, system, or composition contacts the cell and provides a site-directed endonuclease that combines with the gRNA to induce a DSB at a site in the target gene.

27. A method of treating a patient with a disease by inducing a DSB in a target gene in a cell, the method comprising:
administering to the patient an effective amount of:
(i) the mRNA of any one of claims 1-16 and at least one gRNA directed to the target gene;
(ii) the system of any one of claims 17-21; or
(iii) the pharmaceutical composition of claim 23 or 24,
wherein the mRNA is translated when the mRNA, system, or composition contacts the cell and provides a site-directed endonuclease that combines with the gRNA to induce a DSB at a site in the target gene.

28. A method for correcting a mutation in a target gene in a cell, the method comprising: contacting the cell with the mRNA of any one of claims 1-16, at least one gRNA directed to the target gene, and a donor polynucleotide, wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

29. A method of treating a patient with a disease by correcting a mutation in a target gene in a cell, the method comprising:
isolating a cell from the patient; and
contacting the cell with the mRNA of any one of claims 1-16, at least one gRNA directed to the target gene, and a donor polynucleotide,
wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

30. A method of treating a patient with a disease by correcting a mutation in a target gene in a cell, the method comprising:
administering to the patient an effective amount of the mRNA of any one of claims 1-16, at least one gRNA directed to the target gene, and a donor polynucleotide,
wherein the mRNA is translated when the mRNA contacts the cell and provides a site-directed endonuclease that combines with the gRNA to induce a DSB at a site in or near the mutation in the target gene, and wherein a non-homologous end-joining (NHEJ) DNA repair pathway inserts the donor polynucleotide into the DSB at a location proximal to the mutation, thereby correcting the mutation.

31. A kit for inducing a DSB in a target gene in a cell, the kit comprising:
a container comprising an mRNA of any one of claims 1-16, the system of any one of claims 17-21, or the pharmaceutical composition of claim 22-24, and
a package insert comprising instructions for use.
